SEQUENCE LISTING (58 sequences)

SEQ ID NO: 1 - protein, 507 aa, Microbacterium luteolum
MAQYDVAIIGAGSAGALIAARLSEDPARNVLLIEAGGRPSDPDILKPSMWPAIQHRSYDWDYKTTPQEGAAGRSFAWARGKGLGGSSLLHAMGYMRGHPADFAAWAEATGDERWSWEGLLPSFMANEDHVSGGDGIHGKDGPMPVWIPDDEVSPLTQAFMTAGNALGLPRIPDHNTGQMIGVTPNSLMIRDGRRVTVAEAWLTPEVCARPNLTIMTGTLTRRLKLEKSHVSAIELAGPEGLATVTASEIILSAGSLESPALLMRSGIGRENVLREAGVTCRVKAPELGLNLMDHLLGAGNLYATKKHLPPSRLQHSESMAYMRAGDFSAGGQPEIVVGCGVAPIVSESFTAPAPGNAYSFLFGVTHPTSRGEIRITGDAPDSPLIIDPRYLQTQNDRNLFRAALGAAREIGHRPELAEWRDHEILPKSLAASQDIDTFIAKAVITHHHPSGTCRMGKDEMSVVDADLRLRGLDNLYVVDGSVLPSLTAGPIHAAVQAIAENFTTGFK

SEQ ID NO: 2 - protein, 393 aa, Mesorhizobium loti
MMRYPEHADPVITLTAGPVNAYPEVLRGLGRTVLYDYDPAFQLLYEKVVDKAQKAMRLSNKPVILHGEPVLGLEAAAASLISPDDVVLNLASGVYGKGFGYWAKRYSPHLLEIEVPYNEAIDPQAVADMLKAHPEITVVSVCHHDTPSGTINPIDAIGALVSAHGAYLIVDAVSSFGGMKTHPEDCKADIYVTGPNKCLGAPPGLTMMGVSERAWAKMKANPLAPRASMLSIVDWENAWSRDKPFPFTPSVSEINGLDVALDLYLNEGPEAVWARHALTAKAMRAGVTAMGLSVWAASDSIASPTTTAVRTPDGVDEKALRQAARARYGVVFSSGRGETLGKLTRIGHMGPTAQPIYAIAALTALGGAMNAAGRKLAIGKGIEAALAVIDADA

SEQ ID NO: 3 - protein, 488 aa, Listeria seeligeri
MTDRRNLTTNQGVPIGDNQNSMTAGLKGPTLLEDYVLIEKLAHFDRERVPERVVHARGAGAHGKFVTKKSMKKYTKAQFLQEEGTETEVFARFSTVIHGQHSPETLRDPRGFSVKFYTEEGNYDFVGNNLPVFFIRDAIKFPDVIHSLKPDPRTNIQDGNRYWDFFSLTPEATTMITYLFSDEGTPASYREIRGSSVHAFKWINEEGKTVYVKLRWVPKAGIVNLSTDQAAQIQAKEFNHASRDLYEAIENGDYPEWDLYVQVLDPKDLDNYDFNPLDATKDWFEDVFPYEHVGTMTLNRNPDNIFAETESVGFNPGVLVPGMLPSEDRVLQGRLFSYSDTQRHRVGPNYLQLPINSPKTPVDNNQRDGQMPFKQQTSSINYEPNSYDTEPKENPAYIEPEQEIRGDISGRLVAEKPNNFGHAKEVWKRYSDAERAALVKNIVDDWEGVREDIKIRNLRNFYQVEPEFAERVAAGTGINLAEHVIDLK

SEQ ID NO: 4 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 5 - DNA, 1524 bp, Microbacterium luteolum
atggctcagtatgatgtggcgatcattggtgctggctctgccggcgccctgatagcagcgcgtctaagcgaagatccggcccgtaatgttctgctaatcgaagctggtgggagaccatcagacccggacattttaaagccctctatgtggcctgcgatccagcatcgcagctatgattgggattacaaaacgacacctcaggaaggtgccgctggcagaagttttgcctgggctcgcggcaaaggtcttggtggatcaagcctgctccacgctatgggctatatgcgtgggcacccggcagatttcgctgcatgggcagaagccacgggcgatgagcgctggagctgggaaggtttattgccatctttcatggcaaatgaagaccatgtgtcgggcggcgatggtattcacggcaaagacggtccgatgcccgtctggataccagacgacgaagtcagtccactgacacaggcattcatgacagcaggcaatgcactcggcctgccgcgtatcccggaccacaataccggccaaatgattggggtcacacccaattccctgatgatccgtgatgggcgtcgcgtcaccgttgctgaagcatggctcacgcccgaagtttgcgcgcgtccgaacctgacgatcatgacaggcacgttgacccgccgccttaaactggaaaaatcacacgtttcagcaattgaactcgcaggcccggaaggactagctaccgtcacggcaagcgaaatcattttgtccgctggttcattggaaagccccgcacttttaatgcgctctggcatagggcgcgaaaatgtactccgcgaagccggtgtcacttgccgggtcaaagcgccagaactggggcttaacctgatggatcatctgcttggggcgggtaatctttatgccactaaaaagcatttgcctccgtcccgtctgcaacattcggaaagcatggcgtatatgcgtgccggagatttctcagccggtggccagccggaaattgtcgttggctgcggcgtggctccaatcgtttccgagagctttactgcacctgctccgggtaacgcctattccttcctttttggtgttactcaccctacaagtcgaggcgaaatacgcataaccggagatgcaccggacagcccacttataattgaccctcgatatcttcagacccaaaatgatagaaacctgtttcgagcagcgctcggtgcggcacgtgaaatcggacatcgccctgaattagctgaatggcgtgaccacgaaatactccccaagtcgcttgcagcaagccaagacatcgacacttttattgcgaaggcagtgatcacacatcaccacccttccgggacatgccgtatgggtaaggatgaaatgtctgtcgttgatgctgatctgcgtctgcgcggattggacaacctgtatgtcgtcgatggttcagttttgccaagcctgactgcgggccctatccacgccgccgtacaggcaatcgcagaaaatttcacaacaggattcaaataa

SEQ ID NO: 6 - DNA, 1182 bp, Mesorhizobium loti
atgatgcgctatcccgaacatgccgatccggtcatcaccctgaccgcggggccggtgaacgcctatccagaagtgttgcgcggcctcggccgtacggtgctttatgattacgatcctgcattccagctcctttacgagaaggtggtcgacaaggcgcagaaggcgatgcggctgtcgaacaagccggtcatcctgcatggcgagccggtgctcggcctcgaagcggcggcggcgtcgctgatctcgcccgacgacgttgtgctcaatcttgcctcgggcgtctacggcaagggctttggctattgggcgaagcggtactcgccgcatctgctcgagatcgaagtgccctataacgaggcgatcgatccgcaggcggtcgccgacatgctcaaggcgcatccggagatcaccgttgtgtcggtctgtcatcacgacacgccgtcgggcaccatcaatccgatcgacgccatcggcgcgctggtctcggcgcatggcgcctatctgatcgtcgacgcggtctcgtcctttggcggcatgaagacgcatcccgaggattgcaaagccgatatttatgtcaccggccccaacaaatgcctgggcgcccctcccggcctcaccatgatgggcgtcagcgagcgggcctgggccaagatgaaggccaaccccctggcgccgcgcgcatcgatgctgagcattgtcgactgggaaaatgcctggtcgagggacaagccgtttccgttcacgccgtcggtctcggagatcaacgggctcgacgtcgcgctcgatctttacctcaatgagggaccggaggcggtatgggcgcgccatgcgctcaccgccaaggccatgcgagcgggcgtcaccgcgatgggcctgtcggtctgggccgccagcgacagcatcgcgtcgccgaccaccaccgccgtccgcacccccgatggtgtcgacgagaaggcgcttcgccaggccgcgcgtgcccgttacggcgtggtgttttcgtcggggcggggcgagacgttggggaagctgacgcgcatcggccatatggggccgaccgcccagccgatctacgcgatcgcggcgctgacggcacttggcggcgccatgaacgcggccggccggaaactcgcaatcggcaaaggcatcgaggcggcgctggccgtaatcgacgccgacgcctga

SEQ ID NO: 7 - DNA, 1467 bp, Listeria seeligeri
atgaccgatagaagaaacttaacgacgaatcaaggagtgccgattggggacaaccaaaattcgatgacagcgggattaaaaggaccaactttgttagaagattatgtgttaattgagaaattggcgcattttgatagagaacgtgttccagaacgtgtggtgcatgctcgtggtgctggtgcgcacggcaaatttgtaacgaaaaaaagcatgaaaaaatatacgaaagcacaatttttacaagaagaaggaacagagacagaggtttttgcgcgtttttctacagtaatccatggtcaacattcaccagaaacgcttcgtgatccacgtggtttttcggttaaattttatacggaagaaggtaactatgactttgtcggtaacaacttacctgtattctttatccgtgatgctatcaagtttcctgacgtaattcactccttgaagccagatccacgtactaacattcaagatggcaatcgttactgggacttctttagtttaactccggaagcgacgacgatgattacttatttatttagcgatgaggggactccggcatcttaccgcgaaattcgtggttcaagcgtacatgcctttaaatggataaacgaagaaggtaagactgtttatgtaaaactacgctgggttccaaaagcaggaatcgtcaatctttcaactgatcaagcagcacaaattcaagcaaaagaatttaaccatgctagtcgcgatttgtatgaagcaattgagaatggtgattatccagagtgggatttatatgtgcaagtgctagatccaaaagacttggataattacgacttcaatccgcttgatgcaaccaaagactggtttgaagatgtatttccatacgagcatgttggaacaatgacattaaatcgtaatccggataatatttttgctgaaacagaatcagttggctttaatccaggtgtgcttgtgccggggatgttaccttctgaggaccgtgtactacaggggcgattgttctcttactctgatacgcaaagacaccgcgttggacctaactacttacaattaccaatcaacagcccaaaaactcctgttgataacaaccaacgtgatggacagatgccgtttaaacagcaaacaagttcgattaattatgaaccaaatagttatgatacagaaccaaaagaaaaccctgcatatatcgagcctgagcaagaaattcgtggggatatctctggccgactagtggcagaaaagccaaataactttggtcatgctaaagaagtttggaagcgttactcagatgcagaacgtgcggctcttgtgaaaaatattgtagacgattgggaaggtgtgcgcgaagatattaagattcgcaacttgcgcaatttctatcaagtagagccggaatttgcagaacgtgtggctgctggaactggaattaaccttgctgaacatgtgatagatttaaaataa

SEQ ID NO: 8 - DNA, 1191 bp, Escherichia coli
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 9 - DNA, 1542 bp, Artificial
  Artificial Fragment for Pyridoxine Oxygenase
acaaaaaggataaaacaaatggctcaatatgatgtagctataataggagcagggagtgctggagcattaatagctgcgcgtctgagcgaagatccggcgcgtaacgtgctgctgattgaagcgggtggccgtccgagcgacccggatatcctgaagccgagcatgtggccggcgattcagcaccgtagctacgactgggattataagaccaccccgcaagagggtgcggcgggccgtagcttcgcgtgggcgcgtggtaaaggtctgggtggcagcagcctgctgcatgcgatgggctacatgcgtggtcacccggcggattttgcggcgtgggcggaggcgaccggtgacgaacgttggagctgggagggtctgctgccgagctttatggcgaacgaagaccacgtgagcggtggcgatggtattcacggcaaagacggtccgatgccggtgtggattccggacgatgaagttagcccgctgacccaggcgtttatgaccgcgggtaacgcgctgggtctgccgcgtatcccggatcacaacaccggccaaatgattggtgtgaccccgaacagcctgatgatccgtgacggccgtcgtgtgaccgttgcggaagcgtggctgaccccggaagtgtgcgcgcgtccgaacctgaccattatgaccggtaccctgacccgtcgtctgaagctggagaagagccacgttagcgcgattgaactggcgggtccggagggtctggcgaccgttaccgcgagcgaaatcattctgagcgcgggtagcctggagagcccggcgctgctgatgcgtagcggtattggccgtgaaaacgtgctgcgtgaagcgggcgtgacctgccgtgttaaggcgccggaactgggtctgaacctgatggatcacctgctgggtgcgggcaacctgtacgcgaccaagaaacacctgccgccgagccgtctgcagcacagcgagagcatggcgtatatgcgtgcgggtgacttcagcgcgggtggccaaccggaaattgtggttggttgcggcgtggcgccgatcgttagcgagagctttaccgcgccggcgccgggtaacgcgtacagcttcctgtttggtgttacccacccgaccagccgtggcgaaatccgtattaccggtgacgcgccggatagcccgctgatcattgatccgcgttatctgcagacccaaaacgaccgtaacctgttccgtgcggcgctgggtgcggcgcgtgaaattggtcaccgtccggagctggcggaatggcgtgatcacgagattctgccgaagagcctggcggcgagccaggacatcgatacctttattgcgaaagcggtgattacccatcatcacccgagcggtacctgccgtatgggtaaagacgagatgagcgtggttgacgcggatctgcgtctgcgtggcctggataacctgtatgtggttgacggtagcgttctgccgagcctgaccgcgggtccgattcacgcggcggttcaggcgattgcggaaaactttaccaccggcttcaagtaa

SEQ ID NO: 10 - DNA, 1199 bp, Artificial
  Artificial Fragment for Pyridoxamine-Pyruvate Transaminase
acaaaaaggataaaacaatgatgaggtatcccgaacacgctgatccagtaataactttaacagcaggacccgtaaacgcgtatccggaagttctgcgtggtctgggccgtaccgttctgtacgactatgatccggcgttccagctgctgtacgagaaggtggttgataaggcgcaaaaagcgatgcgtctgagcaacaaaccggtgattctgcacggcgaaccggttctgggtctggaagcggcggcggcgagcctgatcagcccggacgatgtggttctgaacctggcgagcggcgtgtacggcaagggctttggttactgggcgaaacgttatagcccgcacctgctggagattgaagtgccgtataacgaggcgattgacccgcaggcggttgcggatatgctgaaggcgcacccggaaatcaccgtggttagcgtgtgccaccacgacaccccgagcggtaccatcaacccgattgacgcgattggtgcgctggttagcgcgcacggtgcgtacctgattgtggacgcggttagcagcttcggtggcatgaagacccacccggaggactgcaaagcggatatctatgtgaccggtccgaacaaatgcctgggtgctccgccgggtctgaccatgatgggtgttagcgaacgtgcgtgggcgaagatgaaagcgaacccgctggcgccgcgtgcgagcatgctgagcattgtggactgggagaacgcgtggagccgtgataagccgttcccgtttaccccgagcgtgagcgaaatcaacggcctggacgttgcgctggatctgtatctgaacgaaggtccggaagcggtttgggcgcgtcatgcgctgaccgcgaaggcgatgcgtgcgggcgtgaccgcgatgggtctgagcgtttgggcggcgagcgatagcattgcgagcccgaccaccaccgcggtgcgtaccccggatggtgttgatgaaaaggcgctgcgtcaggcggcgcgtgcgcgttacggcgtggtttttagcagcggccgtggtgaaaccctgggtaaactgacccgtattggccacatgggtccgaccgcgcaaccgatttatgcgattgcggcgctgaccgcgctgggtggcgcgatgaacgcggcgggtcgtaagctggcgattggcaaaggtatcgaagcggcgctggcggttatcgacgcggatgcgtga

SEQ ID NO: 11 - DNA, 1485 bp, Artificial
  Artificial Fragment for Catalase
acaaaaaggataaaacaaatgacagataggagaaacctaacaactaaccaaggagttcccataggggataatcaaaacagcatgaccgcgggcctgaaaggcccgaccctgctggaagattacgtgctgatcgagaagctggcgcacttcgaccgtgaacgtgttccggagcgtgttgttcatgcgcgtggtgcgggtgcgcacggcaagttcgttaccaagaaaagcatgaagaaatataccaaagcgcagtttctgcaagaggaaggcaccgagaccgaagtgttcgcgcgttttagcaccgttattcacggtcaacacagcccggaaaccctgcgtgatccgcgtggcttcagcgttaaattttacaccgaggaaggtaactacgacttcgtgggcaacaacctgccggttttctttatccgtgatgcgattaagtttccggacgtgatccacagcctgaaaccggatccgcgtaccaacattcaggatggtaaccgttactgggacttctttagcctgaccccggaggcgaccaccatgatcacctacctgttcagcgacgaaggtaccccggcgagctatcgtgagatccgtggcagcagcgttcacgcgtttaagtggattaacgaggaaggtaaaaccgtgtacgttaaactgcgttgggtgccgaaggcgggcatcgttaacctgagcaccgatcaagcggcgcagattcaagcgaaagagttcaaccacgcgagccgtgacctgtatgaagcgattgagaacggtgattacccggagtgggacctgtatgtgcaggttctggatccgaaggacctggataactacgactttaacccgctggacgcgaccaaagattggttcgaagacgtgtttccgtatgagcacgttggcaccatgaccctgaaccgtaacccggacaacatcttcgcggagaccgaaagcgtgggttttaacccgggcgtgctggttccgggtatgctgccgagcgaagatcgtgtgctgcagggccgtctgttcagctacagcgacacccaacgtcaccgtgttggtccgaactatctgcagctgccgattaacagcccgaagaccccggtggataacaaccagcgtgacggccaaatgccgtttaaacagcaaaccagcagcatcaactacgaaccgaacagctatgataccgaaccgaaggagaacccggcgtacattgagccggaacaagagatccgtggtgacattagcggccgtctggtggcggagaaaccgaacaacttcggtcacgcgaaggaagtttggaaacgttacagcgatgcggagcgtgcggcgctggtgaagaacatcgttgacgattgggaaggcgtgcgtgaggacatcaaaattcgtaacctgcgtaacttctatcaggttgaaccggagtttgcggagcgtgttgcggcgggcaccggcatcaatctggcggagcatgttatcgacctgaaataa

SEQ ID NO: 12 - DNA, 29 bp, Artificial
  Primer Sequence
cgcggatccacaaaaaggataaaacaatg

SEQ ID NO: 13 - DNA, 31 bp, Artificial
  Primer Sequence
acgcgtcgacttacttgaagccggtggtaaa

SEQ ID NO: 14 - DNA, 29 bp, Artificial
  Primer Sequence
ccggaattcacaaaaaggataaaacaatg

SEQ ID NO: 15 - DNA, 28 bp, Artificial
  Primer Sequence
cgcggatcctcacgcatccgcgtcgata

SEQ ID NO: 16 - DNA, 45 bp, Artificial
  Primer Sequence
ggaattcacaaaaaggataaaacaatgtttgagaacattaccgcc

SEQ ID NO: 17 - DNA, 29 bp, Artificial
  Primer Sequence
cgcggatccttacagcactgccacaatcg

SEQ ID NO: 18 - protein, 392 aa, Mesorhizobium sp. YR577
MRYPDHTEPVITLTAGPVNAYPDVLRGLGRTVLYDYDPAFQLFYEKVIGKAQKAMRLSNKPVVLHGEPVMGLEAAAASLISRDDVVLNLASGVYGKGFGYWAKRYSPNLLEIEVPYNEAIDPQSVADMLKSHPEITVVSVCHHDTPSGTINPIDEIGAIVSAHGGYLIVDAVSSFGGMKTHPEDCKADIYVTGPNKCLGAPPGLTLMSVSDRAWTRMKANSAAPRASVLSILDWEHAWSKEKPFPFTPSVAEMNGLDVALDLYLNEGPEAVWARHALTAKATRAGAQAMGLSIWAANEKIASPTTTAIRTPDGVDEKALRQAARERYGVVFSSGRGETLGKLTRIGHMGPTAQPIYAIAALTALGGALNAAGRKLQVGKGIEAALAVIDADA

SEQ ID NO: 19 - protein, 392 aa, Pseudaminobacter salicylatoxidans
MRYKDNADPVITLTSGPVNAYADVLRGLSRTVLYDYDPAFQLFYENVVAKAQKAMRLSTRPVLLQGEPVMGLEAAAASLISSEDVVLNLASGVYGKGFGYWAKRYSPNLLEIETPYNESIDPQAVADMLKKHPEITVVSVCHHDTPSGTINPIDEIGAIVSAHGAYFIVDAVSSFGGMKTHPEDCKADLYVTGPNKCLGAPPGLTMMGVSDRAWARMKANPSAPRASVLSILDWEDAWSHEKPFPFTPSVAEINGLDVALDLYLNEGPEVVWARHALTAKATRAGALAMGLSLWAASEKIASPTTTSIRTPQGVDEAALRQAVRERYGVVFSSGRGETLGKLTRIGHMGPTAQPIYAVAALTALGGALNAMGQKLAVGRGIDAALAVIDADA

SEQ ID NO: 20 - protein, 392 aa, Bauldia litoralis
MRYALDADPVITLTSGPVNAYPDVLRALARTVLYDYDPAFQLHYEQVIGKMQTAMRTSNRPVILHGEPVLGLEAAAASLITADDTVLNLASGVYGKGFGYWAARYSPNQLEIEVPYNQAIDPQQVADMLKANPAISVVAVCHHDTPSGTINPIDEIGAIVADHGAYLIVDAVSSFGGMKTHPEDCKADIYVTGPNKCLGAPPALTMLGVSERAWQKMKDNPAAPRASMLSILDWENAWSRDEAFPFTPSVAEVNGLDVALDLYLAEGPEAVWARHALTAAATRAGILAMGLDLWAADAAIASPTTTAVRTPEGIDEAKLRATVRERYGVVMSSGRGETLGKLTRIGHMGPTAQPIYAIAALTAFGGALQALGHPAPVGKAIDAALAVIDAAA

SEQ ID NO: 21 - protein, 391 aa, Skermanella stibiiresistens
MTVERIVEPAFTLSSGPVDAYPAVLRALSRTVLYDFDPVFQHFYEAVTDKAKVALRTAAPPVILHGEPVLGLEAAAASLIARDDVVLNLASGVYGKGFGFWAKRYCAELVEIEVPYDDAIDPAAVEAAFRERPDIRVVSVCHHDTPSGTLNPIDEIGRIVAAHGAYLIVDAVSSFGGMDVHPEACQADIFVTGPNKCLGCPPGLTLLAVSGRAWDKMKANPDAPRASILSILDWENAWRRDQPFPFTPSISEVNALDAALDLYLEEGPEAVWRRHALTSKACRDGIRAMGMTIWPAREAIASPTTTAVRVPDGIDADALLATARDGFGVSFSSGRGETKGKLIRIGHMGRTAQPLYAVVGLAALGGALRRLGANVDAGAGMEAALAVISNQ

SEQ ID NO: 22 - protein, 394 aa, Rhizobium sp. AC44/96
MNQTKIADPIFTLTTGPVDAYPAVLRALSRPVLYDYDPAFLAFYEAVNAKVQRAFHTKTPPVILQGEPVLGLEAAAASLIAKKDVVLNLVSGVYGKGFGFWAARYARELVELEVPYNDVLTADAVADMLKKRPDIAVVALCHHDTPSGTVNPVAEIGAVVRAAGKLFIVDSVSAFGGMDVLPETACADIFVTGPNKCLGCPPGLSLLHVSEAAWEKIAANPDAPTASMLSISDWKRAHEAGQPFPFTPSVSEINALDAAMDLYLEEGEQAVWARHALTAKACRAGVQAAGLKLWAAREDIASPTCTAVAIPDGIDEAKLRASIRDRYGVVFSSGRAETLGKLTRIGHMGPTARPTYSLVSVAAIVGGLKAAGVKDLDVEAGVAAAMKVIDSAEK

SEQ ID NO: 23 - protein, 390 aa, Erwinia toletana
MMRSAFAQPLFTLTTGPVDVYPAVSRALSMPVWYDYDPAFQNCFERVSLKAAQALESATPPLILQGEPVLALEAAAASLLSAEDMVLNLVSGVYAAGFSEWARRYSRQVEELRVEFNQVIDPVAVDAWLSRHPQITVVVVCHHDTPSGTLNPLDEIGRVVKKHGKLLLVDAVSSFAGVAVTAASCQADLFITGPNKCLGCPPGLSLVAVSDAAWRKIEANDAAPRASVLSLLDWRDAWSAEKPFPYTPSVAEINGLEAALDGYLQEGPAQVWARHRLTAQAFRAGIQAMGLSLWAADESIASPTATAIRVPDGVNEAQWRLRARECYGVMFSSGRGETLGKVLRVGHMGPTAQPMFAIIALTALAGALNQLREPQLDIGAGVAAAMAVIG

SEQ ID NO: 24 - protein, 386 aa, Herbiconiux ginsengi
MAHPDFTLSAGPVTVTARTLAGLGSPILYHYDPEFLATFRRTQGKVGQMFQTDNDIILMQGEAIVGLEGALRSLITPGMHVLNLVQGVFGKGTGYWIADFGAVLHEIEVGYDDAVSPAQVEEYLDAHPEIQMVCLVASETPSGTVTDVAAIGPMCRDRGILTYIDTVSGVLGMPWKTDEWGLDLCVAGAQKCLGGPPGVALISVSQKAWDVMYANPAAPRDSYLSLIDWKEKWLGEGRFPYTPSVSDMNGLEAALDQALEEGIDAVVARHEAAAAVTRAGARAMGLELWAKSEEIASACVTSIRLPDDIDNAVVRDHAREVYGVMLSHGQGAGNLVRLSHMGPTAGGLHSVVGLAALGRTLADLGMSVDIGAGLEAALALLSTQRR

SEQ ID NO: 25 - DNA, 1179 bp, Mesorhizobium sp. YR577
atgcgatatcccgaccacactgagccggtgatcacactcaccgccggtcccgtcaacgcctatcccgatgtgctacgggggctcgggcgcacggtgctttacgactacgacccggcgttccagctcttctacgagaaggtcatcggcaaggcgcagaaggccatgcgtctgtcgaataagccggtcgtcctgcatggcgagccggtgatgggtctggaggccgccgcggcgtcgctcatctctagggatgatgtggtgctcaatctggcatcgggtgtctacggcaaggggttcggctattgggcgaagcgttattcgccgaacctgctggagatcgaggttccttacaatgaggcgatcgatccgcagtccgtcgcggacatgctgaaatcgcatcccgaaatcaccgtcgtttcggtctgccatcacgacacgccgtcaggcacgatcaacccgatcgatgagatcggagcgatcgtttcggcgcatggcggctatctgatcgtcgacgccgtttcgtccttcggcggcatgaaaacgcatcccgaggattgcaaggccgacatctatgtgaccgggccgaacaagtgccttggcgcacctcccggactgacgctgatgagcgtgagcgaccgtgcctggaccaggatgaaagcgaactctgcagcacctcgcgcatcggttctgagcattctcgattgggagcatgcatggtccaaggaaaagccgtttccgtttacgccgtccgtcgccgaaatgaacggcctggatgtcgcgctcgacctttatctcaacgaggggccggaagcggtgtgggcgcggcacgccctgaccgccaaggcgacacgcgccggggctcaggcgatgggcctgtcgatctgggctgcaaatgaaaagatcgcctcaccgacaacaaccgccatccgcacgccggacggggtcgatgaaaaggcgctgcgtcaggccgcgcgtgagcgttacggcgtggtgttttcttccggccggggcgaaacgctcggcaaactgacgcgcattggccacatggggccaacggcccagccgatctacgcgatcgcagcgctgacggctttgggcggtgcgctgaatgcggccggccggaagcttcaggttggcaagggtatcgaggcagcactcgccgtgatcgatgcggatgcctga

SEQ ID NO: 26 - DNA, 1179 bp, Pseudaminobacter salicylatoxidans
atgcgatataaagacaatgccgacccggtgatcacgctcacctcggggcccgtcaacgcctatgcggatgtgctgcgcgggctgagccgcactgtgctctacgactatgatccggcgttccagctcttctatgaaaacgtggtggccaaggcgcagaaggccatgcgtctttccacccggccggtgctgttgcagggtgagccggttatgggactggaggcagcggcagcttcactgatttccagcgaggatgtcgtcctgaacctcgcctctggcgtctatggcaagggcttcggctactgggcaaagcgctattcgccgaacctgctcgagatcgaaaccccctataacgaatccatcgacccgcaggcggttgccgatatgcttaagaagcatccggaaatcaccgttgtctcggtgtgccaccacgacacaccgtcgggcacgatcaacccgatcgacgaaattggcgcgatcgtatcggcgcacggggcctatttcatcgtcgatgccgtatcttcattcggaggcatgaagacccatcccgaagactgcaaggccgacctctatgtcactggcccgaacaagtgcctgggcgcgcctccgggactgaccatgatgggcgtcagcgacagggcctgggccaggatgaaagccaatccatccgcaccgcgcgcctcggtgctgagcattctcgactgggaggatgcctggtcgcacgaaaagccgtttccgttcacaccctctgtggcggagatcaacggcctcgacgtcgcgctggacctctatctgaacgaggggccggaagtggtgtgggcgcgccatgcgctgacggcaaaagccacgcgtgcgggcgcattggccatggggttgtcgctctgggcggctagcgagaagattgcctcgcccaccacgacgtcgatccgcacgccgcagggggtggacgaagccgcgcttcggcaggcggtgcgagagcgctatggcgtagtgttttcatccggccgtggcgagacgttgggcaagcttacccgcatcggccacatgggccccacggcgcagccgatttacgcagtcgcggcgctgaccgcgctgggtggcgcgttgaacgcgatgggccagaagctggccgtcggcagaggcatcgacgccgccctcgcggtgatcgacgccgatgcatga

SEQ ID NO: 27 - DNA, 1179 bp, Bauldia litoralis
atgcgctatgcactcgatgccgatcccgtcatcacgctgacctcggggccggtcaacgcctatccggatgtcctgcgggcgttggcgcgcacggttctttacgactacgacccggccttccagctccattacgagcaggtgatcggcaagatgcagacggcgatgcggacgtcgaaccgtccggtcatcctgcatggcgagccggtgctcggactcgaggcggcggcggcctcgctgatcaccgccgacgatactgtgctcaacctggcgtcgggcgtctacggcaaggggttcggctactgggcagcgcgctattcacccaaccagctcgagatcgaggttccctacaaccaggcgatcgacccgcagcaggtcgccgacatgctgaaggccaatccggccatttcggttgtcgccgtctgccatcacgacacgccgtcggggaccatcaacccgatcgacgagataggcgccatcgtcgccgaccatggcgcctacctgatcgtcgacgcggtgtcctccttcggcgggatgaagacccacccggaggattgcaaggccgacatctacgtcaccggcccgaacaaatgcctcggcgcgccgccggccctgaccatgctcggcgtcagcgaacgggcgtggcagaagatgaaggacaatccggccgcgccgcgcgcctcgatgctcagcatcctcgactgggagaatgcctggtcgcgcgacgaggcctttcccttcacgccgtcggtggccgaggtcaacggtctcgacgtcgcgctcgacctctaccttgccgagggaccggaggccgtctgggcgcgccacgcgctgaccgccgcggcgacgcgggccggcatcctggcgatgggcctcgacctgtgggcggcggacgcggccatcgcctcgccgacgacgaccgcggtgcggacgccggagggcatcgatgaagcgaagctgcgcgcgacggtccgcgaacgctatggcgtggtgatgtcgtccgggcgcggcgaaacgctcggcaagctcacgcggatcggccacatgggcccgacggcgcagccgatctatgcgatcgccgcgctgaccgcctttggcggcgctctccaggcgctcggccatcccgcgccggtcggcaaggcgatcgacgcggcgctcgcggtcatcgacgccgccgcctga

SEQ ID NO: 28 - DNA, 1176 bp, Skermanella stibiiresistens
atgacagtcgagcgtatcgtcgagcctgctttcactctcagttccggtcccgtcgacgcgtatcccgccgtgctcagggcactgtcgcggacggtgctctacgacttcgatcccgtgttccagcacttctacgaggcggtgacggataaggccaaggtggcactccgcaccgcggcgccacccgtgatcctccatggcgagccggtgctggggctggaggccgcggccgcctcgctgatcgcccgcgacgatgtcgtgctcaacctagcgtccggcgtctatggcaagggcttcggtttctgggccaaacggtactgcgccgagttggtcgagatcgaggtgccttatgacgacgcgatcgacccggcggcggtcgaggccgccttcagggaacgccccgacatccgtgtggtctcggtctgccatcatgacacgccgtctggcacgctgaacccaatcgacgagatcggccgtatcgtggcggcccatggtgcctatctgatcgtggacgccgtttcctcgttcggggggatggacgtccatcccgaggcctgccaagccgacatcttcgtgacagggccgaacaagtgcctgggctgcccgcctggtctgacgctgctggcggtcagtggccgcgcctgggacaagatgaaggccaatccggacgcgccgcgcgcctcgatcctcagcatcctggattgggagaacgcgtggcggcgtgaccagccgtttcccttcaccccatcgatttccgaagtgaacgcgctggacgcggctctggacctgtacctggaggaggggccggaagccgtctggcgccgtcacgcgctcacatccaaggcctgccgcgacggcatccgcgccatgggcatgacgatatggcccgcgcgggaggcgatcgcctctccgaccaccacggcggtgcgggtgcccgacgggatcgacgcggatgccctgttggcgacggcgcgggatggcttcggcgtcagcttctcgtccgggcggggcgagaccaagggcaagctgatccgcatcggacacatgggccgcaccgcccaaccactctacgccgtcgtcgggctggcggcgctgggcggtgcgttgcggcggctgggagccaatgtcgacgccggcgccggcatggaggcggctctcgccgtgatctcaaaccaataa

SEQ ID NO: 29 - DNA, 1185 bp, Rhizobium sp. AC44/96
atgaaccagacgaaaattgccgatccaatctttaccttgaccacggggccggtcgatgcctatccagcggtgctgcgcgccctgtcccgcccggtgctctacgactacgatccggcgttccttgcgttctacgaagccgtcaatgccaaggtacagcgcgccttccacacgaaaaccccacccgtgatcctccagggcgagcccgtgctggggctcgaagcggctgcggcttcgctgatcgccaagaaagacgtggtgcttaacctcgtttccggcgtctacggcaaggggttcggcttttgggcagcgcgctatgcgcgggagctggtcgaattggaagttccctataacgatgtcctgacggccgatgcggtggccgacatgttgaaaaagcgtccggatatcgccgtggtcgcgctctgccaccatgataccccgtcaggcaccgtcaatcccgtggccgaaatcggtgcagtggtgagagcggcgggaaagctcttcatcgtcgattccgtatcggcctttggcggcatggacgtgcttccggagacggcttgcgccgatattttcgtaaccggccccaataaatgtctcggctgcccgcccggtttgtcgcttctgcacgtcagcgaggctgcctgggagaagatcgccgccaatcccgatgccccgacagcttcgatgctttcgatcagtgattggaaacgagctcacgaggctggccaaccgttcccgtttaccccctcggtttccgaaatcaacgccctcgacgccgccatggatctctatctggaggaaggcgagcaggcagtatgggcgcgccatgcgctgacagccaaggcatgccgggcaggcgtgcaagcagccggcctgaagctctgggcggcacgggaagacatcgcctcgccaacctgcactgccgtggcaatccccgatgggatcgacgaagcaaagctgcgggcgtcgatccgtgatcgctacggcgttgtcttttcttctggccgtgccgagacgctgggcaagcttacgcgcatcgggcatatgggcccgactgcacgtccgacctattcactcgtctcggttgcagccatcgttggcgggctcaaggccgccggcgtcaaggaccttgatgtggaggctggagttgcggcggcaatgaaggtcatagacagtgcagaaaagtga

SEQ ID NO: 30 - DNA, 1173 bp, Erwinia toletana
atgatgcgttccgcctttgcccagccgttatttaccctgactaccggtccggttgatgtctatccggcggtgtcgcgggcattatcgatgccggtctggtacgactacgacccggcttttcagaactgctttgaacgcgtcagcctgaaggccgcacaggcgctggaaagcgccacgccgccgctgatcttacagggtgaaccggtactggcgctggaagccgcggcggcatcgctgcttagcgctgaggatatggtgctgaatctggtttccggcgtatatgccgccggtttctctgagtgggcgcggcgctattcgcgtcaggttgaggagctgcgcgttgaatttaatcaggtgattgatcctgtcgcggttgatgcatggcttagtcgtcatccgcagattaccgtggtggtggtttgccaccatgacacgccgtccggcaccctaaacccactggatgaaattgggcgggtggtgaaaaaacatggcaaattgctgctggtggatgcggtttcttcgtttgccggtgtggcggtgacggcggccagttgccaggcggacctgtttattaccggccccaataaatgtctcggctgtccgccgggcttaagcctggtggcggtatctgacgctgcctggcggaaaatcgaggccaatgacgctgcgccgcgtgcgtcggtactcagcctgctcgactggcgtgatgcctggtcagcggaaaaaccttttccgtataccccatcagtggcggagatcaacgggctggaggcggcgctggacggttatttgcaggaggggccagcgcaggtctgggcgcgccacaggctaaccgcacaggcatttcgcgccgggattcaggcgatgggacttagcctgtgggcggcggatgaaagcattgcttcaccaacggcaactgcgatacgcgtgcccgacggggtaaatgaagcgcaatggcgactgcgcgcgcgtgagtgttatggcgtgatgttttcgtcagggcgcggtgaaacgctgggcaaggtattgcgtgtcggccatatggggccgactgcgcaaccgatgtttgcgattattgcgctgaccgcactggcgggggcacttaatcagctacgcgagccgcagctggatattggcgctggcgtggcggcggcaatggcggtgattggctaa

SEQ ID NO: 31 - DNA, 1161 bp, Herbiconiux ginsengi
atggcacaccccgacttcacgctctcggccgggccggtcaccgtcaccgcccgcacgctcgcgggcctcggctcgccgatcctctaccactacgaccccgaattcctcgcgacgttccggcgcacgcagggcaaggtggggcagatgttccagaccgacaacgacatcatcctcatgcagggtgaggccatcgtcggtctcgagggcgccttgcgctcgctgatcacgcccgggatgcacgtgctgaacctcgtgcagggcgtgttcggcaagggcaccggctactggatcgccgacttcggcgccgtgctgcacgagatcgaggtcggctacgacgacgcggtctcgccggcacaggtcgaggagtacctcgacgcgcatccggagatccagatggtgtgcctggtggcctccgagaccccgtcgggcacggtgacggatgtcgcggcgatcggcccgatgtgccgggaccgcggcatcctgacctacatcgacactgtctcgggcgtgctcggcatgccctggaagaccgacgagtggggcctggacctctgcgtcgccggcgctcagaaatgcctcgggggccctcctggcgtggcgctcatctcggtgagccagaaggcgtgggacgtgatgtacgcgaacccggccgccccgcgtgactcctatctctcgctgatcgactggaaggagaagtggctcggtgagggccgctttccgtacacgccgtcggtgagcgacatgaacgggctcgaagcggcgctcgaccaggcgctggaggaggggatcgacgcggtcgtcgcgcgtcacgaggccgccgccgccgtgacccgcgccggcgcccgagccatgggtctcgagctctgggcgaagagcgaggagatcgcgtcggcctgcgtcacctccatccgcctgcccgacgacatcgacaacgccgtggtgcgcgaccacgcccgcgaggtctacggggtgatgctctcgcacgggcagggtgccggcaacctcgtgcggctctcgcacatgggcccgacagccggtggcctccactcggtggtgggcctcgccgcgctcggccgcaccctcgccgatctgggcatgtccgtcgacatcggagcgggactggaggcggcgctcgcgctgctcagtacccagcggcggtga

SEQ ID NO: 32 - DNA, 1196 bp, Artificial Sequence
  Artificial Fragment for Pyridoxamine Pyruvate Transaminase
acaaaaaggataaaacaatgcgttatcctgatcacactgaaccggtgattacattaaccgcaggtccagttaatgcctatcccgatgtgttacgcggtttgggtcgcaccgtgctttatgattacgacccggcttttcagttattttatgagaaagtcattggtaaagcgcagaaggccatgcgtctgagcaataaaccggttgtcctgcatggcgaaccggtgatgggtctggaggccgcagctgcgtcattgatctctagagatgatgtggtacttaatctggcaagcggtgtttacggtaaagggttcggctattgggctaagcgttattcgcctaacctgttagaaattgaggttccttacaatgaagcgatcgatccgcagtccgtcgctgacatgctgaaaagtcatcccgaaattaccgtagttagtgtctgtcatcacgacactccatcaggtacgatcaatccgattgatgagatcggagcgattgtttcggctcatggcggatatctgatcgtagatgccgttagttcattcggtggcatgaaaacacatcccgaagattgcaaagccgacatatatgtgaccgggccaaacaaatgtcttggcgcacctcccggactgacgctgatgagcgtgagcgaccgtgcctggaccagaatgaaagcgaactctgcagcacctcgcgcaagtgttctgagcattttggattgggagcatgcatggtccaaggaaaaaccgtttccatttactccgtctgtcgccgaaatgaacggactggatgttgctttagacctttatttgaacgaagggcctgaagcggtgtgggctcggcacgcactgaccgccaaagcgacacgcgccggggctcaagcaatgggcttatcgatctgggctgcaaatgaaaagattgcctcaccgacaacaaccgcaatacgcacgccagacggggtcgatgaaaaagcgctgcgtcaggccgctcgtgagcgttacggagtggtattttcttccggccggggagaaactttaggcaaactgacgcgcattggtcacatggggccaactgcgcagccgatctacgcgatagcagcgctgacggctttgggcggtgcgctgaatgccgctggacggaaacttcaagttggcaagggtattgaggcagcattagccgtaatagatgcggatgcctaa

SEQ ID NO: 33 - DNA, 1196 bp, Artificial Sequence
  Artificial Fragment for Pyridoxamine Pyruvate Transaminase
acaaaaaggataaaacaatgcgttataaagataatgccgatccggtgattacactgaccagtggtcctgttaatgcatatgcggatgtgctgcgtggtctgagccgcactgttttatacgactatgatccagcatttcagttattctatgaaaatgtggtggccaaagcacagaaagctatgcgtctttctacccggccggtactgttgcagggtgaacctgttatgggactggaagcagcggcagcttcactgatttccagcgaagatgtcgttctgaacttagcctctggtgtctatggtaagggctttggttactgggcaaaacgctatagtccgaatctgttagagattgaaaccccctataacgaatctatcgacccacaagctgttgccgatatgcttaagaaacatccggaaattaccgttgtctcggtgtgtcatcacgatacacctagtggcacgattaatccgatcgacgaaattggtgcgatagtatcagcacacggggcctattttatcgttgatgctgtatcttcattcggaggcatgaaaacccatcccgaagattgtaaggccgacctttacgttactggtccaaacaaatgcctgggcgcgcctccgggactgacaatgatgggggtcagcgatcgggcctgggccagaatgaaagctaatccatccgcacctcgtgcctcggtgctgagcattttagactgggaggatgcttggagtcatgaaaaaccgtttccattcacaccttctgtagcggagataaatggcttagatgtcgcgctggacctttacctgaacgaggggccggaagtggtgtgggcacgccatgcgctgactgcaaaagccacgcgtgcaggtgcattggctatggggttgtcattatgggcggctagcgagaagattgccagtcccaccacaacgtcgatacgcactccacagggggttgatgaagctgcacttcggcaagcggtgcgtgagcgctatggcgtagtgttttcatccggtcgtggcgagacgttgggtaaacttacccgcatcggacacatgggccccacggcgcagccgatttacgcagttgcggctctgactgcgctgggcggggctttgaacgcgatgggacaaaagctggccgtcggcagaggaatagacgccgctttagcggtaatcgacgccgatgcataa

SEQ ID NO: 34 - DNA, 1196 bp, Artificial Sequence
  Artificial Fragment for Pyridoxamine Pyruvate Transaminase
acaaaaaggataaaacaatgcgttatgcactggatgccgatcctgttattacactgaccagcggtccggttaatgcatatccggatgtgctgcgtgctttggcgcgcacagttctttatgattacgaccctgcttttcagttacattatgaacaggtgatcggtaaaatgcaaacggcaatgcggacaagcaaccgtccggtcattctgcatggcgaaccagtgctgggattagaggcggctgcggccagtctgattaccgcagacgatactgtgttaaatctggcatctggtgtttacggcaaagggtttggttattgggcagcgcgttattcacccaaccagctggaaatcgaggttccctacaaccaggctattgatccgcagcaagtcgccgatatgctgaaggctaatcctgccatttctgttgttgcagtatgtcatcacgacactccgtcagggaccattaatccaatcgatgaaataggtgccattgtcgccgaccatggagcctatctgattgttgatgcggtgagttccttcggcgggatgaaaacccacccggaggattgcaaagcagacatctacgtaacaggtcctaacaaatgtttaggcgcaccgccagccctgaccatgttgggtgtcagcgaaagagcgtggcagaagatgaaagataatccggcagctcctcgcgcctcgatgttaagcattcttgactgggaaaatgcctggtctcgtgatgaggcctttcccttcacgccgtcagtggctgaagttaacgggttagacgtagcgctggatttataccttgccgagggaccagaagcagtctgggcacgccatgcgctgaccgccgctgcgactcgggctggcatactggcaatgggtttggacctgtgggcggctgatgcggccatcgcaagtccgacaacgaccgcagtgagaactcctgagggcattgatgaagcgaaactgcgtgctacagtacgcgaacgttatggagtggtgatgtcgtccgggcgcggcgaaacgttgggtaaacttactcggataggccacatgggaccaacggcgcagcctatctatgcaattgccgcgctgaccgcttttggtggcgctttacaagcgttgggtcatcccgctccagtaggaaaggcaatcgacgcggctcttgcggtcatagatgccgcagcctaa

SEQ ID NO: 35 - DNA, 1193 bp, Artificial Sequence
  Artificial Fragment for Pyridoxamine Pyruvate Transaminase
acaaaaaggataaaacaatgacagttgaacgtattgttgaacctgcttttactttaagtagcggtcctgtcgatgcgtatccagccgtgttacgcgcactgtcacgtacggttttatacgattttgatcctgtgttccagcacttttatgaagcagttactgataaagctaaagtggcacttcgtaccgcggcaccacctgtaatcttgcatggcgaaccggtgctgggtttagaggccgcggctgccagcctgattgcacgcgacgatgttgtacttaatttagcgtctggtgtctatggcaagggtttcggtttttgggccaaacgttactgtgctgaattggttgagattgaagtgccttatgatgacgcgatcgatccggcggcggtcgaggccgcattcagagaacgccccgacattcgtgtagttagtgtctgccatcatgatacgccgtctggcactctgaacccaattgatgaaatcggacgtatagtggcagcccatggtgcttatctgattgtagacgccgtttcttcgtttggtgggatggatgttcatccagaggcttgtcaagccgacatctttgtgacagggccgaataaatgcctgggctgtccgcctggtttgacgctgctggcggtcagtggccgcgcttgggataaaatgaaggccaatccggacgcgccgagagcatcaattcttagcatactggattgggaaaacgcgtggcggcgtgatcagccgtttcccttcaccccaagtatttccgaagtgaatgcactggatgcggctttagacctgtatctggaagaggggccggaagccgtatggcgccgtcacgcacttacatcaaaagcctgccgcgatggcatccgcgctatgggaatgactatatggcccgcgcgtgaggcaattgcttctccgaccaccacggcggtgcgggtacccgacgggattgatgcagatgctctgttggcgacagcacgtgatggctttggtgttagcttctcgtccgggcggggcgaaaccaaaggaaagctgatccgcataggacacatgggtcgcaccgcccagccattatacgccgttgtcgggttagcagcgctgggcggtgcgttgagacggctgggagctaatgtggacgccggcgcaggaatggaggcagctcttgccgtcatctcaaaccaataa

SEQ ID NO: 36 - DNA, 1202 bp, Artificial Sequence
  Artificial Fragment for Pyridoxamine Pyruvate Transaminase
acaaaaaggataaaacaatgaatcagacgaaaattgcagatccaatttttaccttgaccacaggtccggttgatgcctatccagcggtgctgcgtgcactgtctcgcccggtgttatatgactacgatccggcgtttcttgcgttctatgaagccgtcaatgctaaagtacagcgtgcctttcatacgaaaaccccacctgtgatcctgcagggtgaacctgtgctggggttagaagcggctgcggctagcctgattgccaagaaagatgtggtgcttaacctggtttctggcgtttacggtaaagggttcggcttttgggcagcgcgctatgcgcgtgagctggtcgaattggaagttccctataacgatgtcctgacagccgatgcggtagctgacatgttgaaaaagcgtccagatatcgccgtggttgcgttatgtcaccatgataccccgtcaggtaccgtcaatcctgtagccgaaataggtgcagtggtgagagcggcgggaaaactgtttattgttgattccgtaagtgcctttggtggcatggacgtgttaccggaaacggcttgcgctgatattttcgtaactggtcccaataaatgtttaggctgcccgcctggtttgtctcttctgcacgtcagcgaagctgcctgggagaagattgcagccaatcccgatgcaccaacagcttcaatgcttagcatcagtgattggaaaagagctcacgaggctggtcaaccgttcccgtttactccttcggtttccgaaataaacgccctggacgctgccatggatttatatctggaggaaggcgagcaggcagtatgggcgcgccatgcgctgacagccaaagcatgtcgggcaggagtgcaagcagccggcctgaagttatgggcggcacgggaagacatcgcttcaccaacctgcactgccgtggcaattcccgatgggattgacgaagcaaaactgcgggcgagtatacgtgatcgctacggagttgtcttttctagcggccgtgctgagacgctgggtaagttgacacgcatcgggcatatgggcccgactgcacgtcctacctattcattagtctcggttgcagccattgttggagggctgaaagcagccggcgttaaagaccttgatgtagaggctggagttgcggcggcaatgaaggtcatagacagtgcagaaaaataa

SEQ ID NO: 37 - DNA, 1190 bp, Artificial Sequence
  Artificial Fragment for Pyridoxamine Pyruvate Transaminase
acaaaaaggataaaacaatgatgcgttccgcctttgcccagccgttatttaccctgactacaggtcctgttgatgtctatccggcggtgagtcgtgcattatctatgcctgtctggtatgattacgacccggcttttcagaactgctttgaacgcgtcagcctgaaggccgcacaggctctggaaagtgccacgccgcctctgatcttacagggtgaacccgtactggcactggaagccgcagcggcatcactgcttagcgctgaagatatggtgctgaatctggtttccggcgtatatgccgccggtttctctgagtgggcgcggcgttactctcgtcaagttgaagagctgcgcgttgaatttaatcaggtgattgatcctgtcgctgttgatgcatggcttagtcgtcatccgcagattaccgtggttgtggtttgccaccatgatacgccttccggcaccttaaacccactggatgaaattgggcgggttgtgaaaaaacatggtaaattgctgcttgtggatgcggtttcttcgtttgccggtgtggcagtcacagcggccagttgccaagctgacctgttcataaccggccccaataaatgtttaggctgtccgccaggattaagcctggtggcggtatctgatgctgcctggcggaaaatcgaggccaatgacgctgcaccgcgtgcgagcgtattatcgttgttagactggcgtgatgcctggtcagctgaaaaaccttttccctatacaccatcagtagcggagatcaacggactggaagcagcgctggacggttacttgcaggaggggccagctcaggtctgggcgcgccacagattaaccgcacaagcattccgcgccgggattcaggcaatgggacttagcttgtgggcggctgatgaaagtattgcttcaccaacggcaactgcgatacgcgttcccgacggagtaaatgaagcccaatggagactgcgcgcgcgtgagtgttatggcgtgatgttttcgtcagggcgcggtgaaacgctgggcaaggtattgcgtgtcggtcatatgggaccgactgcccaaccaatgttcgcgattatcgctcttacagcactggcgggggcacttaaccagttaagagagccgcaactggatattggcgctggcgttgcagccgcaatggctgtgataggttaa

SEQ ID NO: 38 - DNA, 1178 bp, Artificial Sequence
  Artificial Fragment for Pyridoxamine Pyruvate Transaminase
acaaaaaggataaaacaatggcacatcctgattttacactgagcgccggtccggttaccgttaccgcacgcacgctggcgggtttaggcagtccgattctgtatcattatgaccctgaatttttagcaacatttcgtcgcacgcagggtaaagtggggcagatgttccaaaccgataatgacattatcctgatgcagggtgaagccattgttggtcttgaaggcgctttgcgttctctgataacgcccgggatgcatgttctgaacttagtgcagggagtatttggcaaaggtaccggctactggattgccgatttcggagctgtgctgcacgagatcgaagttggctatgatgacgcggtctcaccggcacaagttgaagagtatcttgatgcacatccagaaattcagatggtttgtctggtggcctccgagaccccgagtggtacagtaacggatgtcgcggcaattggcccaatgtgtcgggatcgtggaatcctgacttatatagatactgtttctggcgtgttgggtatgccctggaaaacagacgaatggggcctggatttatgcgtcgcaggagctcagaaatgtctggggggccctcctggtgttgcgcttatttcagtgagccaaaaggcatgggatgtaatgtacgcgaatccggccgctccacgtgactcatatttaagtctgatcgattggaaagagaaatggctgggtgaaggccgctttccgtatacgccatctgtgagcgacatgaacgggcttgaagcagcgttagatcaggcactggaagaggggattgacgcggttgtcgcacgtcacgaagccgctgccgctgtgacccgcgccggagctagagccatgggtctggaactttgggcgaagagcgaagagatagcatcggcttgcgttacttccattcgtctgcccgatgacatcgataatgccgtggtacgcgatcatgctcgtgaagtctacggggtgatgttatcacacgggcagggtgccggcaacttggtacggttatctcacatgggtccgacagctggtggcctgcattcggtggtaggacttgccgcgttaggccgcaccttggctgacctgggaatgtccgtcgatataggtgcgggactggaggcggcattagcgctgttgagtactcaaagacggtaa

SEQ ID NO: 39 - protein, 6 aa, Artificial Sequence
  Specific Amino Acid Residues
  misc_feature (1)..(1): Xaa is Leu, Met, Ile or Val
  misc_feature (2)..(2): Xaa is His or Gln
  misc_feature (3)..(3): Xaa is Gly, Cys or Ala
  misc_feature (4)..(4): Xaa is Glu or Asp
  misc_feature (5)..(5): Xaa is Pro or Ala
  misc_feature (6)..(6): Xaa is Val, Ile, Leu or Ala
XXXXXX

SEQ ID NO: 40 - protein, 9 aa, Artificial Sequence
  Specific Amino Acid Residues
  misc_feature (1)..(1): Xaa is His or Ser
  misc_feature (2)..(2): Xaa is Glu or Asp
  misc_feature (8)..(8): Xaa is Ile, Val or Leu
  misc_feature (9)..(9): Xaa is Asn or Thr
XXTPSGTXX

SEQ ID NO: 41 - protein, 7 aa, Artificial Sequence
  Specific Amino Acid Residues
  misc_feature (1)..(1): Xaa is Val, Ile or Ala
  misc_feature (3)..(3): Xaa is Ala, Thr or Ser
  misc_feature (6)..(6): Xaa is Ser, Ala or Gly
  misc_feature (7)..(7): Xaa is Phe, Trp or Val
XDXVSXX

SEQ ID NO: 42 - protein, 10 aa, Artificial Sequence
  Specific Amino Acid Residues
  misc_feature (1)..(1): Xaa is Gly or Ser
  misc_feature (2)..(2): Xaa is Pro, Ser or Ala
  misc_feature (3)..(3): Xaa is Asn, Gly, Ser, Ala or Gln
  misc_feature (6)..(6): Xaa is Leu or Met
  misc_feature (8)..(8): Xaa is Ala, Ser, Cys or Gly
  misc_feature (9)..(9): Xaa is Pro, Thr, Ser or Ala
XXXKCXGXXP

SEQ ID NO: 43 - protein, 9 aa, Artificial Sequence
  Specific Amino Acid Residues
  misc_feature (1)..(1): Xaa is Gly or Asp
  misc_feature (2)..(2): Xaa is Val or Ile
  misc_feature (3)..(3): Xaa is Val, Thr, Ala, Ser, Met, Ile or Leu
  misc_feature (4)..(4): Xaa is Phe, Met, Leu, Ile or Val
  misc_feature (6)..(6): Xaa is Ser, Gly, Ala, Thr, Ile, Leu or His
  misc_feature (8)..(8): Xaa is Arg, Met or Gln
  misc_feature (9)..(9): Xaa is Gly, Arg, Ala, Asp, His or Lys
XXXXSXGXX

SEQ ID NO: 44 - protein, 11 aa, Artificial Sequence
  Specific Amino Acid Residues
  misc_feature (1)..(1): Xaa is Leu or Val
  misc_feature (2)..(2): Xaa is Thr, Ile, Val or Leu
  misc_feature (4)..(4): Xaa is Ile, Val or Leu
  misc_feature (5)..(5): Xaa is Gly or Ser
  misc_feature (9)..(9): Xaa is Pro, Ala or Arg
  misc_feature (10)..(10): Xaa is Thr, Val or Ser
XXRXXHMGXXA

SEQ ID NO: 45 - protein, 19 aa, Artificial Sequence
  Specific Amino Acid Residues
  misc_feature (1)..(1): Xaa is Val, Leu, Ile or Met
  misc_feature (2)..(2): Xaa is Ile, Leu or Val
  misc_feature (3)..(3): Xaa is Leu, Met, Ile or Val
  misc_feature (4)..(4): Xaa is His or Gln
  misc_feature (5)..(5): Xaa is Gly, Cys or Ala
  misc_feature (6)..(6): Xaa is Glu or Asp
  misc_feature (7)..(7): Xaa is Pro or Ala
  misc_feature (8)..(8): Xaa is Val, Ile, Ala or Leu
  misc_feature (9)..(9): Xaa is Leu Met, Pro or Val
  misc_feature (10)..(10): Xaa is Gly or Ala
  misc_feature (11)..(11): Xaa is Leu or Ile
  misc_feature (12)..(12): Xaa is Glu or Gln
  misc_feature (13)..(13): Xaa is Ala or Gly
  misc_feature (14)..(14): Xaa is Ala or Val
  misc_feature (15)..(15): Xaa is Ala or Leu
  misc_feature (16)..(16): Xaa is Ala, Leu, His or Tyr
  misc_feature (17)..(17): Xaa is Ser, Gly or Ala
  misc_feature (18)..(18): Xaa is Leu, Phe, Val or Ala
  misc_feature (19)..(19): Xaa is Ile, Phe, Val or Leu
XXXXXXXXXXXXXXXXXXX

SEQ ID NO: 46 - protein, 21 aa, Artificial Sequence
  Specific Amino Acid Residues
  misc_feature (1)..(1): Xaa is Val, Ile, Leu or Met
  misc_feature (2)..(2): Xaa is Val or Ile
  misc_feature (3)..(3): Xaa is Ser, Ala, Val, Cys or Phe
  misc_feature (4)..(4): Xaa is Val, Ile, Ala, Leu or Thr
  misc_feature (5)..(5): Xaa is Cys or Val
  misc_feature (6)..(6): Xaa is His, Asn or Ala
  misc_feature (7)..(7): Xaa is His or Ser
  misc_feature (8)..(8): Xaa is Asp or Glu
  misc_feature (14)..(14): Xaa is Ile, Val or Leu
  misc_feature (15)..(15): Xaa is Asn or Thr
  misc_feature (16)..(16): Xaa is Pro or Asp
  misc_feature (17)..(17): Xaa is Ile, Val, Leu or Ala
  misc_feature (18)..(18): Xaa is Asp, Asn, Glu, Ala, Gly, Gln, Val, Arg or Pro
  misc_feature (19)..(19): Xaa is Ala, Glu, Gln or Asp
  misc_feature (20)..(20): Xaa is Ile or Leu
  misc_feature (21)..(21): Xaa is Gly or Ala
XXXXXXXXTPSGTXXXXXXXX

SEQ ID NO: 47 - protein, 15 aa, Artificial Sequence
  Specific Amino Acid Residues
  misc_feature (1)..(1): Xaa is Gly, Asp or Ala
  misc_feature (2)..(2): Xaa is Ala, Gly, Lys, Thr, Gln, Arg or Glu
  misc_feature (3)..(3): Xaa is Tyr, Asn, Leu or Phe
  misc_feature (4)..(4): Xaa is Leu, Phe, Met or Val
  misc_feature (5)..(5): Xaa is Ile, Leu or Tyr
  misc_feature (6)..(6): Xaa is Val, Ala or Ile
  misc_feature (8)..(8): Xaa is Ala, Ser or Thr
  misc_feature (11)..(11): Xaa is Ser, Ala or Gly
  misc_feature (12)..(12): Xaa is Phe, Trp or Val
  misc_feature (13)..(13): Xaa is Gly, Ala or Leu
  misc_feature (14)..(14): Xaa is Gly or Ser
  misc_feature (15)..(15): Xaa is Met, Val or Leu
XXXXXXDXVSXXXXX

SEQ ID NO: 48 - protein, 24 aa, Artificial Sequence
  Specific Amino Acid Residues
  misc_feature (1)..(1): Xaa is Ala, Ser, Val or Ile
  misc_feature (2)..(2): Xaa is Asp, Gly or Ala
  misc_feature (3)..(3): Xaa is Ile, Leu, Phe, Val or Met
  misc_feature (4)..(4): Xaa is Tyr, Phe, Leu or Cys
  misc_feature (5)..(5): Xaa is Val or Ile
  misc_feature (6)..(6): Xaa is Thr or Ala
  misc_feature (7)..(7): Xaa is Gly or Ser
  misc_feature (8)..(8): Xaa is Pro, Ser or Ala
  misc_feature (9)..(9): Xaa is Asn, Gly, Ser, Gln or Ala
  misc_feature (12)..(12): Xaa is Leu or Met
  misc_feature (14)..(14): Xaa is Ala, Ser, Cys or Gly
  misc_feature (15)..(15): Xaa is Pro, Thr, Ser or Ala
  misc_feature (17)..(17): Xaa is Gly, Ala or Ser
  misc_feature (18)..(18): Xaa is Leu or Val
  misc_feature (19)..(19): Xaa is Thr, Ser or Ala
  misc_feature (20)..(20): Xaa is Met, Ile, Leu, Val or Phe
  misc_feature (21)..(21): Xaa is Met, Leu, Val, Ala or Ile
  misc_feature (22)..(22): Xaa is Gly, Ala, His or Ser
  misc_feature (23)..(23): Xaa is Val, Ile or Ala
XXXXXXXXXKCXGXXPXXXXXXXS

SEQ ID NO: 49 - protein, 10 aa, Artificial Sequence
  Specific Amino Acid Residues
  misc_feature (1)..(1): Xaa is Tyr, Phe, His or Ser
  misc_feature (2)..(2): Xaa is Gly or Asp
  misc_feature (3)..(3): Xaa is Val or Ile
  misc_feature (4)..(4): Xaa is Val, Thr, Ala, Ser, Met, Ile or Leu
  misc_feature (5)..(5): Xaa is Phe, Met, Leu, Ile or Val
  misc_feature (7)..(7): Xaa is Ser, Gly, Ala, Thr, Ile, Leu or His
  misc_feature (9)..(9): Xaa is Arg, Met or Gln
  misc_feature (10)..(10): Xaa is Gly, Arg, Ala, Asp, His or Lys
XXXXXSXGXX

SEQ ID NO: 50 - protein, 16 aa, Artificial Sequence
  Specific Amino Acid Residues
  misc_feature (1)..(1): Xaa is Leu, Gln, Lys, Ala, Phe, Tyr or Trp
  misc_feature (2)..(2): Xaa is Gly, Asn, His or Asp
  misc_feature (3)..(3): Xaa is Lys, Arg or Asn
  misc_feature (4)..(4): Xaa is Leu or Val
  misc_feature (5)..(5): Xaa is Thr, Ile, Val or Leu
  misc_feature (7)..(7): Xaa is Ile, Val or Leu
  misc_feature (8)..(8): Xaa is Gly or Ser
  misc_feature (12)..(12): Xaa is Pro, Ala or Arg
  misc_feature (13)..(13): Xaa is Thr, Val or Ser
  misc_feature (15)..(15): Xaa is Gln, Arg, Glu, Lys, His, Tyr or Gly
  misc_feature (16)..(16): Xaa is Pro or Gly
XXXXXRXXHMGXXAXX

SEQ ID NO: 51 - DNA, 29 bp, Artificial
  Primer Sequence
ccggaattcacaaaaaggataaaacaatg

SEQ ID NO: 52 - DNA, 28 bp, Artificial
  Primer Sequence
cgcggatcctcacgcatccgcgtcgata

SEQ ID NO: 53 - DNA, 1055 bp, Artificial
  Artificial Fragment for Pyridoxine Dehydrogenase
acaaaaaggataaaacaatgtcagtagctgatctaaagaacaacatacacaaactagatacaggatatgggctaatgagcctgacctggcgtgcggaaccgatcccgcagagccaagcgttcgaggcgatgcaccgtgtggttgagctgagccgtgaacgtggtcacaaggcgttctttaacgtgggcgagttctacggcccggactttatcaacctgagctacgttcacgatttctttgcgaagtatccggacctgcgtaaagatgtggttattagctgcaagggtggcgcggacaacgcgaccctgaccccgcgtggcagccacgatgatgtggttcagagcgtgaaaaacagcgttagcgcgatcggtggctatatcgacattttcgaggtggcgcgtattgataccagcctgtgcaccaaaggtgaagtttacccgtatgagagctttgaagcgctggcggagatgatcagcgaaggcgtgatcggtggcattagcctgagcgaggttaacgaggaacaaatccgtgcgattcacaaggactggggtaaattcctgacctgcgttgaggtggaactgagcctgtttagcaacgatatcctgcacaacggtattgcgaagacctgcgcggaactgggcctgagcatcatttgctacagcccgctgggtcgtggcctgctgaccggtcagctgaaaagcaacgcggacatcccggagggcgatttccgtaagagcctgaaacgttttagcgacgaaagcctgaagaaaaacctgaccctggtgcgtttcctgcaagaggaaatcgttgataagcgtccgcagaacaacagcattaccctggcgcaactggcgctgggttgggtgaagcactggaacaaagttccggagtatagcggtgcgaagtttatcccgattccgagcggcagcagcattagcaaagtgaacgagaacttcgacgaacagaagaccaaactgaccgatcaagaatttaatgcgattaacaaatacctgaccaccttccacaccgttggcgaccgctatgagatggcgtaa

SEQ ID NO: 54 - DNA, 34 bp, Artificial
  Primer Sequence
acgcgtcgacacaaaaaggataaaacaatgtcag

SEQ ID NO: 55 - DNA, 29 bp, Artificial
  Primer Sequence
cccaagcttttacgccatctcatagcggt

SEQ ID NO: 56 - DNA, 1133 bp, Artificial
  Artificial Fragment for Alanine Dehydrogenase
acaaaaaggataaaacaatgataataggagtacccacagaaataaagaaccacgaatacagggttgggatggtaccgagcagcgtgcgtgaactgaccatcaagggccacgtggtttacgttcagagcgatgcgggcgtgggtattggtttcaccgaccaagattatatcgatgcgggtgcgagcattctggcgaccgcggcggaagtgtttgcgaaaagcgacatgatcgttaaggtgaaagaaccgcaggcggtggaacgtgcgatgctgcgtcacgaccaaattctgttcacctacctgcacctggcgccggatctgccgcagaccgaggaactgatcaccagcggtgcggtttgcattgcgtatgagaccgtgaccgatgatcgtggtggcctgccgctgctggcgccgatgagcgaggttgcgggtcgtatgagcatccaagcgggtgcgcgtgcgctggagaagagcctgggtggtcgtggtatgctgctgggtggcgttccgggcgtggaaccggcgaaggtggttatcattggtggcggtatggttggtaccaacgcggcgcagatggcggtgggtatgggtgcggatgtggttgtgctggcgcgtagcattgatgcgctgcgtcgtctgaacgttcaattcggcagcgcggtgaaagcgatctacagcaccgcggacgcgattgagcgtcacgttctggaagcggatctggtgatcggcggtgttctggttccgggtgcggcggcgccgaagctgattacccgtgacatggttaagcgtatgaaaccgggcagcgcgatcgttgacgtggcgattgatcagggcggttgcgttgaaaccagccacgcgaccacccaccaagatccgacctacatcgtggacgatgttgtgcactattgcgttgcgaacatgccgggtgcggtggcgcgtaccagcacctttgcgctgaacaacgcgaccctgccgtacatcattaagctggcgaaccagggctataaacaagcgctgctgaacgataagcacctgctgaacggtctgaacgtgatgcacggcaaggttgtgtgcaaggaagtggcggaggcgctgaacctggagtttaccgagccgaagagcctgctggcgtaa

SEQ ID NO: 57 - DNA, 29 bp, Artificial
  Primer Sequence
cgcggatccacaaaaaggataaaacaatg

SEQ ID NO: 58 - DNA, 30 bp, Artificial
  Primer Sequence
acgcgtcgacttacgccagcaggctcttcg

(19) United States
(12) Patent Application Publication
(10) Pub. No.: US 2020/0149079 A1
(43) Pub. Date: May 14, 2020

(54) RECOMBINANT MICROORGANISM AND METHOD OF PRODUCING PYRIDOXAMINE OR SALT THEREOF USING RECOMBINANT MICROORGANISM

(71) Applicants: Mitsui Chemicals, Inc., Minato-ku, Tokyo (JP); RENASCIENCE CO., LTD, Chuo-ku, Tokyo (JP)

(72) Inventors: Toshihiro TATENO, Mobara-shi, Chiba (JP); Tomonori HIDESAKI, Kawasaki-shi, Kanagawa (JP); Tadashi ARAKI, Chosei-gun, Chiba (JP); Atsunori SHINDO, Kawasaki-shi, Kanagawa (JP); Yoshiko MATSUMOTO, Mobara-shi, Chiba (JP); Toshio MIYATA, Nagoya-shi, Aichi (JP); Masaru HONJO, Mobara-shi, Chiba (JP)

(73) Assignees: Mitsui Chemicals, Inc., Minato-ku, Tokyo (JP); RENASCIENCE CO., LTD, Chuo-ku, Tokyo (JP)

(21) Appl. No.: 16/611,064

(22) PCT Filed: May 11, 2018
(86) PCT No.: PCT/JP2018/018412

§ 371 Date: Nov. 5, 2019

(30) Foreign Application Priority Data
May 12, 2017 (JP) 2017-095572

(51) Int. Cl. C12P 17/12; C12N 9/02

(52) U.S. Cl. CPC C12P 17/12; C12R 1/19; C12N 9/0004

(57) ABSTRACT

Provided are a recombinant microorganism comprising: a gene encoding a pyridoxine oxidase; and a gene encoding a pyridoxamine synthetase having an enzymatic activity of synthesizing pyridoxamine from pyridoxal, in which each of the gene encoding a pyridoxine oxidase and the gene encoding a pyridoxamine synthetase is introduced from outside of a bacterial cell, or is endogenous to the bacterial cell and has an enhanced expression, and a method of producing pyridoxamine or a salt thereof from pyridoxine or a salt thereof using the recombinant microorganism.

### TECHNICAL FIELD

The present disclosure relates to a recombinant microorganism that can produce pyridoxamine or a salt thereof, and a method of producing pyridoxamine or a salt thereof using the recombinant microorganism.

### BACKGROUND ART

Pyridoxamine and a salt thereof belong to vitamin B₆ family, and are known to have a glycation reaction inhibitory action. Pyridoxamine and a salt thereof, for example, inhibit accumulation of advanced glycation end products (AGEs) in the body which is involved in various aging processes. AGE is a generic term for substances produced by glycation of proteins, and AGE accumulation is believed to aggravate a disease such as diabetes mellitus, atherosclerosis, chronic renal failure, or Alzheimer's dementia. For this reason, pyridoxamine and a salt thereof are promising substances that are expected to make it possible to prevent or treat such a disease by preventing AGE accumulation.

Pyridoxamine and a salt thereof are also known to have activity as a schizophrenia drug, and various studies for their practical use have been made. In addition, development of health foods and cosmetics utilizing various physiological activities of pyridoxamine and a salt thereof is also in progress.

Pyridoxamine and a salt thereof can be synthesized chemically. For example, International Publication No. WO 2006/066806 discloses a method of chemically synthesizing pyridoxamine dihydrochloride using alanine and formic acid as starting materials. WO 2005/077902 discloses a method of chemically synthesizing pyridoxamine from pyridoxine.

On the other hand, biological synthesis of pyridoxamine is also being studied. WO 2007/142222 discloses a method of obtaining pyridoxamine from pyridoxal using a specific microorganism such as Achromobacter. WO 2007/142222 also discloses an experiment in which, by culturing Acremonium fusidioides in the presence of pyridoxine, a certain amount of the pyridoxine was converted to pyridoxal.

Japanese Patent Application Laid-Open (JP-A) No. H09-107985 discloses a method of producing vitamin B₆, in which a microorganism belonging to the genus Rhizobium and capable of producing vitamin B₆ is cultured under aerobic conditions in a culture medium, and produced vitamin B₆ is obtained from a culture solution.

As a study on vitamin B₆ synthesis, WO 2004/035010 discloses a method of producing vitamin B₆ by culturing an organism in which at least one of the activity of yaaD or yaaE of Bacillus subtilis is enhanced compared to the parent organism. Japanese Patent Application Laid-Open (JP-A) No. 2000-23690 describes a production of a vitamin B₆ mixture by using cell-free extract derived from Rhizobium meliloti IFO 14782. Journal of Molecular Catalysis B: Enzymatic, 2010, vol. 67, p. 104-110 discloses a pyridoxamine-pyruvate aminotransferase (PPAT) that is enabled to use L-glutamate by modifying its sequence. It is described that pyridoxamine was able to be produced by incubating E. coli expressing PPAT having such an modified sequence in the presence of a saturated amount of pyridoxal.

### SUMMARY OF INVENTION

### Technical Problem

However, high reaction yields have not been achieved by pyridoxamine synthesis by chemical synthesis. For example, in a method described in WO 2006/066806, pyridoxamine dihydrochloride is synthesized through many chemical reactions, which results in lower reaction yields. In a method described in WO 2005/077902, a dimer and a trimer of pyridoxamine are produced, and the reaction yield is not significantly high.

On the other hand, in a method described in WO 2007/142222, in which a specific kind of microorganism is used, the conversion efficiency from pyridoxal to pyridoxamine is varied widely among microbial species and is not significantly high in all. In a culture experiment of Acremonium fusidioides in the presence of pyridoxine described in WO 2007/142222, most of a product is pyridoxal rather than pyridoxamine. JP-A No. H09-107985 describes that most of produced vitamin B₆ was pyridoxol (pyridoxine), and production of pyridoxamine was slight.

WO 2004/035010 describes that vitamin B₆ is obtained by culturing in a culture medium a microorganism highly expressing the genes for yaaD and yaaE; however, the product is a mixture of pyridoxine, pyridoxal, pyridoxamine, pyridoxamine phosphate, and the like and pyridoxamine is not selectively obtained.

As described above, chemical synthesis of pyridoxamine or a salt thereof (such as WO 2006/066806 or WO 2005/077902) involves many reaction and purification steps, and high yields have not been achieved. Although some microorganisms have pyridoxamine-synthesizing ability (for example, WO 2007/142222 and JP-A No. H09-107985), it takes time and effort to newly discover such microorganisms. In addition, selective synthesis of pyridoxamine or a salt thereof among the vitamin B₆ family has not been realized and high pyridoxamine production efficiency has not been achieved. The approach of screening specific microbial species from naturally occurring microbial species as described above does not provide molecular biological information as to which enzyme or gene is important for a high production of vitamin B₆.

For example, a method of producing vitamin B₆ using recombinant microorganisms produced by genetic modification technology is described (WO 2004/035010 and Journal of Molecular Catalysis B: Enzymatic, 2010, vol. 67, p. 104-110). However, in production of substances, vitamin B₆ is non-selectively obtained using a common culture medium (WO 2004/035010), or alternatively, a saturated amount of pyridoxal, which is an expensive raw material, is used to produce pyridoxamine (Journal of Molecular Catalysis B: Enzymatic, 2010, vol. 67, p. 104-110). Therefore, inexpensive and selective production of pyridoxamine or a salt thereof has not been achieved.

In view of the above, the disclosure provides a recombinant microorganism capable of inexpensively producing pyridoxamine or a salt thereof from pyridoxine or a salt thereof at high production efficiency, and a method of inexpensively producing pyridoxamine or a salt thereof from pyridoxine or a salt thereof at high production efficiency using the recombinant microorganism.

### Solution to Problem

The disclosure includes the following aspects.

<1>

A recombinant microorganism, including:

a gene encoding a pyridoxine oxidase, and a gene encoding a pyridoxamine synthetase having an enzymatic activity of synthesizing pyridoxamine from pyridoxal,

wherein each of the gene encoding the pyridoxine oxidase and the gene encoding the pyridoxamine synthetase is introduced from outside of a bacterial cell, or is endogenous to the bacterial cell and has an enhanced expression.

<2>

The recombinant microorganism according to <1>, wherein the pyridoxamine synthetase is a pyridoxamine-pyruvate transaminase, a pyridoxamine-oxaloacetate transaminase, an aspartate transaminase, or a pyridoxamine phosphate transaminase.

<3>

The recombinant microorganism according to <1> or <2>, wherein the pyridoxine oxidase is represented by the enzyme number EC 1.1.3.12.

<4>

The recombinant microorganism according to any one of <1> to <3>, wherein the gene encoding the pyridoxine oxidase is derived from Microbacterium luteolum.

<5>

The recombinant microorganism according to any one of <1> to <4>, wherein the gene encoding a pyridoxine oxidase:

(a) has a nucleotide sequence of SEQ ID NO:5,

(b) has a nucleotide sequence that hybridizes with DNA having a nucleotide sequence complementary to the nucleotide sequence of SEQ ID NO:5 under a stringent condition, and that encodes a protein having pyridoxine oxidase activity,

(c) has a nucleotide sequence encoding a protein that has an amino acid sequence of SEQ ID NO:1, or

(d) has a nucleotide sequence encoding a protein that has an amino acid sequence having 80% or more sequence identity with the amino acid sequence of SEQ ID NO:1 and that has pyridoxine oxidase activity.

<6>

The recombinant microorganism according to any one of <1> to <5>, wherein the pyridoxamine synthetase includes at least one of the following partial amino acid sequence (c), partial amino acid sequence (d), partial amino acid sequence (e), partial amino acid sequence (f), partial amino acid sequence (g), or partial amino acid sequence (h), and has an enzymatic activity of synthesizing pyridoxamine from pyridoxal:

(c) X₈X₉X₁₀X₁₁X₁₂X₁₃ (SEQ ID NO:39)

wherein X₈ represents L, M, I or V,

X₉ represents H or Q,

X₁₀ represents C or A,

X₁₁ represents E or D,

X₁₂ represents P or A, and

X₁₃ represents V, I, L or A;

(d) X₁₄X₁₅TPSGTX₁₆X₁₇ (SEQ ID NO:40)

wherein X₁₄ represents H or S,

X₁₅ represents D or E,

X₁₆ represents I, V, or L, and

X₁₇ represents N or T;

(e) X₁₈DX₁₉ vSX₂₀X₂₁ (SEQ ID NO:41)

wherein X₁₅ represents V, I, or A,

X₁₉ represents A, T, or S,

X₂₀ represents S, A, or G, and

X₂₁ represents F, W, or V;

(f) X₂₂X₂₃X₂₄KCX₂₅GX₂₆X₂₇P (SEQ ID NO:42)

wherein X₂₂ represents G or S,

X₂₃ represents P, S, or A,

X₂₄ represents N, S, A, or Q,

X₂₅ represents L or M,

X₂₆ represents A, S, C, or G, and

X₂₇ represents P, T, S, or A;

(g) X₂₈X₂₉X₃₀X₃₁SX₃₂GX₃₃X₃₄ (SEQ ID NO:43)

wherein X₂₈ represents G or D,

X₂₉ represents V or I,

X₃₀ represents V, T, A, S, M, I, or L,

X₃₁ represents F, M, L, I, or V,

X₃₂ represents S, A, T, I, L, or H,

X₃₃ represents R, M, or Q, and

X₃₄ represents R, A, D, H, or K; and

(h) X₃₅X₃₆RX₃₇X₃₈HMGX₃₉X₄₀A (SEQ ID NO:44)

wherein X₃₅ represents L or V,

X₃₆ represents T, I, V, or L,

X₃₇ represents I, V, or L,

X₃₈ represents G or S,

X₃₉ represents P, A, or R, and

X₄₀ represents T, V, or S.

<7>

The recombinant microorganism according to any one of <1> to <6>, wherein the pyridoxamine synthetase is represented by the enzyme number EC 2.6.1.30.

<8>

The recombinant microorganism according to any one of <1> to <7>, wherein the gene encoding a pyridoxamine synthetase is derived from Mesorhizobium loti.

<9>

The recombinant microorganism according to any one of <1> to <8>, wherein the gene encoding a pyridoxamine synthetase:

(a) has a nucleotide sequence of any one of SEQ ID NO:6 or SEQ ID NO:25 to SEQ ID NO:31, or

has a region between an 18th nucleotide and a 3′ end in a nucleotide sequence of SEQ ID NO:10, or a region between an 18th nucleotide and a 3′ end in a nucleotide sequence of any one of SEQ ID NO:32 to SEQ ID NO:38;

(b) has a nucleotide sequence that hybridizes with DNA having a nucleotide sequence complementary to a nucleotide sequence of any one of SEQ ID NO:6 or SEQ ID NO:25 to SEQ ID NO:31, or with DNA having a nucleotide sequence complementary to a region between a 18th nucleotide and a 3′ end in a nucleotide sequence of SEQ ID NO:10 or a region between a 18th nucleotide and a 3′ end in a nucleotide sequence of any one of SEQ ID NO:32 to SEQ ID NO:38 under a stringent condition, and that encodes a protein having an enzymatic activity of synthesizing pyridoxamine from pyridoxal;

(c) has a nucleotide sequence encoding a protein that has an amino acid sequence of any one of SEQ ID NO:2 or SEQ ID NO:18 to SEQ ID NO:24; or

(d) has a nucleotide sequence encoding a protein that has an amino acid sequence having 80% or more sequence identity with at least one amino acid sequence selected from the group consisting of SEQ ID NO:2 and SEQ ID NO:18 to SEQ ID NO:24, and that has an enzymatic activity of synthesizing pyridoxamine from pyridoxal.

<10>

The recombinant microorganism according to any one of <1> to <9>, wherein the gene encoding a pyridoxamine synthetase:

(a) has a nucleotide sequence of SEQ ID NO:6;

(b) has a nucleotide sequence that hybridizes with DNA having a nucleotide sequence complementary to the nucleotide sequence of SEQ ID NO:6 under a stringent condition, and that encodes a protein having an enzymatic activity of synthesizing pyridoxamine from pyridoxal;

(c) has a nucleotide sequence encoding a protein that has an amino acid sequence of SEQ ID NO:2; or

(d) has a nucleotide sequence encoding a protein that has an amino acid sequence having 80% or more sequence identity with the amino acid sequence of SEQ ID NO:2, and that has an enzymatic activity of synthesizing pyridoxamine from pyridoxal.

<11>

The recombinant microorganism according to any one of <1> to <5>, wherein the pyridoxamine synthetase is represented by the enzyme number EC 2.6.1.31 or EC 2.6.1.1.

<12>

The recombinant microorganism according to any one of <1> to <5> or <11>, wherein the gene encoding a pyridoxamine synthetase is derived from Escherichia coli.

<13>

The recombinant microorganism according to any one of <1> to <5> or <11> to <12>, wherein the gene encoding the pyridoxamine synthetase:

(a) has a nucleotide sequence of SEQ ID NO:8;

(b) has a nucleotide sequence that hybridizes with DNA having a nucleotide sequence complementary to the nucleotide sequence of SEQ ID NO:8 under a stringent condition, and that encodes a protein having an enzymatic activity of synthesizing pyridoxamine from pyridoxal;

(c) has a nucleotide sequence encoding a protein that has an amino acid sequence of SEQ ID NO:4; or

(d) a nucleotide sequence encoding a protein that has an amino acid sequence having 80% or more sequence identity with the amino acid sequence of SEQ ID NO:4, and that has an enzymatic activity of synthesizing pyridoxamine from pyridoxal.

<14>

The recombinant microorganism according to any one of <1> to <13>, further including a gene encoding a hydrogen peroxide-degrading enzyme that has an enzymatic activity of generating oxygen from hydrogen peroxide.

<15>

The recombinant microorganism according to <14>, wherein the gene encoding the hydrogen peroxide-degrading enzyme is introduced from outside of a bacterial cell, or is endogenous to a bacterial cell and has an enhanced expression.

<16>

The recombinant microorganism according to <14> or <15>, wherein the hydrogen peroxide-degrading enzyme is represented by the enzyme number EC 1.11.1.6.

<17>

The recombinant microorganism according to any one of <14> to <16>, wherein the gene encoding a hydrogen peroxide-degrading enzyme:

(a) has a nucleotide sequence of SEQ ID NO:7;

(b) hybridizes with DNA having a nucleotide sequence complementary to the nucleotide sequence of SEQ ID NO:7 under a stringent condition, and has an enzymatic activity of generating oxygen from hydrogen peroxide;

(c) has a nucleotide sequence encoding a protein that has an amino acid sequence of SEQ ID NO:3; or

(d) has a nucleotide sequence encoding a protein that has an amino acid sequence having 80% or more sequence identity with the amino acid sequence of SEQ ID NO:3, and that has an enzymatic activity of generating oxygen from hydrogen peroxide.

<18>

The recombinant microorganism according to any one of <1> to <17>, including a recombinant E. coli.

<19>

A method of producing pyridoxamine or a salt thereof, the method including bringing the recombinant microorganism according to any one of <1> to <18>, a culture of the recombinant microorganism, or a treated product of the recombinant microorganism or the culture, into contact with pyridoxine or a salt thereof to produce pyridoxamine or a salt thereof in the presence of oxygen.

<20>

The production method according to <19>, wherein the recombinant microorganism, the culture of the recombinant microorganism, or the treated product of the recombinant microorganism or the culture, includes the pyridoxine oxidase and the pyridoxamine synthetase.

<21>

The production method according to <20>, wherein the recombinant microorganism, the culture of the recombinant microorganism, or the treated product of the recombinant microorganism or the culture, further includes a hydrogen peroxide-degrading enzyme.

<22>

The production method according to any one of <19> to <21>, wherein the treated product of the recombinant microorganism or the culture is a product treated by treatment including one or more selected from the group consisting of heat treatment, cooling treatment, mechanical destruction of a cell, ultrasonic treatment, freeze-thaw treatment, drying treatment, pressurized or reduced pressure treatment, osmotic pressure treatment, cell autolysis, surfactant treatment, enzyme treatment, cell separation treatment, purification treatment, and extraction treatment.

<23>

The production method according to any one of <19> to <22>, the method including either or both of the following (A) and (B):

(A) adding pyridoxine or a salt thereof continuously or in several batches to a solution including the recombinant microorganism, or the culture of the recombinant microorganism, or the treated product of the recombinant microorganism or the culture; and

(B) controlling a molar concentration of an amino acid consumed by the pyridoxamine synthetase so as to be 1 or more times a molar concentration of pyridoxine or a salt thereof, in a solution including the recombinant microorganism, the culture of the recombinant microorganism, or the treated product of the recombinant microorganism or the culture.

<24>

The production method according to <23>, wherein the amino acid consumed by the pyridoxamine synthetase is L-alanine, D-alanine, L-glutamic acid, or D-glutamic acid.

### Advantageous Effects of Invention

According to the disclosure, a recombinant microorganism capable of inexpensively producing pyridoxamine or a salt thereof from pyridoxine or a salt thereof at high production efficiency, and a method of inexpensively producing pyridoxamine or a salt thereof from pyridoxine or a salt thereof at high production efficiency using the recombinant microorganism are provided.

### BRIEF DESCRIPTION OF DRAWINGS

FIG. 1 shows results of measurement of pyridoxine hydrochloride concentration, pyridoxal hydrochloride concentration, and pyridoxamine dihydrochloride concentration in Test 7.

FIG. 2 shows results of measurement of pyridoxine hydrochloride concentration, pyridoxal hydrochloride concentration, and pyridoxamine dihydrochloride concentration in Test 8.

FIG. 3 shows results of measurement of pyridoxine hydrochloride concentration, pyridoxal hydrochloride concentration, and pyridoxamine dihydrochloride concentration in Test 9.

FIG. 4-1 shows alignment of sequences of SEQ ID NO:2 and SEQ ID NO:18 to SEQ ID NO:24.

FIG. 4-2 shows alignment of sequences of SEQ ID NO:2 and SEQ ID NO:18 to SEQ ID NO:24.

### DESCRIPTION OF EMBODIMENTS

The disclosure provides a recombinant microorganism (hereinafter referred to as a recombinant microorganism according to the disclosure), including:

a gene encoding a pyridoxine oxidase; and a gene encoding a pyridoxamine synthetase having an enzymatic activity of synthesizing pyridoxamine from pyridoxal,

wherein each of the gene encoding the pyridoxine oxidase and the gene encoding the pyridoxamine synthetase is introduced from outside of a bacterial cell, or is endogenous to the bacterial cell and has an enhanced expression. In the disclosure, “introduction” refers to introduction in such a manner to allow expression in a bacterial cell.

Until now, it has not been known a method for inexpensively producing pyridoxamine or a salt thereof from pyridoxine or a salt thereof at high production efficiency either by a chemical method or a biological method. The structure of pyridoxamine is shown below.

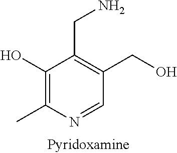

However, the present inventors have surprisingly found that pyridoxamine or a salt thereof can be inexpensively produced from pyridoxine or a salt thereof at high production efficiency by using a recombinant microorganism having above described constitution or a culture of the recombinant microorganism, or a treated product of the recombinant microorganism or the culture. Although the reason for this is not necessarily clear, it is assumed that since each of the genes encoding the above two kinds of enzymes is introduced from outside of a bacterial cell, or is endogenous to the bacterial cell and has an enhanced expression, the enzyme can be expressed at a high expression level from the introduced or enhanced gene. Further, it is assumed that due to the cooperative action of the above two kinds of enzymes, equilibrium of each reaction in a process of producing pyridoxamine or a salt thereof from pyridoxine or a salt thereof and the amount of a by-product or the like produced in this process or the amount of a raw material or the like consumed in this process advantageously affect the production of pyridoxamine or a salt thereof from pyridoxine or a salt thereof.

Furthermore, in a case in which the recombinant microorganism, the culture of the recombinant microorganism, or the treated product of the recombinant microorganism or the culture according to the disclosure is used, it is assumed that pyridoxal is temporarily produced as an intermediate product. However, since pyridoxal is an aldehyde and is highly reactive, pyridoxal spontaneously reacts with an amino group donor such as alanine at a near-neutral pH even in the absence of a catalyst such as an enzyme to produce pyridoxamine and a by-product. In a spontaneous reaction of pyridoxal, it is assumed that the production efficiency of pyridoxamine is not to be high because a by-product is produced and the selectivity of pyridoxamine production is not high. In contrast, in a case in which the recombinant microorganism, the culture of the recombinant microorganism, or the treated product of the recombinant microorganism or the culture according to the disclosure is used, pyridoxal an intermediate is rapidly converted to pyridoxamine by a pyridoxamine synthetase, accumulation of pyridoxal is suppressed, and production of a by-product is also suppressed. As a result, high pyridoxamine production efficiency can be achieved.

Such a cooperative action of the above-described two kinds of enzymes, which has not been found so far, allows the production of pyridoxamine or a salt thereof with high production efficiency by using pyridoxine or a salt thereof, without the need to carry out a multi-step reaction as in a chemical synthesis method. Moreover, by using the recombinant microorganism according to the disclosure, it is possible to avoid by-production of large amounts of other substances included in the vitamin B₆ complex. Pyridoxine is a raw material which is industrially available at a low cost compared to pyridoxal. By using pyridoxine as a starting material, pyridoxamine or a salt thereof can be inexpensively produced.

### <Pyridoxine Oxidase>

Pyridoxine oxidase is an enzyme also called pyridoxine-4-oxidase. The pyridoxine oxidase may be an enzyme represented by the enzyme number EC1.1.3.12. Pyridoxine oxidase is an enzyme having an enzymatic activity that catalyzes a conversion reaction to pyridoxal by oxidizing pyridoxine with oxygen. Although pyridoxal or pyridoxine may also exist as a salt depending on a surrounding environment; however, in the disclosure, a description of enzymatic activity is given with the omission of the mention of a salt in order to simplify an expression. Pyridoxine oxidase consumes oxygen to produce hydrogen peroxide when oxidizing pyridoxine. Due to production of hydrogen peroxide which is harmful to organisms, it has been considered that it is possible to achieve a high yield when pyridoxine oxidase is used to produce another substance from pyridoxine. However, when the recombinant microorganism according to the disclosure is used, an unexpected effect of achieving the production of pyridoxamine or a salt thereof from pyridoxine or a salt thereof as a raw material at high production efficiency can be obtained.

The pyridoxine oxidase of EC1.1.3.12 may be a pyridoxine oxidase derived from, for example, Enterobacter cloacae, Mesorhizobium loti, Microbacterium luteolum, Ochrobactrum anthropi, Pseudomonas sp. MA-1, or the like. The pyridoxine oxidase from Microbacterium luteolum has the amino acid sequence of SEQ ID NO:1.

### <Pyridoxamine Synthetase>

The pyridoxamine synthetase used in the disclosure refers to any enzyme having an enzymatic activity of synthesizing pyridoxamine from pyridoxal. Pyridoxal or pyridoxamine may also exist as a salt depending on a surrounding environment; however, in the disclosure, a description of enzymatic activity is given with the omission of the mention of a salt in order to simplify an expression. Examples of the pyridoxamine synthetase include, for example, a pyridoxamine-pyruvate transaminase represented by the enzyme number EC 2.6.1.30, a pyridoxamine-oxaloacetate transaminase represented by EC 2.6.1.31, an aspartate transaminase represented by EC 2.6.1.1, and a pyridoxamine phosphate transaminase represented by EC 2.6.1.54.

The aspartate transaminase represented by EC 2.6.1.1 is a holoenzyme using pyridoxal phosphate as a coenzyme, and has an enzymatic activity of produce glutamic acid and oxaloacetic acid by transferring the amino group of aspartic acid to 2-oxoglutaric acid. The aspartate transaminase in a state of an apoenzyme not bound to pyridoxal phosphate is known synthesize pyridoxamine by transferring the amino group of glutamic acid or aspartic acid to pyridoxal (Journal of Biological Chemistry, January 1962, Vol. 237, No. 1, p. 127-132). In other words, an apoenzyme form of the aspartate transaminase represented by EC 2.6.1.1 is the pyridoxamine-oxaloacetate transaminase of EC 2.6.1.31. For this reason, aspartate transaminase exists as an apoenzyme when a coenzyme pyridoxal phosphate is absent or present in a small amount, and synthesizes pyridoxamine.

The pyridoxamine synthetase has an enzymatic activity of producing pyridoxamine or a salt thereof by oxidizing an amino group moiety of a specific amino acid to (═O) and transferring the amino group in the synthesis of pyridoxamine or a salt thereof from pyridoxal or a salt thereof. For example, the pyridoxamine-pyruvate transaminase can use both L-alanine and D-alanine, each of the pyridoxamine-oxaloacetate transaminase and the aspartate transaminase in an apoenzyme state can use D-aspartic acid, L-aspartic acid, D-glutamic acid, and L-glutamic acid, and the pyridoxamine phosphate transaminase can use D-glutamic acid.

The pyridoxamine-pyruvate transaminase may be derived from, for example, a microorganism belonging to the phylum Proteobacteria, a microorganism belonging to the phylum Actinobacteria, a microorganism belonging to the phylum Spirocheta, or a microorganism belonging to the phylum Filmictes. The pyridoxamine-pyruvate transaminase may be a pyridoxamine-pyruvate transaminase derived from, for example, Mesorhizobium loti, Ochrobactrum anthropi, or the genus Pseudomonas (such as Pseudomonas sp. MA-1). Here, the pyridoxamine-pyruvate transaminase from Mesorhizobium loti has the amino acid sequence of SEQ ID NO:2, for example.

The pyridoxamine-pyruvate transaminase may also be, for example, a pyridoxamine-pyruvate transaminase (having the amino acid sequence of SEQ ID NO:18) derived from Mesorhizobium sp. YR577, a pyridoxamine-pyruvate transaminase (having the amino acid sequence of SEQ ID NO:19) derived from Pseudaminobacter salicylatoxidans, a pyridoxamine-pyruvate transaminase (having the amino acid sequence of SEQ ID NO:20) derived from Bauldia litoralis, a pyridoxamine-pyruvate transaminase (having the amino acid sequence of SEQ ID NO:21) derived from Skermanella stibiiresistens, a pyridoxamine-pyruvate transaminase (having the amino acid sequence of SEQ ID NO:22) derived from Rhizobium sp. AC44/96, a pyridoxamine-pyruvate transaminase (having the amino acid sequence of SEQ ID NO:23) derived from Erwinia toletana, or a pyridoxamine-pyruvate transaminase (having the amino acid sequence of SEQ ID NO:24) derived from Herbiconiux ginsengi.

The pyridoxamine-oxaloacetate transaminase may be, for example, a pyridoxamine-oxaloacetate transaminase derived from Escherichia coli, Oryctolagus cuniculus, or Rattus norvegicus. For example, the pyridoxamine-oxaloacetate transaminase from Escherichia coli has the amino acid sequence of SEQ ID NO:4. The aspartate transaminase may be an aspartate transaminase derived from, for example, Escherichia coli, Trichoderma viride, or the like. The pyridoxamine phosphate transaminase may be a pyridoxamine phosphate transaminase derived from, for example, Clostridium butyricum.

<Hydrogen Peroxide-Degrading Enzyme>

The recombinant microorganism according to the disclosure may further include a gene encoding a hydrogen peroxide-degrading enzyme having an enzymatic activity of degrading hydrogen peroxide. The hydrogen peroxide-degrading enzyme used in the disclosure refers to any enzyme having an enzymatic activity of degrading hydrogen peroxide generated when the pyridoxine oxidase oxidizes pyridoxine. In the case of further including the gene encoding the hydrogen peroxide-degrading enzyme, accumulation of hydrogen peroxide that is harmful to organisms and enzymatic activity can be further reduced, which is advantageous in terms of further improving the production efficiency of pyridoxamine or a salt thereof and prolonging the duration of a reaction. The hydrogen peroxide-degrading enzyme may have an enzymatic activity of regenerating oxygen. The presence of the enzymatic activity of regenerating oxygen can increase the concentration of oxygen in a reaction system, particularly when producing pyridoxamine or a salt thereof in a low oxygen environment, and is advantageous from the viewpoint of further improving the production efficiency of pyridoxamine or a salt thereof.

Examples of such hydrogen peroxide-degrading enzymes include an enzyme represented by the enzyme number (EC) 1.11.1.1, 1.11.1.2, 1.11.1.3, 1.11.1.5, 1.11.1.6, 1.11.1.7, 1.11.1.8, 1.11.1.9, 1.11.1.10, 1.11.1.11, 1.11.1.13, 1.11.1.14, 1.11.1.16, 1.11.1.17, 1.11.1.18, 1.11.1.19, 1.11.1.21, or 1.11.1.23. Among them, a catalase represented by the enzyme number EC 1.11.1.6 and a catalase peroxidase represented by EC 1.11.1.21 are preferable from the viewpoint of the oxygen regenerating ability, and a catalase represented by the enzyme number EC 1.11.1.6 is more preferable.

The hydrogen peroxide-degrading enzyme may be a catalase derived from, for example, Listeria seeligeri, Escherichia coli) or Saccharomyces cerevisiae. Alternatively, the hydrogen peroxide-degrading enzyme may be a catalase peroxidase derived from, for example, Escherichia coli. For example, the catalase from Listeria seeligeri has the amino acid sequence of SEQ ID NO:3.

Each of the pyridoxine oxidase, the pyridoxamine synthetase, and the hydrogen peroxide-degrading enzyme may be a protein that has a known amino acid sequence (such as an amino acid sequence encoded by a gene naturally occurring in an organism, or an amino acid sequence encoded by a gene possessed by a naturally occurring microorganism such as the above-described microorganism) having the enzymatic activity and that is unmodified, or may be a protein that has an amino acid sequence obtained by modifying such an amino acid sequence as long as the activity of the enzymatic activity (such as the above-described enzymatic activity) is not lost. Examples of the modification include insertion, deletion, substitution of an amino acid residue, and addition of an additional amino acid residue to either or both of the N-terminus and the C-terminus of an amino acid sequence. In a case in which there is one or more of the insertion, deletion, and substitution of the amino acid residue, the number of each of insertion, deletion, and substitution, if present, may be, for example, from 1 to 30 amino acid residues, from 1 to 20 amino acid residues, from 1 to 10 amino acid residues, or from 1 to 5 amino acid residues; and the total number of insertion, deletion, and substitution of the amino acid residue may be, for example, from 1 to 50 amino acid residues, from 1 to 30 amino acid residues, from 1 to 10 amino acid residues, or from 1 to 5 amino acid residues. The number of amino acid residue added to the terminus, if present, may be, for example, from 1 to 50 amino acid residues, from 1 to 30 amino acid residues, from 1 to 10 amino acid residues, or from 1 to 5 amino acid residues per one terminus. The additional amino acid residue may form a signal sequence for extracellular secretion or the like. Examples of the signal sequence include an E. coli OmpA signal sequence.

Alternatively, each of the enzymes may be a protein having a known amino acid sequence (such as an amino acid sequence encoded by a gene naturally occurring in an organism) having the enzymatic activity per se, or a protein having an amino acid sequence that has 80% or more, 85% or more, 90% or more, or 95% or more sequence identity with a known amino acid sequence (such as an amino acid sequence encoded by a gene naturally occurring in an organism) having the enzymatic activity and that has a desired enzymatic activity (such as the above-mentioned enzymatic activity). Here, the sequence identity can be evaluated by using, for example, a BLAST (registered trademark, National Library of Medicine) program with default parameters.

For example, the pyridoxine oxidase may be, for example, a protein having the amino acid sequence of SEQ ID NO:1, or may be a protein having an amino acid sequence in which one or more of the substitution, deletion, or the insertion of an amino acid residue, or addition of an additional amino acid residue to either or both of the N-terminus and C-terminus of the amino acid sequence are performed in the amino acid sequence of SEQ ID NO:1. Examples of the degree of the substitution, deletion, and insertion of an amino acid residue, and the addition of an additional amino acid residue to either or both of the N-terminus and the C-terminus of the amino acid sequence are as described above.

Alternatively, the pyridoxine oxidase may be a protein having the amino acid sequence of SEQ ID NO:1, or may be a protein having an amino acid sequence having, for example, 80% or more, 85% or more, 90% or more, or 95% or more sequence identity with the amino acid sequence of SEQ ID NO:1.

In a case of using such a protein having an amino acid sequence similar to the amino acid sequence of SEQ ID NO:1 as described above, it is necessary that the protein has an activity for a pyridoxine oxidase. The pyridoxine oxidase activity can be measured by, for example, adding a protein to be tested to an aqueous solution including pyridoxine as a substrate in the presence of oxygen, and quantifying the amount of produced pyridoxal by high performance liquid chromatography, or forming a Schiff base between produced pyridoxal and an amine such as trishydroxymethylaminomethane and quantifying the amount of the Schiff base with absorbance measurement at 415 nm or the like.

Alternatively, the pyridoxamine synthetase may be a protein having the amino acid sequence of any one of SEQ ID NO:2 or SEQ ID NO:18 to SEQ ID NO:24, or a protein having an amino acid sequence having, for example, 80% or more, 85% or more, 90% or more, or 95% or more sequence identity with at least one of the amino acid sequence of SEQ ID NO:2, the amino acid sequence of SEQ ID NO:18, the amino acid sequence of SEQ ID NO:19, the amino acid sequence of SEQ ID NO:20, the amino acid sequence of SEQ ID NO:21, the amino acid sequence of SEQ ID NO:22, the amino acid sequence of SEQ ID NO:23, or the amino acid sequence of SEQ ID NO:24. It is necessary that the protein has an activity for a pyridoxamine synthetase. In this case, the enzymatic activity of synthesizing pyridoxamine from pyridoxal can be measured by, for example, adding a protein to be tested to an aqueous solution including pyridoxal as a substrate and L-alanine, and quantifying the amount of produced pyridoxamine by high performance liquid chromatography or the like.

Alternatively, the pyridoxamine synthetase may be a pyridoxamine synthetase including at least one of the following partial amino acid sequence (c), partial amino acid sequence (d), partial amino acid sequence (e), partial amino acid sequence (f), partial amino acid sequence (g), or partial amino acid sequence (h), and having an enzymatic activity of synthesizing pyridoxamine from pyridoxal. In this case, the enzymatic activity of synthesizing pyridoxamine from pyridoxal can be measured by, for example, adding a protein to be tested to an aqueous solution including pyridoxal and L-alanine as substrates, and quantifying the amount of produced pyridoxamine by high performance liquid chromatography or the like.

(c) X₈X₉X₁₀X₁₁X₁₂X₁₃ (SEQ ID NO:39)

wherein X₈ represents L, M, I or V,

X₉ represents H or Q,

X₁₀ represents C or A,

X₁₁ represents E or D,

X₁₂ represents P or A, and

X₁₃ represents V, I, L or A;

(d) X₁₄X₁₅TPSGTX₁₆X₁₇ (SEQ ID NO:40)

wherein X₁₄ represents H or S,

X₁₅ represents D or E,

X₁₆ represents I, V, or L, and

X₁₇ represents N or T;

(e) X₁₅DX₁₉VSX₂₀X₂₁ (SEQ ID NO:41)

wherein X₁₅ represents V, I, or A,

X₁₉ represents A, T, or S,

X₂₀ represents S, A, or G, and

X₂₁ represents F, W, or V;

(f) X₂₂X₂₃X₂₄KCX₂₅GX₂₆X₂₇P (SEQ ID NO:42)

wherein X₂₂ represents G or S,

X₂₃ represents P, S, or A,

X₂₄ represents N, S, A, or Q,

X₂₅ represents L or M,

X₂₆ represents A, S, C, or G, and

X₂₇ represents P, T, S, or A;

(g) X₂₈X₂₉X₃₀X₃₁SX₃₂GX₃₃X₃₄ (SEQ ID NO:43)

wherein X₂₈ represents G or D,

X₂₉ represents V or I,

X₃₀ represents V, T, A, S, M, I, or L,

X₃₁ represents F, M, L, I, or V,

X₃₂ represents S, A, T, I, L, or H,

X₃₃ represents R, M, or Q, and

X₃₄ represents R, A, D, H, or K; and

(h) X₃₅X₃₆RX₃₇X₃₈HMGX₃₉X₄₀A (SEQ ID NO:44)

wherein X₃₅ represents L or V,

X₃₆ represents T, I, V, or L,

X₃₇ represents I, V, or L,

X₃₈ represents G or S,

X₃₉ represents P, A, or R, and

X₄₀ represents T, V, or S.

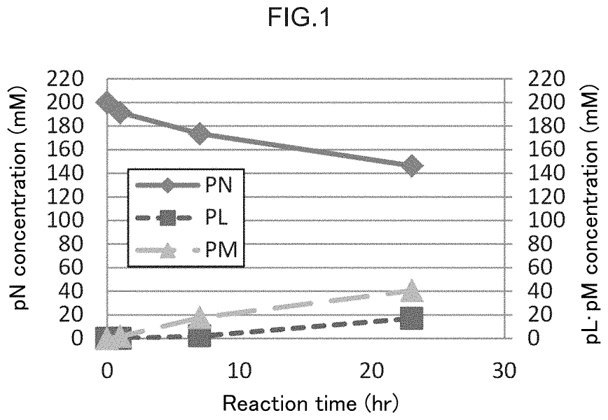

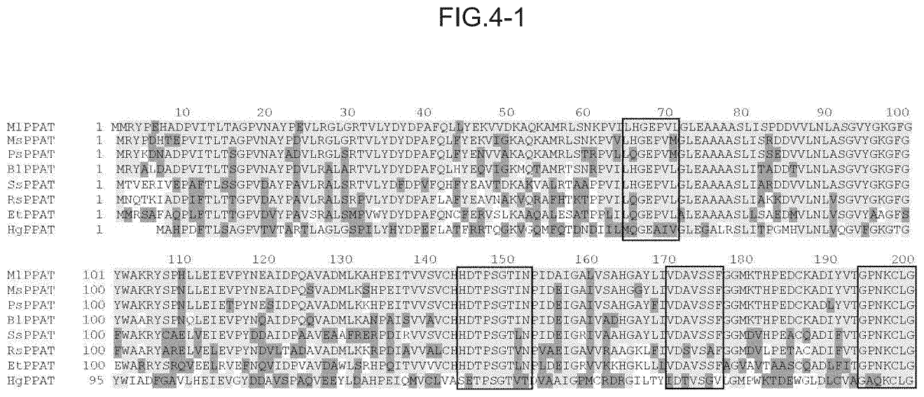

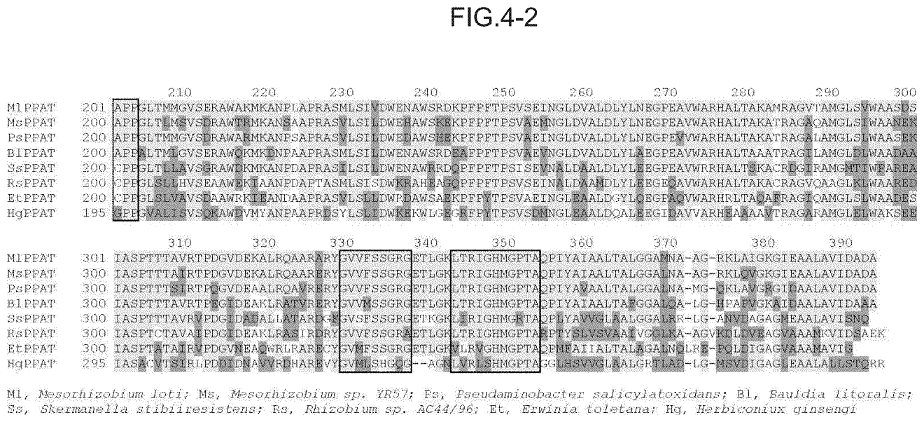

FIG. 4-1 and FIG. 4-2 illustrate alignments of the sequences of SEQ ID NO:2 and SEQ ID NO:18 to SEQ ID NO:24, respectively. In FIG. 4-1 and FIG. 4-2, MIPPAT represents a pyridoxamine-pyruvate transaminase from Mesorhizobium loti, MsPPAT represents a pyridoxamine-pyruvate transaminase from Mesorhizobium sp. YR577, PsPPAT represents a pyridoxamine-pyruvate transaminase from Pseudaminobacter salicylatoxidans, B₁PPAT represents a pyridoxamine-pyruvate transaminase from Bauldia litoralis, SsPPAT represents a pyridoxamine-pyruvate transaminase from Skermanella stibiiresistens, RsPPAT represents a pyridoxamine-pyruvate transaminase from Rhizobium sp. AC44/96, EtPPAT represents a pyridoxamine-pyruvate transaminase from Erwinia toletana, and HgPPAT represents a pyridoxamine-pyruvate transaminase from Herbiconiux ginsengi. The partial amino acid sequence (c) corresponds to amino acid residues corresponding to the 65th to 70th amino acid residues from the N-terminus of the pyridoxamine-pyruvate transaminase from Mesorhizobium loti in alignment, the partial amino acid sequence (d) corresponds to amino acid residues corresponding to the 144th to 152nd amino acid residues from the N-terminus of the pyridoxamine-pyruvate transaminase from Mesorhizobium loti in alignment, the partial amino acid sequence (e) corresponds to amino acid residues corresponding to the 170th to 176th amino acid residues from the N-terminus of the pyridoxamine-pyruvate transaminase from Mesorhizobium loti in alignment, the partial amino acid sequence (f) corresponds to amino acid residues corresponding to the 194th to 203rd amino acid residues from the N-terminus of the pyridoxamine-pyruvate transaminase from Mesorhizobium loti in alignment, the partial amino acid sequence (g) corresponds to the amino acid residues corresponding to the 329th to 337th amino acid residues from the N-terminus of the pyridoxamine-pyruvate transaminase from Mesorhizobium loti in alignment, and the partial amino acid sequence (h) corresponds to the amino acid residues corresponding to the 343rd to 353rd amino acid residues from the N-terminus of pyridoxamine-pyruvate transaminase from Mesorhizobium loti in alignment. The partial amino acid sequence (c) preferably exists in the region of the 55th to 80th amino acid residues from the N-terminus of the protein, and more preferably exists in the region of the 56th to 75th amino acid residues from the N-terminus. The partial amino acid sequence (d) preferably exists in the region of the 134th to 162nd amino acid residues from the N-terminus of a protein, and more preferably exists in the region of the 139th to 157th amino acid residues from the N-terminus. The partial amino acid sequence (e) preferably exists in the region of the 160th to 186th amino acid residues from the N-terminus of a protein, and more preferably exists in the region of the 165th to 181st amino acid residues from the N-terminus. The partial amino acid sequence (f) preferably exists in the region of the 184th to 213rd amino acid residues from the N-terminus of a protein, and more preferably exists in the region of the 189th to 208th amino acid residues from the N-terminus. The partial amino acid sequence (g) preferably exists in the region of the 319th to 347th amino acid residues from the N-terminus of a protein, and more preferably exists in the region of the 324th to 342nd amino acid residues from the N-terminus. The partial amino acid sequence (h) preferably exists in the region of the 333rd to 363rd amino acid residues from the N-terminus of a protein, and more preferably exists in the region of the 338th to 358th amino acid residues from the N-terminus. In the disclosure, alignment between sequences can be performed by using, for example, a BLAST (registered trademark, National Library of Medicine) program with default parameters.

In the disclosure, “amino acid residue corresponding to the Xth amino acid residue from N-terminus of enzyme A” in the amino acid sequence of enzyme B refers to an amino acid residue on the amino acid sequence of the enzyme B, corresponding to the Xth amino acid residue from the N-terminus of the amino acid sequence of enzyme A in a case in which the amino acid sequence of enzyme A is aligned with the amino acid sequence of the enzyme B.

As can be seen from FIG. 4-1 and FIG. 4-2, the partial amino acid sequence (c), the partial amino acid sequence (d), the partial amino acid sequence (e), the partial amino acid sequence (f), the partial amino acid sequence (g), and the partial amino acid sequence (h) are regions highly conserved in a group of pyridoxamine-pyruvate transaminases. Therefore, a variation of the pyridoxamine-pyruvate transaminases including at least one of the partial amino acid sequence (c), the partial amino acid sequence (d), the partial amino acid sequence (e), the partial amino acid sequence (f), the partial amino acid sequence (g), or the partial amino acid sequence (h) is considered to be highly probable to achieve functioning as the pyridoxamine synthetase in the disclosure. In the pyridoxamine-pyruvate transaminase from Mesorhizobium loti, an amino acid residue corresponding to the 197th lysine residue from the N-terminus on the alignment is important for binding to pyridoxal, an amino acid residue corresponding to the 68th glutamate residue from the N-terminus on the alignment is important for catalytic activity, an amino acid residue corresponding to the 171st aspartate residue from the N-terminus on the alignment and an amino acid residue corresponding to the 146th threonine residue from the N-terminus on the alignment assist binding to pyridoxal, and an amino acid residue corresponding to the 336th arginine residue from the N-terminus on the alignment and an amino acid residue corresponding to the 345th arginine residue from the N-terminus on the alignment are considered to be important for recognizing an amino acid (Journal of Biological Chemistry, 2008, vol. 283, No. 2, pp 1120-1127), and the amino acid residues are important residues in view of the function of the pyridoxamine synthetase. In a case in which these residues are conserved, it is considered to be highly probable to achieve functioning as the pyridoxamine synthetase in the disclosure. However, an amino acid residue corresponding to the 68th glutamate residue from the N-terminus of the pyridoxamine-pyruvate transaminase from Mesorhizobium loti on the alignment may be not only glutamate but also aspartate. An amino acid residue corresponding to the 336th arginine residue from the N-terminus of the pyridoxamine-pyruvate transaminase from Mesorhizobium loti on the alignment may be not only arginine but also methionine or glutamine.

Examples of other expressions of a region including the amino acid residue corresponding to the 68th glutamate residue from the N-terminus of the pyridoxamine-pyruvate transaminase from Mesorhizobium loti on the alignment include the following partial amino acid sequence (c-1). Instead of the partial amino acid sequence (c), the pyridoxamine synthetase may include the partial amino acid sequence (c-1).

(c-1) X₁X₂X₃X₄X₅X₆X₇X₈X₉X₁₀X₁₁X₁₂X₁₃X₁₄X₁₅X₁₆X₁₇X₁₈X₁₉ (SEQ ID NO:45)

wherein X₁ represents V, L, I, or M,

X₂ represents I, L, or V,

X₃ represents L, M, I, or V,

X₄ represents H or Q,

X₅ represents C, or A,

X₆ represents E or D,

X₇ represents P or A,

X₈ represents V, I, A, or L,

X₉ represents L, M, P, or V,

X₁₀ represents G or A,

X₁₁ represents L or I,

X₁₂ represents E or Q,

X₁₃ represents A or

X₁₄ represents A or V,

X₁₅ represents A or L,

X₁₆ represents A, L, H, or Y,

X₁₇ represents S, or A,

X₁₈ represents L, F, V, or A, and

X₁₉ represents I, F, V, or L.

The partial amino acid sequence (c-1) corresponds to amino acid residues corresponding to the 63rd to 81st amino acid residues from the N-terminus of the pyridoxamine-pyruvate transaminase from Mesorhizobium loti on the alignment. The partial amino acid sequence (c-1) preferably exists in the region of the 53rd to 91st amino acid residues from the N-terminus of a protein, and more preferably exists in the region of the 58th to 86th amino acid residues from the N-terminus.

Examples of other expressions of a region including the amino acid residue corresponding to the 146th threonine residue from the N-terminus of a pyridoxamine-pyruvate transaminase from Mesorhizobium loti on the alignment include the following partial amino acid sequence (d-1). Instead of the partial amino acid sequence (d), the pyridoxamine synthetase may include the partial amino acid sequence (d-1).

(d-1) X₁X₂X₃X₄X₅X₆X₇X₈TPSGTX₉X₁₀X₁₁X₁₂X₁₃X₁₄X₁₅X₁₆ (SEQ ID NO:46)

wherein X₁ represents V, I, L, or M,

X₂ represents V or I,

X₃ represents S, A, V, C, or F,

X₄ represents V, I, A, L, or T,

X₅ represents C or V,

X₆ represents H, N, or A,

X₇ represents H or S,

X₈ represents D or E,

X₉ represents I, V, or L,

X₁₀ represents N or T,

X₁₁ represents P or D,

X₁₂ represents I, V, L, or A,

X₁₃ represents D, N, E, A, Q, V, R, or P,

X₁₄ represents A, E, Q, or D,

X₁₅ represents I or L, and

X₁₆ represents G or A.

The partial amino acid sequence (d-1) corresponds to the amino acid residues corresponding to the 138th to 158th amino acid residues from the N-terminus of the pyridoxamine-pyruvate transaminase from Mesorhizobium loti on the alignment. The partial amino acid sequence (d-1) preferably exists in the region of the 128th to 168th amino acid residues from the N-terminus of a protein, and more preferably exists in the region of the 133rd to 163rd amino acid residues from the N-terminus.

Examples of other expressions of a region including the amino acid residue corresponding to the 171st aspartate residue from the N-terminus of a pyridoxamine-pyruvate transaminase from Mesorhizobium loti include the following partial amino acid sequence (e-1). Instead of the partial amino acid sequence (e), the pyridoxamine synthetase may include the partial amino acid sequence (e-1).

(e-1) X₁X₂X₃X₄X₅X₆DX₇VSX₈X₉X₁₀X₁₁X₁₂ (SEQ ID NO:47)

in which X₁ represents D, or A,

X₂ represents A, K, T, Q, R, or E,

X₃ represents Y, N, L, or F,

X₄ represents L, F, M, or V,

X₅ represents I, L, or Y,

X₆ represents V, A, or I,

X₇ represents A, S, or T,

X₈ represents S, A, or

X₉ represents F, W, or V,

X₁₀ represents A, or L,

X₁₁ represents G or S, and

X₁₂ represents M, V, or L.

The partial amino acid sequence (e-1) corresponds to the amino acid residues corresponding to the 165th to 179th amino acid residues from the N-terminus of the pyridoxamine-pyruvate transaminase from Mesorhizobium loti on the alignment. The partial amino acid sequence (e-1) preferably exists in the region of the 155th to 189th amino acid residues from the N-terminus of the protein, and more preferably exists in the region of the 160th to 184th amino acid residues from the N-terminus.

Examples of other expressions of a region including the amino acid residue corresponding to the 197th lysine residue from the N-terminus of a pyridoxamine-pyruvate transaminase from Mesorhizobium loti on the alignment include the following partial amino acid sequence (f-1). Instead of the partial amino acid sequence (f), the pyridoxamine synthetase may include the partial amino acid sequence (f-1).

(f-1) X₁X₂X₃X₄X₅X₆X₇X₈X₉KCX₁₀GX₁₁X₁₂PX₁₃X₁₄X₁₅X₁₆X₁₇X₁₈X₁₉S (SEQ ID NO:48)

wherein X₁ represents A, S, V, or I,

X₂ represents D, or A,

X₃ represents I, L, F, V, or M,

X₄ represents Y, F, L, or C,

X₅ represents V or I,

X₆ represents T or A,

X₇ represents G or S,

X₈ represents P, S, or A,

X₉ represents N, S, Q, or A,

X₁₀ represents L or M,

X₁₁ represents A, S, C, or

X₁₂ represents P, T, S, or A,

X₁₃ represents A, or S,

X₁₄ represents L or V,

X₁₅ represents T, S, or A,

X₁₆ represents M, I, L, V, or F,

X₁₇ represents M, L, V, A, or I,

X₁₅ represents A, H, or S, and

X₁₉ represents V, I, or A.

The partial amino acid sequence (f-1) corresponds to the amino acid residues corresponding to the 188th to 211st amino acid residues from the N-terminus of the pyridoxamine-pyruvate transaminase from Mesorhizobium loti on the alignment. The partial amino acid sequence (f-1) preferably exists in the region of from the 178th to 221st amino acid residues from the N-terminus of the protein, and more preferably exists in the region of from the 183rd to 216th amino acid residues from the N-terminus.

Examples of other expressions of a region including the amino acid residue corresponding to the 336th arginine residue from the N-terminus of the pyridoxamine-pyruvate transaminase from Mesorhizobium loti on the alignment include the following partial amino acid sequence (g-1). Instead of the partial amino acid sequence (g), the pyridoxamine synthetase may include the partial amino acid sequence (g-1).

(g-1) X₁X₂X₃X₄X₅SX₆GX₇X₈ (SEQ ID NO:49)

wherein X₁ represents Y, F, H, or S,

X₂ represents G or D,

X₃ represents V or I,

X₄ represents V, T, A, S, M, I, or L,

X₅ represents F, M, L, I, or V,

X₆ represents S, A, T, I, L, or H,

X₇ represents R, M, or Q, and

X₈ represents R, A, D, H, or K.

The partial amino acid sequence (g-1) corresponds to the amino acid residues corresponding to the 328th to 337th amino acid residues from the N-terminus of the pyridoxamine-pyruvate transaminase from Mesorhizobium loti on the alignment. The partial amino acid sequence (g-1) preferably exists in the region of the 318th to 347th amino acid residues from the N-terminus of the protein, and more preferably exists in the region of the 323rd to 342nd amino acid residues from the N-terminus.

Examples of other expressions of a region including the amino acid residue corresponding to the 345th arginine residue from the N-terminus of the pyridoxamine-pyruvate transaminase from Mesorhizobium loti on the alignment include the following partial amino acid sequence (h-1). Instead of the partial amino acid sequence (h), the pyridoxamine synthetase may include the partial amino acid sequence (h-1).

(h-1) X₁X₂X₃X₄X₅RX₆X₇HMGX₈X₉AX₁₀X₁₁ (SEQ ID NO:50)

wherein X₁ represents L, Q, K, A, F, Y, or W,

X₂ represents N, H, or D,

X₃ represents K, R, or N,

X₄ represents L or V,

X₅ represents T, I, V, or L,

X₆ represents I, V, or L,

X₇ represents G or S,

X₈ represents P, A, or R,

X₉ represents T, V, or S,

X₁₀ represents Q, R, E, K, H, Y, or G, and

X₁₁ represents P or G

The partial amino acid sequence (h-1) corresponds to the amino acid residues corresponding to the 340th to 355th amino acid residues from the N-terminus of the pyridoxamine-pyruvate transaminase from Mesorhizobium loti on the alignment. The partial amino acid sequence (h-1) preferably exists in the region of the 330th to 365th amino acid residues from the N-terminus of a protein, and more preferably exists in the region of the 335th to 360th amino acid residues from the N-terminus.

The pyridoxamine synthetase may be, for example, a protein having the amino acid sequence of SEQ ID NO:2, or may be a protein having an amino acid sequence in which one or more of the substitution, deletion, or insertion of an amino acid residue, or the addition of an additional amino acid residue to either or both of the N-terminus and C-terminus of the amino acid sequence are performed in the amino acid sequence of SEQ ID NO:2. Examples of the degrees of the substitution, deletion, and insertion of the amino acid residue, and the addition of an additional amino acid residue to either or both of the N-terminus and the C-terminus of the amino acid sequence are as described above.

Alternatively, the pyridoxamine synthetase may be a protein having the amino acid sequence of SEQ ID NO:2, or may be a protein having an amino acid sequence having, for example, 80% or more, 85% or more, 90% or more, or 95% or more sequence identity with the amino acid sequence of SEQ ID NO:2.

In the case of using such a protein having an amino acid sequence similar to the amino acid sequence of SEQ ID NO:2 as described above, it is necessary that the protein has an activity for pyridoxamine synthetase (an enzymatic activity of synthesizing pyridoxamine from pyridoxal, also referred to as pyridoxamine synthetase activity in the disclosure). The enzymatic activity (pyridoxamine synthetase activity) of synthesizing pyridoxamine from pyridoxal can be measured by, for example, adding a protein to be tested to an aqueous solution including pyridoxal as a substrate, and necessary amino acid (for example, L-alanine in the case of a protein having an amino acid sequence similar to the amino acid sequence of SEQ ID NO:2), and quantifying the amount of produced pyridoxamine by high performance liquid chromatography or the like.

Alternatively, the pyridoxamine synthetase may be, for example, a protein having an amino acid sequence of SEQ ID NO:4, or may be a protein having an amino acid sequence in which one or more of the substitution, deletion, or the insertion of an amino acid residue, or the addition of an additional amino acid residue to either or both of the N-terminus and C-terminus are performed in the amino acid sequence of SEQ ID NO:4. Examples of the degrees of the substitution, deletion, and insertion of an amino acid residue, and the addition of an additional amino acid residue to either or both of the N-terminus and the C-terminus of the amino acid sequence are as described above.

Alternatively, the pyridoxamine synthetase may be a protein having the amino acid sequence of SEQ ID NO:4, or may be a protein having an amino acid sequence having, for example, 80% or more, 85% or more, 90% or more, or 95% or more sequence identity with the amino acid sequence of SEQ ID NO:4.

In a case of using such a protein having an amino acid sequence similar to the amino acid sequence of SEQ ID NO:4 as described above, it is necessary that the protein has an activity for a pyridoxamine synthetase (an enzymatic activity of synthesizing pyridoxamine from pyridoxal, also referred to as pyridoxamine synthetase activity in the disclosure). The enzyme (pyridoxamine synthetase activity) of synthesizing pyridoxamine from pyridoxal can be measured by, for example, adding a protein to be tested to an aqueous solution including pyridoxal as a substrate, and necessary amino acid (for example, L-glutamic acid, L-aspartic acid, or a salt thereof in the case of a protein having an amino acid sequence similar to the amino acid sequence of SEQ ID NO:4), and quantifying the amount of produced pyridoxamine by high performance liquid chromatography or the like.

The hydrogen peroxide-degrading enzyme may be, for example, a protein having the amino acid sequence of SEQ ID NO:3, or may be a protein having an amino acid sequence in which one or more of the substitution, deletion, or the insertion of an amino acid residue, or addition of an additional amino acid residue to either or both of the N-terminus and C-terminus of the amino acid sequence are performed in the amino acid sequence of SEQ ID NO:3. Examples of the degrees of the substitution, deletion, and insertion of the amino acid residue, and the addition of an additional amino acid residue to either or both of the N-terminus and the C-terminus of the amino acid sequence are as described above.

Alternatively, the hydrogen peroxide-degrading enzyme may be a protein having the amino acid sequence of SEQ ID NO:3, or may be a protein having an amino acid sequence having, for example, 80% or more, 85% or more, 90% or more, or 95% or more sequence identity with the amino acid sequence of SEQ ID NO:3.

In a case of using such a protein having an amino acid sequence similar to the amino acid sequence of SEQ ID NO:3 as described above, it is necessary that the protein has an activity for a hydrogen peroxide-degrading enzyme. The activity of the hydrogen peroxide-degrading enzyme can be measured by, for example, adding a protein to be tested to an aqueous solution of hydrogen peroxide as a substrate and quantifying decrease in the amount of hydrogen peroxide using decrease in absorbance at 240 nm or the like.

<Gene Encoding Pyridoxine Oxidase, Gene Encoding Pyridoxamine Synthetase, and Gene Encoding Hydrogen Peroxide-Degrading Enzyme>

The gene encoding the pyridoxine oxidase may be any gene encoding the pyridoxine oxidase described above. The gene encoding the pyridoxamine synthetase may be any gene encoding the pyridoxamine synthetase described above. The gene encoding the hydrogen peroxide-degrading enzyme may be any gene encoding the hydrogen peroxide-degrading enzyme described above. Enzymes encoded by these genes are not limited to known amino acid sequences having the enzymatic having the enzymatic activities (for example, an amino acid sequence encoded by a gene naturally occurring in an organism), and may be enzymes having modified amino acid sequences different from the known amino acid sequences.

Such a gene may be a known gene such as a gene naturally possessed by the microorganism exemplified above as the microorganism (microorganism from which the enzyme is derived) having each enzyme, or a gene in which a nucleotide sequence is modified so that the nucleotide sequence encodes a modified amino acid sequence that is modified from the known amino acid sequence of the enzyme as described above as long as the desired enzymatic activity is obtained. Examples of the modified amino acid sequence include an amino acid sequence similar to any one of the amino acid sequences of SEQ ID NO:1 to SEQ ID NO:4 as described above. The nucleotide sequence of a gene encoding a particular amino acid sequence can be varied within degeneracy of a codon. In this case, it is preferable to use a codon frequently used in a microorganism used as a host of the recombinant microorganism from the viewpoint of gene expression efficiency.

It is also possible to design a nucleotide sequence of a gene based on an amino acid sequence to be encoded using the codon table. The designed nucleotide sequence may be obtained by modifying a known nucleotide sequence using genetic recombination technology, or may be obtained by chemically synthesizing the nucleotide sequence.

Examples of the method of modifying a nucleotide sequence include site-directed mutagenesis (Kramer, W. and frita, H. J., Methods in Enzymology, vol. 154, P.350 (1987)), recombinant PCR (PCR Technology, Stockton Press (1989)), a method of chemically synthesizing a specific part of DNA, a method of treating a gene with hydroxyamine, a method of treating a strain carrying a gene with ultraviolet irradiation or a chemical agent such as nitrosoguanidine or nitrous acid, and a method of using a commercially available mutagenesis kit.

For example, each of the gene encoding the pyridoxine oxidase, the gene encoding the pyridoxamine synthetase, and the gene encoding the hydrogen peroxide-degrading enzyme may be DNA having an unmodified nucleotide sequence that encodes a polynucleotide having the enzymatic activity (for example, a nucleotide sequence possessed by a gene naturally occurring in an organism such as the above-described microorganism), or DNA having a nucleotide sequence in which a modification is made in the above nucleotide sequence as long as the enzymatic activity (the above-described enzymatic activity) of an enzyme encoded by the nucleotide sequence is not lost. Examples of the modification include insertion, deletion, and substitution of a nucleotide, and addition of an additional nucleotide to either or both of the 5′ end and 3′ end of the nucleotide sequence. In a case in which there is one or more of the insertion, deletion, and substitution of the nucleotide, the number of each of the insertion, deletion, and substitution, if present, may be, for example, from 1 to 90 nucleotides, from 1 to 60 nucleotides, from 1 to 30 amino acid residues, from 1 to 20 amino acid residues, from 1 to 15 nucleotides, from 1 to 10 nucleotides, or from 1 to 5 nucleotides; and the total number of the insertion, deletion, and substitution of the nucleotide may be, for example from 1 to 100 nucleotides, from 1 to 50 nucleotides, from 1 to 30 nucleotides, from 1 to 10 nucleotides, or from 1 to 5 nucleotides. Insertion or deletion of a nucleotide may occur locally, and it is preferable that the entire nucleotide sequence does not have a large frame shift. The number of nucleotide added to the end if present, may be, for example, from 1 to 150 nucleotides, from 1 to 100 nucleotides, from 1 to 50 nucleotides, from 1 to 30 nucleotides, from 1 to 10 nucleotides, or from 1 to 5 nucleotides per end. Such an additional nucleotide may encode a signal sequence for extracellular secretion or the like.

Alternatively, the gene encoding each of the enzymes may be DNA having a known nucleotide sequence (for example, a nucleotide sequence of a gene naturally occurring in an organism) that encodes a polynucleotide having the enzymatic activity per se, or DNA that has a nucleotide sequence having 80% or more, 85% or more, 90% or more, or 95% or more sequence identity with the known nucleotide sequence and that encodes an enzyme having a desired enzymatic activity (such as the above-mentioned enzymatic activity). Here, the sequence identity can be evaluated by using, for example, a BLAST (registered trademark, National Library of Medicine) program with default parameters.

Alternatively, the gene encoding each of the enzymes may be DNA having a known nucleotide sequence (for example, a nucleotide sequence of a gene naturally occurring in an organism) that encodes a polynucleotide having the enzymatic activity per se, or DNA having a nucleotide sequence that hybridizes under a stringent condition with DNA having a nucleotide sequence complementary to the known nucleotide sequence, and that encodes an enzyme having a desired enzymatic activity (such as the above-described enzymatic activity). Hybridization under a stringent condition can be performed as follows.

The hybridization is performed on DNA of interest using DNA consisting of a nucleotide sequence complementary to a reference nucleotide sequence or a partial sequence thereof as a probe. After washing the resultant under a stringent condition, the significance of the hybridization of the probe to the nucleic acid of interest is confirmed. The length of the probe can be, for example, 20 or more continuous nucleotides, preferably 50 or more nucleotides, more preferably 100 or more nucleotides, and further preferably 200 or more nucleotides. It is also preferable to use DNA as a probe that has the same nucleotide length as the reference nucleotide sequence and that is complementary to the reference nucleotide over the entire length. Examples of the conditions for hybridization may include conditions commonly used by those skilled in the art for detecting a specific hybridization signal. Such conditions preferably mean stringent hybridization conditions and stringent wash conditions. Examples thereof include conditions that a temperature of 55° C. is maintained overnight, together with a probe, in a solution including 6×SSC (saline sodium citrate) (composition of 1×SSC: 0.15 M NaCl, 0.015 M sodium citrate, pH 7.0), 0.5% SDS, 5×Denhardt, and 100 mg/mL herring sperm DNA. Examples thereof may include subsequent washing of a filter in 0.2×SSC at 42° C. Examples of the stringent conditions may include conditions of 0.1×SSC and 50° C. in the step of washing a filter, and, in addition, examples of the stringent conditions can include 0.1×SSC and 65° C. in the same step.

For example, the gene encoding the pyridoxine oxidase may be, for example, DNA having the nucleotide sequence of SEQ ID NO:5 (the nucleotide sequence of a gene encoding a pyridoxine oxidase from Microbacterium luteolum), or DNA having a nucleotide sequence that hybridizes with DNA having a nucleotide sequence complementary to the nucleotide sequence of SEQ ID NO:5 under stringent conditions, and that encodes a protein having pyridoxine oxidase activity.

Alternatively, the gene encoding the pyridoxine oxidase may be, for example, DNA having the nucleotide sequence of SEQ ID NO:5, or DNA having a nucleotide sequence in which one or more of the substitution, deletion, or insertion of a nucleotide, or the addition of an additional nucleotide to either or both of the 5′ end and the 3′ end of the nucleotide sequence are performed in the nucleotide sequence of SEQ ID NO:5, and which encodes a protein having pyridoxine oxidase activity. Examples of the degrees of the substitution, deletion, and insertion of a nucleotide, and the addition of an additional nucleotide to either or both of the N-terminus and C-terminus of the nucleotide sequence are as described above.

Alternatively, the gene encoding the pyridoxine oxidase may be DNA having the nucleotide sequence of SEQ ID NO:5, or DNA having a nucleotide sequence that has, for example, 80% or more, 85% or more, 90% or more, or 95% or more sequence identity with the nucleotide sequence of SEQ ID NO:5, and that encodes a protein having pyridoxine oxidase activity.

The gene encoding the pyridoxamine synthetase may be, for example, DNA having the nucleotide sequence of SEQ ID NO:6 (the nucleotide sequence of a pyridoxamine-pyruvate transaminase gene from Mesorhizobium loti) or the nucleotide sequence of SEQ ID NO:8 (the nucleotide sequence of a pyridoxamine-oxaloacetate transaminase gene from E. coli), or may be DNA having a nucleotide sequence that hybridizes with DNA having a nucleotide sequence complementary to the nucleotide sequence of SEQ ID NO:6 or SEQ ID NO:8 under a stringent condition, and that encodes a protein having pyridoxamine synthetase activity.

Alternatively, the gene encoding the pyridoxamine synthetase may be, for example, DNA having the nucleotide sequence of SEQ ID NO:6 or SEQ ID NO: 8, or DNA having a nucleotide sequence in which one or more of the substitution, deletion, or insertion of a nucleotide, or the addition of an additional nucleotide to either or both of the 5′ end and the 3′ end of the nucleotide sequence are performed in the nucleotide sequence of SEQ ID NO:6 or SEQ ID NO:8, and which encodes a protein having pyridoxamine synthetase activity. Examples of the degrees of the substitution, deletion, and insertion of the nucleotide, and the addition of an additional nucleotide to either or both of the N-terminus and C-terminus of the nucleotide sequence are as described above.

Alternatively, the gene encoding the pyridoxamine synthetase may be DNA having the nucleotide sequence of SEQ ID NO:6 or SEQ ID NO:8, or may be DNA having a nucleotide sequence that has, for example, 80% or more, 85% or more, 90% or more, or 95% or more sequence identity with the nucleotide sequence of SEQ ID NO:6 or SEQ ID NO:8, and that encodes a protein having pyridoxamine synthetase activity.

Alternatively, the gene encoding the pyridoxamine synthetase may be, for example, DNA having the nucleotide sequence of any one of SEQ ID NO:6 or SEQ ID NO:25 to SEQ ID NO:31, or may be DNA having a nucleotide sequence that hybridizes with DNA having a nucleotide sequence complementary to the nucleotide sequence of any one of SEQ ID NO:6 or SEQ ID NO:25 to SEQ ID NO:31 under a stringent condition, and that encodes a protein having pyridoxamine synthetase activity.

Alternatively, the gene encoding the pyridoxamine synthetase may be, for example, DNA having the nucleotide sequence of any one of SEQ ID NO:6 or SEQ ID NO:25 to SEQ ID NO:31, or DNA having a nucleotide sequence in which one or more of the substitution, deletion, or insertion of a nucleotide, or the addition of an additional nucleotide to either or both of the 5′ end and the 3′ end of the nucleotide sequence are performed in the nucleotide sequence of any one of SEQ ID NO:6 or SEQ ID NO:25 to SEQ ID NO:31, and which encodes a protein having pyridoxamine synthetase activity. Examples of the degrees of the substitution, deletion, and insertion of a nucleotide, and the addition of an additional nucleotide either or both of the N-terminus and C-terminus of a nucleotide sequence are as described above.

Alternatively, the pyridoxamine synthetase may be DNA having the nucleotide sequence of any one of SEQ ID NO:6 or SEQ ID NO:25 to SEQ ID NO:31, or may DNA having a nucleotide sequence that has, for example, 80% or more, 85% or more, 90% or more, or 95% or more sequence identity with at least one of the nucleotide sequence of SEQ ID NO:6, the nucleotide sequence of SEQ ID NO:25 (the nucleotide sequence of a gene encoding pyridoxamine-pyruvate transaminase from Mesorhizobium sp. YR577), the nucleotide sequence of SEQ ID NO:26 (the nucleotide sequence of a gene encoding pyridoxamine-pyruvate transaminase from Pseudaminobacter salicylatoxidans), the nucleotide sequence of SEQ ID NO:27 (the nucleotide sequence of a gene encoding pyridoxamine-pyruvate transaminase from Bauldia litoralis), the nucleotide sequence of SEQ ID NO:28 (the nucleotide sequence of a gene encoding a pyridoxamine-pyruvate transaminase from Skermanella stibiiresistens), the nucleotide sequence of SEQ ID NO:29 (the nucleotide sequence of gene encoding a pyridoxamine-pyruvate transaminase from Rhizobium sp. AC44/96), the nucleotide sequence of SEQ ID NO:30 (the nucleotide sequence of a gene encoding a pyridoxamine-pyruvate transaminase from Erwinia toletana), or the nucleotide sequence of SEQ ID NO:31 (the nucleotide sequence of a gene encoding a pyridoxamine-pyruvate transaminase from Herbiconiux ginsengi), and that encodes a protein having pyridoxamine synthetase activity.

In the case of expression in a recombinant microorganism in which prokaryote such as E. coli is used as a host, a codon may be optimized to facilitate expression. For example, a nucleotide sequence may be modified so that a codon, which is most frequently used, of codons that encodes respective amino acids in a prokaryote as a host is highly frequently used as a codon for the amino acid. From such a viewpoint, as DNA including a gene encoding the pyridoxamine synthetase, for example, DNA having the region between the 18th nucleotide and the 3′ end of the nucleotide sequence of SEQ ID NO:10 or the region between the 18th nucleotide and the 3′ end of the nucleotide sequence of any one of SEQ ID NO:32 to SEQ ID NO:38 may be used, or DNA having a nucleotide sequence that hybridizes with DNA having a nucleotide sequence complementary to the region between 18th nucleotide and the 3′ end in the nucleotide sequence of SEQ ID NO:10 or the region between the 18th nucleotide and the 3′ end in the nucleotide sequence of any one of SEQ ID NO:32 to SEQ ID NO:38 under a stringent condition, and that encodes a protein having pyridoxamine synthetase activity may be used. Seventeen nucleotides from the 5′ end of the nucleotide sequence of SEQ ID NO:10 are included in an upstream region, and an initiation codon is included in the 18th nucleotide to the 20th nucleotide. Therefore, the region between the 18th nucleotide and the 3′ end of this nucleotide sequence may be used as a gene region encoding a pyridoxamine synthetase. Similarly, seventeen nucleotides from the 5′ end of each of the nucleotide sequences of SEQ ID NO:32 to SEQ ID NO:38 are included in an upstream, and an initiation codon is included in the 18th nucleotide to the 20th nucleotide. Therefore, the region between the 18th nucleotide and the 3′ end of these nucleotide sequences may be used as a gene region encoding a pyridoxamine synthetase.

Alternatively, the gene encoding the pyridoxamine synthetase may be, for example, DNA having the region between the 18th nucleotide and the 3′ end of the nucleotide sequence of SEQ ID NO:10 or the region between the 18th nucleotide and the 3′ end of the nucleotide sequence of any one of SEQ ID NO:32 to SEQ ID NO:38, or DNA having a nucleotide sequence in which one or more of the substitution, deletion, or insertion of a nucleotide, or the addition of an additional nucleotide to either or both of the 5′ end and the 3′ end of the are performed in the region between the 18th nucleotide and the 3′ end of the nucleotide sequence of the nucleotide sequence of SEQ ID NO:10 or the region between the 18th nucleotide and the 3′ end of the nucleotide sequence of any one of SEQ ID NO:32 to SEQ ID NO:38, and which encodes a protein having pyridoxamine synthetase activity. Examples of the degrees of the substitution, deletion, and insertion of a nucleotide, and the addition of an additional nucleotide to either or both of the N-terminus and C-terminus of the nucleotide sequence are as described above.

Alternatively, the pyridoxamine synthetase may be DNA having the region between the 18th nucleotide and the 3′ end of the nucleotide sequence of SEQ ID NO:10 or the region between the 18th nucleotide and the 3′ end of any one of nucleotide sequences of SEQ ID NO:32 to SEQ ID NO:38, or may be DNA having a nucleotide sequence that has, for example, 80% or more, 85% or more, 90% or more, or 95% or more sequence identity with at least one of the region between the 18th nucleotide and the 3′ end of the nucleotide sequence of SEQ ID NO:10 (codon-optimized nucleotide sequence of a gene encoding pyridoxamine-pyruvate transaminase from Mesorhizobium loti), the region between the 18th nucleotide and the 3′ end of the nucleotide sequence of SEQ ID NO:32 (codon-optimized nucleotide sequence of a gene encoding pyridoxamine-pyruvate transaminase from Mesorhizobium sp. YR577), the region between the 18th nucleotide and the 3′ end of the nucleotide sequence of SEQ ID NO:33 (codon-optimized nucleotide sequence of a gene encoding pyridoxamine-pyruvate transaminase from Pseudaminobacter salicylatoxidans), the region between the 18th nucleotide and the 3′ end of the nucleotide sequence of SEQ ID NO:34 (codon-optimized nucleotide sequence of a gene encoding pyridoxamine-pyruvate transaminase from Bauldia litoralis), the region between the 18th nucleotide and the 3′ end of the nucleotide sequence of SEQ ID NO:35 (codon-optimized nucleotide sequence of a gene encoding pyridoxamine-pyruvate transaminase from Skermanella stibiiresistens), the region between the 18th nucleotide and the 3′ end of the nucleotide sequence of SEQ ID NO:36 (codon-optimized nucleotide sequence of gene encoding pyridoxamine-pyruvate transaminase from Rhizobium sp. AC44/96), the region between the 18th nucleotide and the 3′ end of the nucleotide sequence of SEQ ID NO:37 (codon-optimized nucleotide sequence of a gene encoding pyridoxamine-pyruvate transaminase from Erwinia toletana), or the region between the 18th nucleotide and the 3′ end of the nucleotide sequence of SEQ ID NO:38 (codon-optimized nucleotide sequence of a gene encoding pyridoxamine-pyruvate transaminase from Herbiconiux ginsengi), and that encodes a protein having pyridoxamine synthetase activity.

The gene encoding the hydrogen peroxide-degrading enzyme may be, for example, DNA having a nucleotide sequence of SEQ ID NO:7 (the nucleotide sequence of a gene encoding a catalase from Listeria seeligeri), or DNA having a nucleotide sequence that hybridizes with DNA having a nucleotide sequence complementary to the nucleotide sequence of SEQ ID NO:7 under a stringent condition, and that encodes a protein having hydrogen peroxide-degrading enzymatic activity.

Alternatively, the gene encoding the hydrogen peroxide-degrading enzyme may be, for example, DNA having the nucleotide sequence of SEQ ID NO:7, or DNA having a nucleotide sequence in which one or more of the substitution, deletion, or insertion of a nucleotide, or the addition of an additional nucleotide to both or either of the 5′ end and the 3′ end of the nucleotide sequence are performed in the nucleotide sequence of SEQ ID NO:7, and which encodes a protein having hydrogen peroxide-degrading enzymatic activity. Examples of the degrees of the substitution, deletion, and insertion of a nucleotide, and the addition of an additional nucleotide to both or either of the N-terminus and C-terminus of the nucleotide sequence are as described above.

Alternatively, the hydrogen peroxide-degrading enzyme may be DNA having the nucleotide sequence of SEQ ID NO:7, or may be DNA having a nucleotide sequence that has, for example, 80% or more, 85% or more, 90% or more, or 95% or more sequence identity with the nucleotide sequence of SEQ ID NO:7, and that encodes a protein having hydrogen peroxide-degrading enzymatic activity.

### <Recombinant Microorganism Having Gene Encoding Pyridoxine Oxidase and Gene Encoding Pyridoxamine Synthetase>

In the recombinant microorganism according to the disclosure, each of a gene encoding a pyridoxine oxidase and a gene encoding a pyridoxamine synthetase may be endogenous to a bacterial cell and has an enhanced expression, or may be introduced from outside of a bacterial cell into the bacterial cell. Both of a gene endogenous to a bacterial cell and has an enhanced expression and a gene introduced from outside of a bacterial cell into the bacterial cell may exist in a recombinant microorganism. In the recombinant microorganism according to the disclosure, in addition to either or both of a gene that encodes a pyridoxine oxidase and is endogenous to a bacterial cell and has an enhanced expression and a gene that encodes a pyridoxine oxidase and is introduced from outside of a bacterial cell into the bacterial cell, a gene that encodes a non-enhanced (for example, promoter-unmodified) pyridoxine oxidase and is endogenous to a bacterial cell may exist. Similarly, in addition to either or both of a gene that encodes a pyridoxamine synthetase and is endogenous to a bacterial cell and has an enhanced expression and a gene that encodes a pyridoxamine synthetase and is introduced from outside of a bacterial cell into the bacterial cell, a gene that encodes a non-enhanced (for example, promoter-unmodified) pyridoxamine synthetase and is endogenous to the cell may be present. In the disclosure, since one or more of the enhancement of the expression of the gene endogenous to a bacterial cell and the introduction of the gene from outside of a bacterial cell into the bacterial cell are performed for each of the gene encoding a pyridoxine oxidase and the gene encoding a pyridoxamine synthetase, even when a gene whose expression is not enhanced originally exists in a bacterial cell, an expression level significantly higher than the expression level by the original gene whose expression is not enhanced can be obtained, whereby high pyridoxamine production efficiency can be achieved.

In a case in which the recombinant microorganism according to the disclosure further includes a gene encoding a hydrogen peroxide-degrading enzyme, the gene encoding the hydrogen peroxide-degrading enzyme may be endogenous to a bacterial cell, endogenous to a bacterial cell and has an enhanced expression, or introduced from outside of a bacterial cell into the bacterial cell. However, since the amount of hydrogen peroxide produced increases as the reaction rate increases, it is preferable to perform at least one of the enhancement of the expression of a gene that encodes a hydrogen peroxide-degrading enzyme and is endogenous to a bacterial cell or the introduction of a gene that encoding a hydrogen peroxide-degrading enzyme from outside of a bacterial cell into the bacterial cell, in order to increase the expression level of hydrogen peroxide-degrading enzyme.

In other words, each of the gene encoding a pyridoxine oxidase and the gene encoding a pyridoxamine synthetase may be endogenous to the genome of a host microorganism and has an enhanced expression by an operation such as the substitution of a promoter, or may be introduced from outside of a bacterial cell into the bacterial cell using a vector such as a plasmid. In addition to this, the recombinant microorganism may further include a gene endogenous to the genome of a host microorganism prior to the recombination and whose expression is not enhanced. In the disclosure, each of the gene encoding a pyridoxine oxidase and the gene encoding a pyridoxamine synthetase is introduced from outside of a cell in the host microorganism, or expression of the gene endogenous to the cell in the host microorganism is enhanced by the substitution of a promoter, or the like, whereby the expression of each of the genes is increased, and the pyridoxamine production capability due to the combination of the above-described two enzymes is further enhanced. In the case of performing the introduction or expression enhancement of each of the gene encoding a pyridoxine oxidase and the gene encoding a pyridoxamine synthetase, either or both of the introduction from outside of a cell in a host microorganism and the enhancement of the gene expression by the substitution of a promoter, or the like, may be performed.

The recombinant microorganism according to the disclosure may include a gene encoding a hydrogen peroxide-degrading enzyme, but it is not necessarily required. When the recombinant microorganism according to the disclosure further includes a gene encoding a hydrogen peroxide-degrading enzyme, the gene encoding a hydrogen peroxide-degrading enzyme may be endogenous to the genome of a host microorganism prior to recombination, endogenous to the genome of a host microorganism and has an enhanced expression by an operation such as the substitution of a promoter, or introduced from outside of a bacterial cell into the bacterial cell using a vector such as a plasmid. In the case of performing the introduction or expression enhancement of the gene encoding a hydrogen peroxide-degrading enzyme, either or both of the introduction from outside of a cell in a host microorganism and the enhancement of the gene expression by substitution of a promoter or the like may be performed.

It is impossible to obtain a sufficient production capability for highly producing pyridoxamine or a salt thereof unless such increase in expression as described above is performed in the gene encoding a pyridoxine oxidase and the gene encoding a pyridoxamine synthetase. Therefore, in the recombinant microorganism according to the disclosure, at least one of the introduction from outside of a bacterial cell or the enhancement of the expression of a gene endogenous to the bacterial cell is performed in each of the gene encoding a pyridoxine oxidase and the gene encoding a pyridoxamine synthetase. Introduction of an enzyme gene from outside of a bacterial cell is not necessarily limited to the introduction for compensating an enzyme gene which is not present in a host microorganism, and the introduction may be performed for the purpose of increasing the expression of an enzyme gene endogenous to a host microorganism. An enzyme gene which is not present in a host microorganism can be easily confirmed using an enzyme database such as KEGG or BRENDA.

In a case in which the expression of a gene endogenous to a host microorganism is enhanced by the substitution of a promoter of the gene with another promoter, the another promoter (newly introduced promoter) is not particularly limited as long as the promoter can enhance the gene expression (more than before promoter substitution) in the host microorganism, and may be a constitutive promoter or an inducible promoter. Substitution of a promoter can be performed using a general genetic modification technology. The sequence of a promoter endogenous to a host microorganism may be partially or completely left unless the sequence adversely affects expression by a newly introduced promoter in practical use.

When the host microorganism is, for example, a prokaryote, examples of the promoter that can be used as the newly introduced promoter include a trp promoter, a lac promoter, and a GAPDH promoter from E. coli, a PL promoter and a PR promoter from lambda phage, and a gluconate synthetase promoter (gnt), an alkaline protease promoter (apr), a neutral protease promoter (npr), and an α-amylase promoter (amy) from Bacillus subtilis. An originally modified or designed promoter such as a tac promoter can also be used.

When the host microorganism is, for example, a filamentous fungus, examples of the promoter that can be used as the newly introduced promoter include a cellobiohydrolase (cbh) promoter, an endoglucanase (egl) promoter, a xylanase III (xyn3) promoter, a U6 promoter, and an α-amylase (amy) promoter.

When the host microorganism is, for example, yeast, examples of the promoter that can be used as the newly introduced promoter include an alcohol dehydrogenase (ADH1) promoter, a phosphoglycerate kinase (PGK1) promoter, a peptide chain elongation factor (TEF) promoter, a glycerol 3-phosphate dehydrogenase (GPD) promoter, a galactokinase (GAL1) promoter, a metallothionein (CUP1) promoter, a repressive acid phosphatase (PHOS) promoter, and a glyceraldehyde 3-phosphate dehydrogenase (GAPDH) promoter. The sequences of the above-described promoters are not limited to those derived from yeast used as a host microorganism. An exogenous promoter such as a cytomegalovirus (CMV) promoter may also be used.

In the case of introducing a gene (heterogenous gene) from outside of a host microorganism, the method is not particularly limited as long as a gene can be introduced into the inside of a bacterial cell (inside a cell) and an enzyme encoded by the gene can be expressed, and examples thereof include transformation with a plasmid carrying an enzyme gene, introduction of an enzyme gene into a genome, and a combination thereof. When introducing a gene, an expression vector into which the gene is integrated may be introduced into a bacterial cell. The expression vector is not particularly limited as long as the vector is a vector into which the nucleotide sequence of the gene is integrated, and the expression vector is more preferably a plasmid vector or a phage vector having the following constitution, from the viewpoint of improving transformation efficiency or translation efficiency.

The expression vector is not particularly limited as long as the vector includes the nucleotide sequence of the gene and can transform the host microorganism. If necessary, in addition to such a nucleotide sequence, a nucleotide sequence (hereinafter, also simply referred to as “another region”) constituting another region may be included. Examples of such a region include a control region required to the production of a desired enzyme by a recombinant microorganism obtained by transformation, and a region required for autonomous replication.

From the viewpoint of facilitating selection of the recombinant microorganism, the vector may further include a nucleotide sequence encoding a selection gene which can be used as a selection marker.

Examples of a control region required to the production of a desired enzyme include a promoter sequence (including an operator sequence for controlling transcription), a ribosome-binding sequence (SD sequence), and a transcription termination sequence.

In the case of using yeast as a host microorganism, from the viewpoint of expression efficiency of the gene, an expression vector that can be used preferably includes a promoter sequence in addition to the nucleotide sequence of the above gene. Any promoter sequence may be used as long as the sequence can express the gene in a transformant obtained using yeast as a host microorganism. Examples of the promoter sequence include the promoters described above as examples of the promoter that can be used as the newly introduced promoter in the case of using yeast as a host microorganism.

The expression vector may include a secretion signal. This allows to, when a recombinant microorganism produces a desired enzyme, extracellular secretion of the enzyme.

The secretion signal is not particularly limited as long as a desired enzyme can be secreted from yeast as a host microorganism. From the viewpoint of secretion efficiency, it is preferable to use an α factor signal sequence, an invertase signal sequence, an acid phosphatase signal sequence, a glucoamylase signal sequence, or the like.

Specific examples of the expression vector having the promoter sequence and the secretion signal as described above include pRS423, pRS424, and YEplac195.

From the viewpoint of expression efficiency of the gene, an expression vector that can be used when a filamentous fungus is used as a host microorganism preferably includes a promoter sequence in addition to the nucleotide sequence of the above gene. Any promoter sequence may be used as long as the sequence can express the gene in a transformant obtained by using a filamentous fungus as a host microorganism. Examples of the promoter include the promoters described above as examples of the promoter that can be used as the newly introduced promoter in the case of using a filamentous fungus as the host microorganism.

Suitable expression vectors for filamentous fungi are described in van den Hondel, C. A. M. J. J. et al. (1991) In: Bennett, J. W. and Lasure, L. L. (eds.) More Gene Manipulations in Fungi. Academic Press, pp. 396-428.

Another commonly used expression vector such as pUC18, pBR322, pUC100, pSL1180 (manufactured by Pharmacia Inc.), pFB6 and Aspergillus pRAX, or Trichoderma pTEX can also be used.

From the viewpoint of the expression efficiency of the gene, an expression vector which can be used when a prokaryote such as E. coli, Bacillus subtilis, or Actinomycetes is used as a host microorganism preferably includes a promoter sequence in addition to the nucleotide sequence of the above gene. The expression vector may include a ribosome-binding sequence, a transcription termination sequence, or the like other than a promoter sequence.

Examples of the promoter sequence include the promoters described above as examples of the promoter that can be used as the newly introduced promoter in the case of using a prokaryote as the host microorganism.

Examples of the ribosome-binding sequence include a sequence derived from E. coli or B. subtilis, and the ribosome-binding sequence is not particularly limited thereto as long as the sequence is a sequence that functions in a desired host microorganism such as E. coli or B. subtilis.

Examples of the ribosome-binding sequence include a sequence in which a consensus sequence having four or more consecutive nucleotides, of sequences complementary to the 3′ end region of 16S ribosomal RNA is produced by DNA synthesis.

The transcription termination sequence is not necessarily required, and a ρ-factor independent transcription termination, for example, as a lipoprotein terminator, a trp operon terminator, or the like can be used.

The order of these sequences on the expression vector of the control region is not particularly limited, and the promoter sequence, the ribosome-binding sequence, the gene encoding a target enzyme, and the transcription termination sequence are desirably arranged in this order from the upstream (5′-end side) in consideration of transcription efficiency.

Specific examples of the expression vector that can be used when a host microorganism is a prokaryote include pBR322, pUC18, Bluescript II SK (+), pKK223-3, or pSC101 including a region that can be autonomously replicated in E. coli, or pUB110, pTZ4, pC194, ρ11, φ1, or φ105 including a region that can be autonomously replicated in Bacillus subtilis.

Examples of the vector that can be autonomously replicated in two or more kinds of host microorganisms include pHV14, TRp7, YEp7, and pBS7.

In the case of introducing a gene from outside of a cell in the host microorganism, the gene may be a gene having a nucleotide sequence that is not present in the genome of the host microorganism, and may be a gene having a nucleotide sequence that is present in the genome of the host microorganism. Even when the same gene originally exists in the genome of a host microorganism, the introduction of a gene from outside the bacterial cell causes stronger gene expression, and the enzymatic activity is enhanced and the production efficiency of pyridoxamine or a salt thereof can be further enhanced.

Conventional methods well known to those skilled in the art can be used as methods necessary for introducing a gene from outside of a bacterial cell (extracellular) into the bacterial cell (intracellular), such as preparation of a genomic DNA, cleavage and ligation of DNA, transformation, PCR (Polymerase Chain Reaction), and design and synthesis of an oligonucleotide used as a primer. These methods are described in Sambrook, J., et al., “Molecular Cloning A Laboratory Manual, Second Edition”, Cold Spring Harbor Laboratory Press, (1989) and the like. For example, a method using competent cells and a method using electroporation are described.

The host microorganism is not particularly limited as long as the microorganism is a microorganism capable of expressing, if present, a gene encoding a pyridoxine oxidase and a gene encoding a pyridoxamine synthetase. Examples of the host microorganism include yeasts, filamentous fungi, and prokaryotes. Examples of the yeasts include a yeast belonging to the genus Saccharomyces, such as Saccharomyces cerevisiae, a yeast belonging to the genus Schizosaccharomyces, such as Schizosaccharomyces pombe, a yeast belonging to the genus Hansenula, and a yeast belonging to the genus Pichia. Examples of the filamentous fungi include a filamentous fungus belonging to the genus Trichoderma, such as Trichoderma reesei or viride, a filamentous fungus belonging to the genus Aspergillus, such as Aspergillus niger or oryzae, a filamentous fungus belonging to the genus Humicola, such as Humicola insolens, and a filamentous fungus belonging to the genus Acremonium, such as Acremonium cellulolyticus orfusidioides. Examples of the prokaryotes include a bacterium belonging to the genus Escherichia, such as Escherichia coli, a bacterium belonging to the genus Schewanella, such as Schewanella sp. AC10, a bacterium belonging to the genus Mesorhizobium, such as Mesorhizobium loti, a bacterium belonging to the genus Rhizobium, such as Rhizobium meliloti, a Bacillus bacterium belonging to the genus Bacillus, such as Bacillus subtilis, and an actinomycete such as an actinomycete belonging to the genus Streptomyces, such as Streptomyces lividans.

A desired gene can be introduced into a host microorganism by, for example, introducing (transforming) into these host microorganisms an expression vector including the desired gene. The introduced gene can be highly expressed in the obtained recombinant microorganism, for example, by the activity of a promoter included in the expression vector.

In a case in which the host microorganism is E. coli, for example, a competent cell method by calcium treatment, an electroporation method, or the like can be used as a method of introducing the recombinant DNA such as an expression vector into a cell of the host microorganism. The enzyme encoded by the introduced gene can be stably produced at a high expression level by culturing a recombinant microorganism obtained in such a manner.

The DNA encoding the enzyme can be isolated from the recombinant microorganism, and can be transferred into another microorganism. Using this DNA as a template, the DNA encoding the enzyme can be easily introduced into another host microorganism, by amplifying a DNA fragment encoding an enzyme by PCR, treating the resultant with a restriction enzyme or the like, and then the obtained fragment is ligated with another vector DNA fragment.

<Method of Producing Pyridoxamine or Salt Thereof>

In one embodiment, a method of producing pyridoxamine or a salt thereof includes bringing the recombinant microorganism, the culture of the recombinant microorganism, or the treated product of the recombinant microorganism or the culture according to the disclosure, into contact with pyridoxine or a salt thereof in the presence of oxygen to produce pyridoxamine or a salt thereof.

Examples of a salt of pyridoxine include a salt of pyridoxine and an acid. Examples of the acid include hydrochloric acid, sulfuric acid, nitric acid, and phosphoric acid. Examples of the salt of pyridoxine include pyridoxine hydrochloride.

Examples of the salt of pyridoxamine include a salt of pyridoxamine with an acid. Examples of the acid include hydrochloric acid, sulfuric acid, nitric acid, acetic acid, and phosphoric acid. From the viewpoint of use as a medicament, the salt of pyridoxamine is preferably pyridoxamine dihydrochloride, which is most advanced for medical applications.

Such a method of producing pyridoxamine or a salt thereof using the recombinant microorganism, the culture of the recombinant microorganism, or the treated product of the recombinant microorganism or the culture is also simply referred to as “method of producing pyridoxamine or a salt thereof using a recombinant microorganism”.

The culture of the recombinant microorganism refers to a product obtained by culturing the recombinant microorganism, which is composed of a bacterial cell, a surrounding medium, and the like. In a case in which an enzyme is secreted extracellularly, the enzyme can more easily contact with a substrate when such a culture is used, and the efficiency of producing pyridoxamine or a salt thereof can be improved. The use of the culture is not necessarily required. For example, a dried or frozen recombinant microorganism cell prepared in advance may be added directly to a reaction system.

Any of a synthetic medium and a natural medium can be used as a culture medium in culturing a recombinant microorganism, as long as the culture medium is a culture medium including adequate amounts of a carbon source, a nitrogen source, an inorganic substance, and another nutrient. The culture can be performed using a general culture method, such as shaking culture, aeration stirring culture, continuous culture, or fed-batch culture in a liquid medium including the culture components.

More specifically, the culture condition for the recombinant microorganism is the same as the culture condition for the original host microorganism, and known conditions can be used.

Known components can be used as the components used in the culture medium. For example, organic nutrient sources such as a meat extract, a yeast extract, a malt extract, peptone, NZ amine, and potato, carbon sources such as glucose, maltose, sucrose, starch, and an organic acid, nitrogen sources such as ammonium sulfate, urea, and ammonium chloride, inorganic nutrient sourced such as phosphate, magnesium, potassium, and iron, and vitamins can be used in combination, if appropriate.

In culture of a recombinant microorganism transformed by the expression vector including the selection marker, for example, a culture medium including an agent against the drug resistance is used in a case in which the selection marker has drug resistance, and a culture medium that does not include a nutrient is used in a case in which the selection marker has a requirement for the nutrient.

The culture conditions may be selected, if appropriate, depending on the kinds of the recombinant microorganism, a culture medium, and a culture method, and are not particularly limited as long as the culture conditions are conditions under which the recombinant microorganism grows and can produce a pyridoxine oxidase and a pyridoxamine synthetase.

The pH of the culture medium may be selected, for example, in a range of from pH 4 to pH 8, and may be in a range of from pH 5 to pH 8.

The culture temperature is, for example, from 20° C. to 45° C., and preferably from 24° C. to 37° C. The culture may be performed aerobically or anaerobically depending on the kinds of a microorganism.

The culture period is, for example, from 1 day to 7 days. The culture period may be set to maximize the production amount of the target enzyme.

The treated product of the recombinant microorganism refers to a product obtained by any treatment of the recombinant microorganism as long as the activities of the pyridoxine oxidase and the pyridoxamine synthetase produced by the recombinant microorganism are not lost. Examples of such treatment include one or more selected from the group consisting of heat treatment, cooling treatment, mechanical destruction, ultrasonic treatment, freeze-thaw treatment, drying treatment, pressurized or reduced pressure treatment, osmotic pressure treatment, autolysis, surfactant treatment, and enzyme treatment (for example, cell lysis treatment). Even in a case in which the recombinant microorganism per se is killed by such treatment, such a treated product can be used for a reaction as long as the activity of the enzyme produced by the microorganism remains.

The treated product of the culture refers to a product obtained by any treatment of the culture of the recombinant microorganism as long as the activities of the pyridoxine oxidase and the pyridoxamine synthetase produced by the recombinant microorganism are not lost. Examples of such treatment include one or more selected from the group consisting of heat treatment, cooling treatment, mechanical destruction of a cell, ultrasonic treatment, freeze-thaw treatment, drying treatment, pressurized or reduced pressure treatment, osmotic pressure treatment, cell autolysis, surfactant treatment, enzyme treatment (for example, cell destruction treatment), cell separation treatment, purification treatment, and extraction treatment. For example, a cell of the recombinant microorganism may be separated from a culture medium or the like, and the separated cell may be added to a reaction system. Means such as filtration or centrifugation can be used for such separation. Alternatively, purification treatment for separating, from a contaminant, the pyridoxine oxidase and the pyridoxamine synthetase, and, if present, the hydrogen peroxide-degrading enzyme, may be performed, and a solution including an enzyme obtained by the purification treatment may be added to the reaction system. Alternatively, an extract obtained by extracting the culture using an organic solvent such as methanol or acetonitrile, or a mixed solvent of an organic solvent and water may be added to the reaction system. It is also acceptable that such a purified product or extract does not include a cell of a recombinant microorganism. Even in a case in which the cell of the microorganism does not exist, such a purified product or extract can be used for a reaction as long as the enzymatic activity remains.

Such disruption or lysis treatment of a cell as described above can be performed by destructing the cell membrane of the recombinant microorganism according to a known method such as lysozyme treatment, freeze-thawing, or ultrasonic disruption.

It is preferable that the recombinant microorganism, the culture of the recombinant microorganism, or the treated product of the recombinant microorganism or the culture according to the disclosure is brought into contact with pyridoxine or a salt thereof under the following conditions.

The contact is preferably performed in a solution including pyridoxine or a salt thereof as a substrate. The pH of the solution is not particularly limited as long as the enzymatic activities of the pyridoxine oxidase and the pyridoxamine synthetase are maintained, and is preferably from pH 6.0 to pH 9.0, and more preferably from pH 7.0 to pH 8.5. The temperature of the solution is also not particularly limited as long as the enzymatic activities of the pyridoxine oxidase and the pyridoxamine synthetase are maintained, and is preferably from 20° C. to 70° C., and more preferably from 25° C. to 50° C.

As a medium for the solution, water or an aqueous medium, an organic solvent, or a mixed solvent of an organic solvent and water or an aqueous medium may be used. Examples of the aqueous medium include buffers such as a phosphate buffer, a HEPES (N-2-hydroxyethylpiperazine-N-ethanesulfonic acid) buffer, and a tris[tris(hydroxymethyl)aminomethane]hydrochloride buffer. Any organic solvent may be used as long as the solvent does not inhibit a reaction, and examples thereof include acetone, ethyl acetate, dimethyl sulfoxide, xylene, methanol, ethanol, and butanol.

The recombinant microorganism, the culture of the recombinant microorganism, or the treated product of the recombinant microorganism or the culture is brought into contact with pyridoxine or a salt thereof in the presence of oxygen. This is because the pyridoxine oxidase consumes oxygen when oxidizing pyridoxine or a salt thereof. Regarding the oxygen concentration in a reaction system, for example, a reaction can be conducted while a solution including the recombinant microorganism, the culture of the recombinant microorganism, or the treated product of the recombinant microorganism or the culture, and pyridoxine or a salt thereof is opened to the atmosphere, in other words, the solution is brought into contact with the atmosphere, or while the solution is brought into contact with a gas including from 0.1% to 20%, from 0.5% to 10%, or from 1% to 5% of oxygen by volume. The amount of dissolved oxygen in the solution may be from 0.1 mg to 13 mg oxygen/L, from 0.5 mg to 10 mg oxygen/L, or from 1 mg to 8 mg oxygen/L.

In a case in which the recombinant microorganism has a gene encoding a hydrogen peroxide-degrading enzyme capable of generating oxygen, the reaction can be conducted under a condition in which the oxygen concentration is reduced or a condition in which oxygen supply is reduced or blocked. For example, it is also possible to carry out the reaction under a condition in which a reaction system is sealed or under a condition in which a reaction system is purged with nitrogen (nitrogen substituted).

It is preferable that the recombinant microorganism, the culture of the recombinant microorganism, or the treated product of the recombinant microorganism or the culture according to the disclosure is brought into contact with pyridoxine or a salt thereof under a shaking or stirring condition. For example, such contact can be performed in a solution. For example, pyridoxine or a salt thereof may be added to a solution including the recombinant microorganism, the culture of the recombinant microorganism, or the treated product of the recombinant microorganism or the culture in the form of a substrate solution or in the form of a solid. An amino acid to be consumed by a pyridoxamine synthetase may be further added to a solution including the recombinant microorganism, the culture of the recombinant microorganism, or the treated product of the recombinant microorganism or the culture. The amino acid may be added, in a state of being included in a substrate solution containing pyridoxine or a salt thereof, to a solution including the recombinant microorganism, the culture of the recombinant microorganism, or the treated product of the recombinant microorganism or the culture together with the pyridoxine or a salt thereof. Alternatively, the amino acid may be added, in a state of being included in a substrate solution other than the substrate solution containing pyridoxine or a salt thereof or in the form of a solid, to a solution including the recombinant microorganism, the culture of the recombinant microorganism, or the treated product of the recombinant microorganism or the culture.

An acid or an alkali may be added to maintain the pH of a reaction liquid in an appropriate range at the initiation of the reaction or during the reaction. Examples of the alkali that can be added to the reaction liquid include an alkali metal hydroxide such as lithium hydroxide, sodium hydroxide, or potassium hydroxide; and one such as ammonium hydroxide, calcium hydroxide, dipotassium phosphate, disodium phosphate, potassium pyrophosphate, or ammonia which is dissolved in water to make the liquid basic. Examples of the acid that can be added to a reaction liquid include hydrochloric acid, sulfuric acid, nitric acid, acetic acid, and phosphoric acid.

The contact may be performed, for example, under an air atmosphere or under a partial deoxygenation atmosphere. The deoxidizing atmosphere can be achieved by substitution with an inert gas, pressure reduction, boiling, or a combination thereof. It is preferable at least to replace with an inert gas, or to use an inert gas atmosphere. Examples of the inert gas include nitrogen gas, helium gas, argon gas, and carbon dioxide, and nitrogen gas is preferable. Since the pyridoxine oxidase consumes oxygen when oxidizing pyridoxine, an atmosphere in which oxygen is not removed is preferable.

In a preferable embodiment, the recombinant microorganism, the culture of the recombinant microorganism, or the treated product of the recombinant microorganism or the culture to be used includes the pyridoxine oxidase and the pyridoxamine synthetase. For this reason, the pyridoxine oxidase and the pyridoxamine synthetase, which are present together in the reaction liquid, act cooperatively when contacting, and pyridoxamine or a salt thereof is produced with high production efficiency. Although it is not essential that the treated product of the recombinant microorganism or the treated product of the culture includes the recombinant microorganism in a living state, from the viewpoint of continuously supplying the substance involved in the reaction by metabolism, it is preferable to include the recombinant microorganism in a living state.

Regarding the timing of addition, the recombinant microorganism, the culture of the recombinant microorganism, or the treated product of the recombinant microorganism or the culture may be added all at once at the initiation of a reaction, or may be added in batches or continuously during a reaction. Similarly, pyridoxamine, which is a raw material, may be added all at once at the initiation of a reaction, or may be added in batches or continuously during a reaction.

The reaction liquid may include an amino acid to be consumed in the production of pyridoxamine or a salt thereof by a pyridoxamine synthetase. This amino acid may also be added all at once at the initiation of a reaction, or may be added in batches or continuously during a reaction. For example, in a case in which a pyridoxamine-pyruvate transaminase is used as a pyridoxamine synthetase, the reaction liquid may include one or more of L-alanine and D-alanine. In a case in which a pyridoxamine-oxaloacetate transaminase or an aspartate transaminase is used as a pyridoxamine synthetase, the reaction liquid may include at least one of L-aspartic acid, D-aspartic acid, L-glutamic acid, or D-glutamic acid. An amino acid may also exist as a salt depending on a surrounding environment; however, in the disclosure, a description of amino acid is given with inclusion of the mention of a salt. For example, the description of L-glutamic acid includes not only L-glutamic acid which has not formed salt, but also L-glutamic acid which has formed salt (for example, monosodium L-glutamate monohydrate). Examples of counter ions in the case of forming a salt include cations such as sodium ion and potassium ion, and anions such as chloride ion, acetate ion, and nitrate ion.

The concentration of pyridoxine or a salt thereof in the reaction liquid may be, for example, from 0.1 mM to 500 mM, or may be from 0.4 mM to 200 mM, from 0.5 mM to 100 mM, or from 0.8 mM to 50 mM. The enzymatic activity of pyridoxine oxidase tends to be inhibited when the concentration of pyridoxine or a salt thereof is increased. For this reason, it is preferable that the concentration of pyridoxine or a salt thereof in the reaction liquid is not excessively high. In other words, the concentration of pyridoxine or a salt thereof in the reaction liquid may be controlled, for example, to maintain the concentration within the above range. For example, the method of producing pyridoxamine or a salt thereof according to the disclosure may include either or both of the following (A) and (B):

(A) adding pyridoxine or a salt thereof continuously or in several batches to a solution including the recombinant microorganism, the culture of the recombinant microorganism, or the treated product of the recombinant microorganism or the culture; and

(B) controlling a molar concentration of an amino acid consumed by the pyridoxamine synthetase so as to be 1 or more times a molar concentration of pyridoxine or a salt thereof, in a solution including the recombinant microorganism, the culture of the recombinant microorganism, or the treated product of the recombinant microorganism or the culture.

Examples of the amino acids include L-alanine, D-alanine, L-glutamic acid, D-glutamic acid, L-aspartic acid, and D-aspartic acid.

For such control, for example, pyridoxine or a salt thereof may be added in several batches instead of being added all at once to the reaction liquid. Since the concentration of pyridoxine decreases as a reaction proceeds, excessively high concentrations of pyridoxine or a salt thereof can be avoided by adding pyridoxine or a salt thereof at intervals. Examples of such sequential addition include, for example, addition of pyridoxine or a salt thereof twice or more, preferably three or more times, at a time interval in the range of from 0.5 hours to 10 hours. Pyridoxine or a salt thereof may be continuously added to the reaction liquid. In the case of continuous addition, excessively high concentrations of pyridoxine or a salt thereof can be avoided by adjusting the addition rate. For this adjustment, the concentration of pyridoxine or a salt thereof in the reaction liquid may be measured at a specific timing or may be monitored continuously.

The concentration of the amino acid (the amino acid to be consumed in producing pyridoxamine or a salt thereof by a pyridoxamine synthetase) may be, for example, from 0.1 mM to 2,000 mM, or may be from 0.2 mM to 1,000 mM, from 0.4 mM to 500 mM, from 0.5 mM to 400 mM, from 1 mM to 300 mM, or from 2 mM to 250 mM. The method for adding the amino acid is not particularly limited, and the amino acid may be added all at once, may be added in several batches, or may be added continuously. Examples of such sequential addition include, for example, adding an amino acid twice or more, preferably three or more times at a time interval in the range of from 0.5 hours to 10 hours. In a case in which pyridoxine or a salt thereof is added in portions, the amino acid may be added simultaneously with the addition of pyridoxine or a salt thereof, or may be added separately from pyridoxine or a salt thereof.

The concentration (molar concentration) of the amino acid (the amino acid consumed when pyridoxamine or a salt thereof is produced by pyridoxamine synthetase) is preferably maintained higher than the concentration (molar concentration) of pyridoxine or a salt thereof. In a case in which the concentration of the amino acid is maintained higher than the concentration of pyridoxine or a salt thereof, the reaction equilibrium can be shifted in favor of pyridoxamine or a salt thereof, and the production efficiency of pyridoxamine or a salt thereof can be further improved. The molar concentration of the amino acid is preferably 1.0 time or more, more preferably 1.5 times or more, still more preferably 2.0 times or more, and still more preferably 5.0 times or more, and may be 10.0 times or more the molar concentration of pyridoxine or a salt thereof.

Examples of the method of bringing the recombinant microorganism, the culture of the recombinant microorganism, or the treated product of the recombinant microorganism or the culture into contact with pyridoxine or a salt thereof include: a method of adding the recombinant microorganism, the culture of the recombinant microorganism, or the treated product of the recombinant microorganism or the culture to a solution including pyridoxine or a salt thereof and allowing a reaction to proceed while stirring; a method of adding the recombinant microorganism, the culture of the recombinant microorganism, or the treated product of the recombinant microorganism or the culture to a solution including pyridoxine or a salt thereof and allowing a reaction to proceed while shaking; and a method of mixing the recombinant microorganism, the culture of the recombinant microorganism, or the treated product of the recombinant microorganism or the culture and pyridoxine or a salt thereof thoroughly in a solution, then allowing the mixture to stand still for allowing a reaction to proceed. From the viewpoint of reaction efficiency, preferable examples thereof include a method of adding the recombinant microorganism, the culture of the recombinant microorganism, or the treated product of the recombinant microorganism or the culture to a solution including pyridoxine or a salt thereof and allowing a reaction to proceed while stirring.

A reaction vessel that can be used for a reaction is not particularly limited. The reaction vessel is preferably a vessel in which the recombinant microorganism, the culture of the recombinant microorganism, or the treated product of the recombinant microorganism or the culture and the solution including pyridoxine or a salt thereof added thereto can be stirred enough to mix them sufficiently, and which has a temperature control function of keeping the temperature within an optimum temperature range for a pyridoxine oxidase and a pyridoxamine synthetase.

The contact time (reaction time) of the recombinant microorganism, the culture of the recombinant microorganism, or the treated product of the recombinant microorganism or the culture with pyridoxine or a salt thereof is not particularly limited as long as the enzymatic activities of the pyridoxine oxidase and the pyridoxamine synthetase are maintained. For example, the contact time may be from 30 minutes to 100 hours, and may be from 2 hours to 50 hours. The reaction may be performed with a batch method, may be performed with a semi-batch method in which either or both of a substrate, and the microorganism, the culture or the treated product are added in batches during the reaction, or may be performed with a continuous method. In the case of the semi-batch method or the continuous method, the upper limit of the reaction time is not particularly limited since an operation such as supplying either or both of a new raw material, and the recombinant microorganism, the culture, or the treated product is conducted.

In the above method, the use of the recombinant microorganism, the culture of the recombinant microorganism, or the treated product of the recombinant microorganism or the culture according to the disclosure, allows the production of pyridoxamine or a salt thereof inexpensively with high production efficiency by using pyridoxine or a salt thereof as a raw material, without the need to carry out a complicated step as in a chemical synthesis method. Pyridoxamine or a salt thereof obtained by the above-described method can be used, for example, for producing a product that utilizes its physiological activity. For example, the product can be used for an application such as prevention or treatment of a disease or schizophrenia caused by AGE accumulation such as diabetes mellitus, atherosclerosis, chronic renal failure, or Alzheimer's dementia, a health food, or a cosmetic.

The term “step” as used herein includes not only a separate step but also a step that is not clearly distinguished from other steps as long as the desired purpose of the step is achieved therefrom. As used herein, the notation “to” expressing a numerical range including the numerical values before and after “to”, as the minimum value and the maximum value, respectively.

Herein, the amount of a component of a composition, when plural substances corresponding to the same component exist in the composition, the total amount of the component in the composition refers to a total amount of the plural substances in the composition, unless otherwise specified.

### EXAMPLES

Hereinafter, the present embodiment is described more specifically by referring to the following examples, but the disclosure is not limited at all to these examples. In addition, “%” indicating the amounts of components in compositions in examples is based on mass standard unless otherwise specified.

<Analysis Conditions>

Pyridoxine hydrochloride, pyridoxal hydrochloride, and pyridoxamine dihydrochloride were quantified by high performance liquid chromatography. The analysis conditions thereof are as follows.

Column: Shodex (registered trademark) Asahipak ODP-50 6E (SHOWA DENKO K.K.)

Guard column: Shodex (registered trademark) Asahipak ODP-50G 6A (SHOWA DENKO K.K.)

Column temperature: 30° C.

Pump flow rate: 1.0 mL/min

Eluent: 50 mM phosphate buffer (pH 2.0)

Detection: UV 254 nm

### Comparative Example 1: Production of pno Expression Strain

A synthetic DNA having the nucleotide sequence of SEQ ID NO:9 was obtained by custom synthesis of the nucleotide sequence of a pyridoxine 4-oxidase gene derived from Microbacterium luteolum, codon-optimized for E. coli, from GenScript. A DNA fragment including a target gene was amplified by PCR with an oligonucleotide having the nucleotide sequence of SEQ ID NO:12 and an oligonucleotide having the nucleotide sequence of SEQ ID NO:13 as primers using the synthetic DNA as a template. The amplified DNA fragment was treated with BamHI and SalI, and the obtained DNA fragment and a treated product of pUC18 (manufactured by Takara Bio Inc.) with BamHI and SalI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:9 according to a usual method of determining a nucleotide sequence. The obtained plasmid was named pUC18-pno. Here, pno is the abbreviated name of a pyridoxine-4-oxidase.

### Example 1: Production of ppat-pno Expression Strain

A synthetic DNA having the nucleotide sequence of SEQ ID NO:10 was obtained by custom synthesis of the nucleotide sequence of a pyridoxamine-pyruvate aminotransferase gene derived from Mesorhizobium loti MAFF303099, codon-optimized for E. coli, from GenScript. A DNA fragment including a target gene was amplified by PCR with an oligonucleotide having the nucleotide sequence of SEQ ID NO:14 and an oligonucleotide having the nucleotide sequence of SEQ ID NO:15 as primers using the synthetic DNA as a template. The amplified DNA fragment was treated with EcoRI and BamHI, and the obtained DNA fragment and a treated product obtained by treating the pUC18-pno produced in Comparative Example 1 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:10 according to a usual method of determining a nucleotide sequence. The obtained plasmid was named pUC18-ppat-pno. Here, ppat is the abbreviated name of pyridoxamine-pyruvate aminotransferase.

### Example 2: Production of ppat-pno-kat Expression Strain

A synthetic DNA having the nucleotide sequence of SEQ ID NO:11 was obtained by custom synthesis of the nucleotide sequence of a catalase gene derived from Listeria seeligeri, codon-optimized for E. coli, from GenScript. The synthetic DNA was treated with SalI and HindIII, and the obtained DNA fragment and a treated product obtained by treating the pUC18-ppat-pno produced in Example 1 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner. Here, kat is the abbreviated name of a catalase.

The nucleotide sequence of the DNA fragment introduced into the plasmid was confirmed to be the nucleotide sequence of SEQ ID NO:11 according to a usual method of determining a nucleotide sequence. The obtained plasmid was named pUC18-ppat-pno-kat.

### Example 3: Production of aspC-pno Expression Strain

A DNA fragment including a pyridoxamine-oxaloacetate transaminase gene derived from Escherichia coli W3110 was amplified by PCR with an oligonucleotide having the nucleotide sequence of SEQ ID NO:16 and an oligonucleotide having the nucleotide sequence of SEQ ID NO:17 as primers using the genomic DNA of Escherichia coli W3110 as a template. The amplified DNA fragment was treated with EcoRI and BamHI, and the obtained DNA fragment and a treated product obtained by treating the pUC18-pno produced in Example 1 with EcoRI and BamHI were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:8 according to a usual method of determining a nucleotide sequence. The obtained plasmid was named pUC18-aspC-pno. Here, aspC is the abbreviated name of pyridoxamine-oxaloacetate transaminase.

### Example 4: Production of aspC-pno-kat Expression Strain

A synthetic DNA having the nucleotide sequence of SEQ ID NO:11 was obtained by custom synthesis of the nucleotide sequence of a catalase gene derived from Listeria seeligeri, codon-optimized for E. coli, from GenScript. The synthetic DNA was treated with SalI and HindIII, and the obtained DNA fragment and a treated product obtained by treating the pUC18-aspC-pno produced in Example 3 with SalI and HindIII were ligated using Ligation High (manufactured by TOYOBO Co., Ltd.). Escherichia coli DH5a (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner. Here, kat is the abbreviated name of a catalase.

The nucleotide sequence of the DNA fragment introduced into the plasmid was confirmed to be the nucleotide sequence of SEQ ID NO:11 according to a usual method of determining a nucleotide sequence. The obtained plasmid was named aspC-pno-kat.

Test 1: Production of Pyridoxamine Using Pyridoxine as Raw Material

DH5α transformed with each of pUC18 and the plasmids produced in Comparative Example 1 and Example 1 was inoculated into 100 mL of LB medium including 100 μg/mL of ampicillin in a 500 mL baffled Erlenmeyer flask and cultured with shaking at 30° C. for 24 hours. The culture solution was centrifuged at 8,000 rpm for 20 minutes, and bacterial cells obtained as a precipitate were suspended in 800 μL of water to prepare a bacterial cell suspension.

Pyridoxine hydrochloride and L-alanine were dissolved in water to prepare a substrate solution including pyridoxine hydrochloride (200 mM) and L-alanine (800 mM). The pH of the substrate solution was adjusted to pH 8.0 with sodium hydroxide. 100 μL of substrate solution and 300 μL of bacterial cell suspension were mixed in a 2.0 mL tube, and the mixture was shaken at 1,000 rpm and 37° C. for 6 hours while the tube was uncapped. A part of the reaction liquid was collected, and analyzed under the analysis conditions described above. The reaction yield obtained as a result is set forth in Table 1. The yield set forth in Table 1 represents the ratio of the molar amount of obtained pyridoxamine dihydrochloride to the molar amount of pyridoxine hydrochloride in the substrate solution.

TABLE 1

| Plasmid | Yield |
|---|---|
| pUC18 | 0% |
| pUC18-pno | 23% |
| pUC18-ppat-pno | 84% |

As set forth in Table 1, pyridoxamine dihydrochloride was produced with high production efficiency in a case in which both of the gene encoding pyridoxine oxidase and the gene encoding pyridoxamine synthetase were extracellularly introduced. In the transformant including the plasmid produced in Comparative Example 1, it is assumed that a spontaneous reaction between pyridoxal and an amino group donor occurred. As described above, in the case of this spontaneous reaction, it is impossible to achieve high pyridoxamine production efficiency due to formation of a by-product. Also in the results of Table 1, when a transformant including the plasmid produced in Comparative Example 1 was used, high pyridoxamine production efficiency was not achieved.

Test 2: Production of Pyridoxamine Using Pyridoxine as Raw Material

DH5α transformed with each of pUC18 and the plasmid produced in Example 3 was inoculated into 100 mL of LB medium including 100 μg/mL of ampicillin in a 500 mL baffled Erlenmeyer flask and cultured with shaking at 30° C. for 24 hours. The culture solution was centrifuged at 8,000 rpm for 20 minutes, and bacterial cells obtained as a precipitate were suspended in 800 μL of water to prepare a bacterial cell suspension.

Pyridoxine hydrochloride and monosodium L-glutamate monohydrate were dissolved in water to prepare a substrate solution including pyridoxine hydrochloride (200 mM) and monosodium L-glutamate monohydrate (800 mM). The pH of the substrate solution was adjusted to pH 8.0 with sodium hydroxide. 300 μL of substrate solution and 900 μL of bacterial cell suspension were mixed in a 2.0 mL tube, and the mixture was shaken at 1,000 rpm and 37° C. for 24 hours while the tube was uncapped. A part of the reaction liquid was collected, and analyzed under the analysis conditions described above. The reaction yield obtained as a result is set forth in Table 2. The yield set forth in Table 2 represents the ratio of the molar amount of obtained pyridoxamine dihydrochloride to the molar amount of pyridoxine hydrochloride in the substrate solution.

TABLE 2

| Plasmid | Yield |
|---|---|
| pUC18 | 0% |
| pUC18-acpC-pno | 60% |

As set forth in Table 2, pyridoxamine dihydrochloride was produced with high production efficiency in a case in which the gene encoding pyridoxine oxidase and the gene encoding pyridoxamine synthetase were extracellularly introduced.

Test 3: Production of Pyridoxamine Using Pyridoxine as Raw Material (Examination of Oxygen Concentration)

DH5α transformed with the plasmid (pUC18-ppat-pno) produced in Example 1 was inoculated into 100 mL of LB medium including 100 μg/mL of ampicillin in a 500 mL baffled Erlenmeyer flask, and cultured with shaking at 30° C. for 24 hours. The culture solution was centrifuged at 8,000 rpm for 20 minutes, and bacterial cells obtained as a precipitate were suspended in 800 μL of water to prepare a bacterial cell suspension.

Pyridoxine hydrochloride and L-alanine were dissolved in water to prepare a substrate solution including pyridoxine hydrochloride (200 mM) and L-alanine (800 mM). The pH of the substrate solution was adjusted to pH 8.0 with sodium hydroxide. 100 μL of substrate solution and 300 μL of bacterial cell suspension were mixed in a 2.0 mL tube. The reaction was allowed to proceed with shaking at 1,000 rpm and 37° C. for 24 hours under a condition in which the tube was uncapped, under a condition in which the tube was capped, or under a condition in which the tube was capped after purged with nitrogen. A part of the reaction liquid was collected, and analyzed under the analysis conditions described above. The reaction yield obtained as a result is set forth in Table 3. The yield set forth in Table 3 represents the ratio of the molar amount of obtained pyridoxamine dihydrochloride to the molar amount of pyridoxine hydrochloride in the substrate solution.

TABLE 3

| Plasmid | Yield |
|---|---|
| In presence of air (Open system) | 84% |
| In presence of air (Closed system) | 21% |
| Purged with nitrogen (Closed system) | 2% |

As set forth in Table 3, pyridoxamine or a salt thereof was produced at high yield under a condition in which oxygen supply was not limited than a condition in which oxygen supply was limited, in a case in which the recombinant microorganism into which the gene encoding pyridoxine oxidase and the gene encoding pyridoxamine synthetase had been extracellularly introduced was used to produce pyridoxamine or a salt thereof.

Test 4: Production of Pyridoxamine Using Pyridoxine as Raw Material (Examination of Effect by Hydrogen Peroxide-Degrading Enzyme)

DH5α transformed with each of the plasmid (pUC18-ppat-pno) produced in Example 1 and the plasmid (pUC18-ppat-pno-kat) produced in Example 2 was inoculated into 100 mL of LB medium including 100 μg/mL of ampicillin in a 500 mL baffled Erlenmeyer 1 flask, and cultured with shaking at 30° C. for 24 hours. The culture solution was centrifuged at 8,000 rpm for 20 minutes, and bacterial cells obtained as a precipitate were suspended in 800 μL of water to prepare a bacterial cell suspension.

Pyridoxine hydrochloride and L-alanine were dissolved in water to prepare a substrate solution including pyridoxine hydrochloride (200 mM) and L-alanine (800 mM). The pH of the substrate solution was adjusted to pH 8.0 with sodium hydroxide. 100 μL of substrate solution and 300 μL of bacterial cell suspension were mixed in a 2.0 mL tube. The reaction was allowed to react with shaking at 1,000 rpm for 24 hours at 37° C. while the tube was capped. A part of the reaction liquid was collected, and analyzed under the analysis conditions described above. Table 4 shows the relative value (ratio of PM production rate) of the amount of pyridoxamine dihydrochloride produced using DH5α transformed with pUC18-ppat-pno-kat, with the amount of pyridoxamine dihydrochloride produced using DH5α transformed with pUC18-ppat-pno set to 1.0.

TABLE 4

| Plasmid | Ratio of PM Production Rate |
|---|---|
| pUC18-ppat-pno | 1.0 |
| pUC18-ppat-pno-kat | 1.4 |

As set forth in Table 4, it is understood that in a case in which the recombinant microorganism according to the disclosure further includes the gene encoding a hydrogen peroxide-degrading enzyme, the production efficiency of pyridoxamine or a salt thereof under a condition in which oxygen supply is limited is further improved.

Test 5: Production of Pyridoxamine Using Pyridoxine as Raw Material (Examination of Effect by Hydrogen Peroxide-Degrading Enzyme)

DH5α transformed with each of the plasmid (pUC18-aspC-pno) produced in Example 3 and the plasmid (pUC18-aspC-pno-kat) produced in Example 4 was inoculated into 100 mL of LB medium including 100 μg/mL of ampicillin in a 500 mL baffled Erlenmeyer flask and cultured with shaking at 30° C. for 24 hours. The culture solution was centrifuged at 8,000 rpm for 20 minutes, and bacterial cells obtained as a precipitate were suspended in 800 μL of water to prepare a bacterial cell suspension.

Pyridoxine hydrochloride and monosodium L-glutamate monohydrate were dissolved in water to prepare a substrate solution including pyridoxine hydrochloride (200 mM) and monosodium L-glutamate monohydrate (800 mM). The pH of the substrate solution was adjusted to pH 8.0 with sodium hydroxide. 100 μL of the substrate solution and 300 μL of the bacterial cell suspension were mixed in a 2.0 mL tube. The reaction was allowed to proceed with shaking at 1,000 rpm for 24 hours at 37° C. while the tube was capped. A part of the reaction liquid was collected, and analyzed under the analysis conditions described above. Table 5 shows the relative value (PM production rate ratio) of the amount of pyridoxamine dihydrochloride produced using DH5α transformed with pUC18-aspC-pno-kat, with the amount of pyridoxamine dihydrochloride produced using DH5α transformed with pUC18-aspC-pno set to 1.0.

TABLE 5

| Plasmid | Ratio PM Production Rate |
|---|---|
| pUC18-aspC-pno | 1.0 |
| pUC18-aspC-pno-kat | 1.6 |

As set forth in Table 5, it is revealed that in a case in which the recombinant microorganism according to the disclosure further includes the gene encoding a hydrogen peroxide-degrading enzyme, the production efficiency of pyridoxamine or a salt thereof under a condition in which oxygen supply is limited is further improved.

As described above, according to the method of producing pyridoxamine or a salt thereof according to the disclosure, it is shown that pyridoxamine or a salt thereof can be inexpensively produced from pyridoxine or a salt thereof with high production efficiency.

Test 6 (Reference): Examination of Pyridoxine Concentration of Pno

DH5α transformed with the plasmid (pUC18-pno) produced in Comparative Example 1 was inoculated into 2 mL of LB medium including 100 μg/mL of ampicillin in a 500 mL test tube, and cultured with shaking at 37° C. for 24 hours. The culture solution was centrifuged at 13,000 rpm for 3 minutes, and bacterial cells obtained as a precipitate were suspended in 1,000 μL of water to prepare a bacterial cell suspension.

Pyridoxine hydrochloride was dissolved in water to prepare a substrate solution including pyridoxine hydrochloride (500 mM). The substrate solution was adjusted to pH 8.0 with sodium hydroxide. 1,000 μL of reaction liquid was prepared in a 2.0 mL tube by nixing a predetermined amount of substrate solution, 100 μL of Tris-HCl (1 M), 100 μL of bacterial cell suspension, and water so that the concentration of pyridoxine hydrochloride in each reaction liquid was shown in Table 6. The reaction was allowed to proceed with shaking at 1,000 rpm and 37° C. for 24 hours while the tube was capped. A part of the reaction liquid was collected, centrifuged, and the produced pyridoxal hydrochloride was quantified by measuring the absorbance at 415 nm. The amount of PL production at each pyridoxine hydrochloride concentration set forth in Table 6 is expressed in a relative value (ratio of PL production) to the amount of pyridoxal hydrochloride at a pyridoxine hydrochloride concentration of 0.8 mM.

TABLE 6

| Pyridoxine hydrochloride (mM) | Ratio of PL Production |
|---|---|
| 0.8 | 1.00 |
| 4.2 | 1.53 |
| 8.4 | 1.50 |
| 42.1 | 0.98 |
| 84.2 | 0.75 |
| 168.3 | 0.61 |

As set forth in Table 6, in a case in which the concentration of pyridoxine hydrochloride in the reaction liquid was increased to above a certain value, the PL production ratio was rather decreased. In other words, inhibition of the enzymatic activity of pyridoxine oxidase was observed.

Test 7: Production of Pyridoxamine Using Pyridoxine as Raw Material (Raw Material Added all at Once)

DH5α transformed with the plasmid (pUC18-ppat-pno) produced in Example 1 was inoculated into 400 mL of LB medium including 100 μg/mL of ampicillin in a 2,000 mL baffled Erlenmeyer flask and cultured with shaking at 30° C. for 24 hours. The culture solution was centrifuged at 8,000 rpm for 20 minutes, and bacterial cells obtained as a precipitate were suspended in 10 mL of water to prepare a bacterial cell suspension.

Pyridoxine hydrochloride was dissolved in water and adjusted to pH 8.0 with sodium hydroxide to prepare a substrate solution (1) including pyridoxine hydrochloride (500 mM). L-alanine was dissolved in water and adjusted to pH 8.0 with sodium hydroxide to prepare a substrate solution (2) including L-alanine (1,000 mM). Into a 100 mL baffled Erlenmeyer flask, 4.0 mL of cell suspension, 4.0 mL of the substrate solution (1), and 2.0 mL of the substrate solution (2) were added, which was allowed to react with shaking at 200 rpm for 24 hours at 37° C. A part of the reaction liquid was collected, and the concentration of pyridoxamine dihydrochloride was analyzed under the analysis conditions described above. The results are shown in FIG. 1. In FIGS. 1 to 3, PN represents the concentration of pyridoxine hydrochloride, PL represents the concentration of pyridoxal hydrochloride, and PM represents the concentration of pyridoxamine dihydrochloride. Further, hr represents a unit of time “hour”.

As shown in FIG. 1, production of pyridoxamine dihydrochloride proceeded even when high concentrations of pyridoxine hydrochloride and L-alanine were all added from the beginning.

Test 8: Production of Pyridoxamine Using Pyridoxine as Raw Material (Pyridoxine Hydrochloride and L-Alanine Added in Several Batches)

DH5α transformed with the plasmid (pUC18-ppat-pno) produced in Example 1 was inoculated into 400 mL of LB medium including 100 μg/mL of ampicillin in a 2,000 mL baffled Erlenmeyer flask and cultured with shaking at 30° C. for 24 hours. The culture solution was centrifuged at 8,000 rpm for 20 minutes, and bacterial cells obtained as a precipitate were suspended in 10 mL of water to prepare a bacterial cell suspension.

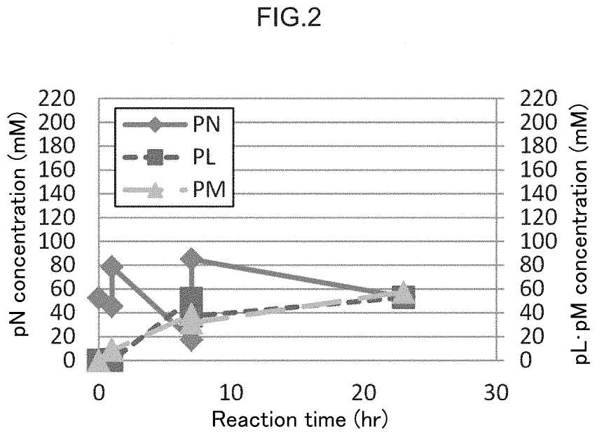

Pyridoxine hydrochloride was dissolved in water and adjusted to pH 8.0 with sodium hydroxide to prepare a substrate solution (1) including pyridoxine hydrochloride (500 mM). L-alanine was dissolved in water and adjusted to pH 8.0 with sodium hydroxide to prepare a substrate solution (2) including L-alanine (1,000 mM). Into a 100 mL baffled Erlenmeyer flask, 4.0 mL of cell suspension, 0.5 mL of the substrate solution (1), and 0.25 mL of the substrate solution (2) were added, which was allowed to react with shaking at 200 rpm at 37° C. One hour after the initiation of the reaction, 0.5 mL of the substrate solution (1) and 0.25 mL of the substrate solution were further added. Seven hours after the initiation of the reaction, 0.5 mL of the substrate solution (1) and 0.25 mL of the substrate solution (2) were further added. The reaction was continued until 24 hours after the initiation of the reaction. A part of the reaction liquid was collected, and the concentration of pyridoxamine dihydrochloride was analyzed under the analysis conditions described above. The results are shown in FIG. 2. In this test, equimolar amounts of pyridoxine hydrochloride and L-alanine were added each time.

As shown in FIG. 2, the production efficiency of pyridoxamine dihydrochloride was further improved when a relatively large amount of pyridoxine hydrochloride was added in several batches rather than adding the whole amount from the beginning. It is assumed that the concentration of pyridoxine can be maintained relatively low due to the addition in several batches, and the pyridoxine oxidase activity can be more easily exhibited.

Test 9: Production of Pyridoxamine Using Pyridoxine as Raw Material (Pyridoxine Hydrochloride and L-Alanine Added in Several Batched and L-Alanine Added at Once)

DH5α transformed with the plasmid (pUC18-ppat-pno) produced in Example 1 was inoculated into 400 mL of LB medium including 100 μg/mL of ampicillin in a 2,000 mL baffled Erlenmeyer flask and cultured with shaking at 30° C. for 24 hours. The culture solution was centrifuged at 8,000 rpm for 20 minutes, and bacterial cells obtained as a precipitate was suspended in 10 mL of water to prepare a bacterial cell suspension.

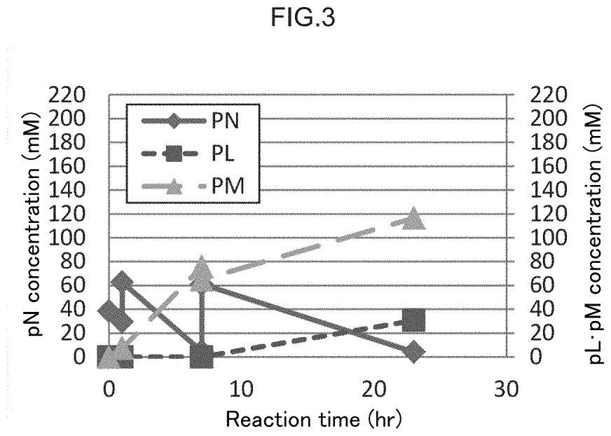

Pyridoxine hydrochloride was dissolved in water and adjusted to pH 8.0 with sodium hydroxide to prepare a substrate solution (1) including pyridoxine hydrochloride (500 mM). L-alanine was dissolved in water and adjusted to pH 8.0 with sodium hydroxide to prepare a substrate solution (2) including L-alanine (1,000 mM). Into a 100 mL baffled Erlenmeyer flask, 4.0 mL of cell suspension, 0.5 mL of the substrate solution (1), and 2.0 mL of the substrate solution (2) were added, which was allowed to react with shaking at 200 rpm at 37° C. One hour after the initiation of the reaction, 0.5 mL of the substrate solution (1) was further added. Seven hours after the initiation of the reaction, 0.5 mL of the substrate solution (1) was further added. The reaction was continued until 24 hours after the initiation of the reaction. A part of the reaction liquid was collected, and the concentration of pyridoxamine dihydrochloride was analyzed under the analysis conditions described above. The results are shown in FIG. 3. In this test, the molar concentration of L-alanine was always maintained higher than the molar concentration of pyridoxine hydrochloride.

As can be seen from the comparison of FIG. 2 with FIG. 3, in a case in which the concentration of L-alanine was maintained higher than the concentration of pyridoxine hydrochloride, the production efficiency of pyridoxamine dihydrochloride was further improved even with a relatively large the amount of pyridoxine hydrochloride. It is assumed that due to the presence of L-alanine at a higher concentration than pyridoxine hydrochloride, the reaction equilibrium was more advantageously shifted in favor of the pyridoxamine generation.

### Reference Example 1: Production of ppat Expression Strain

A synthetic DNA having the nucleotide sequence of SEQ ID NO:10 was obtained by custom synthesis of the nucleotide sequence of a pyridoxamine-pyruvate aminotransferase gene derived from Mesorhizobium loti MAFF303099, codon-optimized for E. coli, from GenScript. A DNA fragment including a target gene was amplified by PCR with an oligonucleotide having the nucleotide sequence of SEQ ID NO:51 and an oligonucleotide having the nucleotide sequence of SEQ ID NO:52 as primers using the synthetic DNA as a template. The amplified DNA fragment was treated with EcoRI and BamHI, and the obtained DNA fragment and a treated product of PUC18 (manufactured by Takara Bio Inc.) with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:10 according to a usual method of determining a nucleotide sequence. The obtained plasmid was named pUC18-ppat. Here, ppat is the abbreviated name of a pyridoxamine-pyruvate aminotransferase gene.

### Reference Example 2: Production of ppat-plr Expression Stain

A synthetic DNA having the nucleotide sequence of SEQ ID NO:53 was obtained by custom synthesis of the nucleotide sequence of a pyridoxal reductase gene derived from Saccharomyces cerevisiae, codon-optimized for E. coli, from GenScript. A DNA fragment including a target gene was amplified by PCR with an oligonucleotide having the nucleotide sequence of SEQ ID NO:54 and an oligonucleotide having the nucleotide sequence of SEQ ID NO:55 as primers using the synthetic DNA as a template. The amplified DNA fragment was treated with Sail and HindIII, and the obtained DNA fragment and a treated product obtained by treating the pUC18-ppat produced in Reference Example 1 with SalI and HindIII were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:53 according to a usual method of determining a nucleotide sequence. The obtained plasmid was named pUC18-ppat-plr. Here, plr is the abbreviated name of a pyridoxal reductase gene (corresponding to pyridoxine dehydrogenase).

### Reference Example 3: Production of ppat-adh Expression Strain

A synthetic DNA having the nucleotide sequence of SEQ ID NO:56 was obtained by custom synthesis of the nucleotide sequence of an alanine dehydrogenase gene derived from Shewanella sp. AC10, into which a mutation as D198A in an encoded amino acid sequence was introduced, and which was codon-optimized for E. coli, from GenScript. A DNA fragment including a target gene was amplified by PCR with an oligonucleotide having the nucleotide sequence of SEQ ID NO:57 and an oligonucleotide having the nucleotide sequence of SEQ ID NO:58 as primers using the synthetic DNA as a template. The amplified DNA fragment was treated with BamHI and Sail, and the obtained DNA fragment and a treated product obtained by treating the pUC18-ppat produced in Reference Example 1 with BamHI and Sail were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:56 according to a usual method of determining a nucleotide sequence. The obtained plasmid was named pUC18-ppat-adh. Here, adh is the abbreviated name of an alanine dehydrogenase gene.

### Reference Example 4: Production of ppat-adh-plr Expression Strain

A synthetic DNA having the nucleotide sequence of SEQ ID NO:56 was obtained by custom synthesis of the nucleotide sequence of an alanine dehydrogenase gene derived from Shewanella sp. AC10, into which a mutation as D198A in an encoded amino acid sequence was introduced, and which was codon-optimized for E. coli, from GenScript. A DNA fragment including a target gene was amplified by PCR with an oligonucleotide having the nucleotide sequence of SEQ ID NO:57 and an oligonucleotide having the nucleotide sequence of SEQ ID NO:58 as primers using the synthetic DNA as a template. The amplified DNA fragment was treated with BamHI and SalI, and the obtained DNA fragment and a treated product obtained by treating the pUC18-ppat-plr produced in Reference Example 2 with BamHI and SalI were ligated using Ligation High (manufactured by TOYOBO Co., Ltd.). Escherichia coli DH5α (manufactured by TOYOBO Co., Ltd.) was transformed with this ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:56 according to a usual method of determining a nucleotide sequence. The obtained plasmid was named pUC18-ppat-adh-plr.

### Reference Example 5: Production of adh-plr Expression Plasmid

A pyridoxal reductase gene (plr) fragment recovered by treating pUC18-ppat-plr produced in Reference Example 2 with SalI and HindIII, an alanine dehydrogenase gene (adh) fragment recovered by treating pUC-ppat-adh produced in Reference Example 3 with BamHI and SalI, and a treated product of pUC18 with BamHI and HindIII were ligated using Ligation High (TOYOBO Co., Ltd.). Escherichia coli DH5α (manufactured by TOYOBO Co., Ltd.) was transformed with the ligation product. The transformant was cultured on LB agar medium, and a strain with a target plasmid was selected from ampicillin resistant strains. The plasmid was extracted from the transformant obtained in such a manner, and the obtained plasmid was named pUC18-adh-plr.

### Reference Example 6: Production of Msppat-adh-plr Expression Plasmid

A synthetic DNA having the nucleotide sequence of SEQ ID NO:32 was obtained by custom synthesis of a gene in which the nucleotide sequence of a pyridoxamine-pyruvate aminotransferase gene derived from Mesorhizobium sp. YR577 was designed to be codon-optimized for E. coli, and in which EcoRI was introduced into the 5′ end and BamHI was introduced into the 3′, from eurofins. A DNA fragment obtained by treating the synthetic DNA with EcoRI/BamHI, and a DNA fragment obtained by treating pUC18-adh-plr produced in Reference Example 5 with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:32 according to a usual method of determining a nucleotide sequence. The obtained plasmid was named pUC18-Msppat-adh-plr.

### Reference Example 7: Production of Psppat-adh-plr Expression Plasmid

A synthetic DNA having the nucleotide sequence of SEQ ID NO:33 was obtained by custom synthesis of a gene in which the nucleotide sequence of a pyridoxamine-pyruvate aminotransferase gene derived from Pseudaminobacter salicylatoxidan was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Reference Example 5 with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:33 according to a usual method of determining a nucleotide sequence. The obtained plasmid was named pUC18-Psppat-adh-plr.

### Reference Example 8: Production of Blppat-adh-plr Expression Plasmid

A synthetic DNA having the nucleotide sequence of SEQ ID NO:34 was obtained by custom synthesis of a gene in which the nucleotide sequence of a pyridoxamine-pyruvate aminotransferase gene derived from Bauldia litoralis was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Reference Example 5 with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:34 according to a usual method of determining a nucleotide sequence. The obtained plasmid was named pUC18-Blppat-adh-plr.

### Reference Example 9: Production of Ssppat-adh-plr Expression Plasmid

A synthetic DNA having the nucleotide sequence of SEQ ID NO:35 was obtained by custom synthesis of a gene in which the nucleotide sequence of a pyridoxamine-pyruvate aminotransferase gene derived from Skermanella stibiiresistens was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating of pUC18-adh-plr produced in Reference Example 5 with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:35 according to a usual method of determining a nucleotide sequence. The obtained plasmid was named pUC18-Ssppat-adh-plr.

### Reference Example 10: Production of Rsppat-adh-plr Expression Plasmid

A synthetic DNA having the nucleotide sequence of SEQ ID NO:36 was obtained by custom synthesis of a gene in which the nucleotide sequence of a pyridoxamine-pyruvate aminotransferase gene derived from Rhizobium sp. AC44/96 was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Reference Example 5 with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:36 according to a usual method of determining a nucleotide sequence. The obtained plasmid was named pUC18-Rsppat-adh-plr.

### Reference Example 11: Production of Etppat-adh-plr Expression Plasmid

A synthetic DNA having the nucleotide sequence of SEQ ID NO:37 was obtained by custom synthesis of a gene in which the nucleotide sequence of a pyridoxamine-pyruvate aminotransferase gene derived from Erwinia toletana was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Reference Example 5 with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:37 according to a usual method of determining a nucleotide sequence. The obtained plasmid was named pUC18-Etppat-adh-plr.

### Reference Example 12: Production of Hgppat-adh-plr Expression Plasmid

A synthetic DNA having the nucleotide sequence of SEQ ID NO:38 was obtained by custom synthesis of a gene in which the nucleotide sequence of a pyridoxamine-pyruvate aminotransferase gene derived from Herbiconiux ginsengi was designed to be codon-optimized for E. coli, and in which EcoRI was introduced into the 5′ end and BamHI was introduced into the 3′ end, from eurofins. A DNA fragment obtained by treating the synthetic DNA with EcoRI/BamHI, and a DNA fragment obtained by treating pUC18-adh-plr produced in Reference Example 5 with EcoRI and BamHI were ligated using Ligation High (manufactured by TOYOBO Co., Ltd.). Escherichia coli DH5α (manufactured by TOYOBO Co., Ltd.) was transformed with the ligation product. The transformant was cultured on an LB agar medium, and a strain having a target plasmid was selected from ampicillin resistant strains. The plasmid was extracted from the transformant obtained in such a manner.

The nucleotide sequence of the DNA fragment introduced into the plasmid was confirmed to be the nucleotide sequence of SEQ ID NO:38 according to a usual method of determining a nucleotide sequence. The obtained plasmid was named pUC18-Hgppat-adh-plr.

Test 10: Production of Pyridoxamine Using Pyridoxine as Raw Material

DH5α transformed with each of the plasmids produced in Reference Examples 6 to 12 was inoculated into 100 mL of LB medium including 100 μg/mL of ampicillin in a 500 mL baffled Erlenmeyer flask and cultured with shaking at 30° C. for 24 hours. 40 mL of culture solution was taken, and centrifuged at 5,000 rpm for 5 minutes, and bacterial cells obtained as a precipitate were suspended in 0.9 mL of water to prepare a bacterial cell suspension.

Pyridoxine hydrochloride and L-alanine were dissolved in water to prepare a substrate solution including pyridoxine hydrochloride (500 mM) and L-alanine (500 mM). The pH of the substrate solution was adjusted to pH 8.0 with 25% aqueous ammonia. 400 μL of substrate solution, 500 μL of water, and 100 μL of bacterial cell suspension were mixed, and allowed to react at 37° C. for 1 hour. A part of the reaction liquid was collected, and analyzed under the analysis conditions described above. The results are set forth in Table 7. The amount of produced pyridoxamine set forth in Table 7 is expressed in a relative amount, with the molar amount of pyridoxamine dihydrochloride produced by the ppat-adh-plr expression strain produced in Reference Example 4 set to 100.

TABLE 7

| | Amount of Produced Pyridoxamine |
|---|---|
| Introduced plasmid | (Relative Value) |
| pUC18-ppat-adh-plr | 100 |
| pUC18-Msppat-adh-plr | 46 |
| pUC18-Psppat-adh-plr | 113 |
| pUC18-Blppat-adh-plr | 34 |
| pUC18-Ssppat-adh-plr | 77 |
| pUC18-Rsppat-adh-plr | 34 |
| pUC18-Etppat-adh-plr | 109 |
| pUC18-Hgppat-adh-plr | 75 |

As set forth in Table 7, pyridoxamine dihydrochloride was able to be also produced from pyridoxine hydrochloride by introducing a pyridoxamine-pyruvate aminotransferase gene derived from Mesorhizobium sp. YR577, a pyridoxamine-pyruvate aminotransferase gene derived from Pseudaminobacter salicylatoxidans, a pyridoxamine-pyruvate aminotransferase gene derived from Bauldia litoralis, a pyridoxamine-pyruvate aminotransferase gene derived from Skermanella stibiiresistens, a pyridoxamine-pyruvate aminotransferase gene derived from Rhizobium sp. AC44/96, a pyridoxamine-pyruvate aminotransferase gene derived from Erwinia toletana, or a pyridoxamine-pyruvate aminotransferase gene derived from Herbiconiux ginsengi, as a gene encoding a pyridoxamine-pyruvate aminotransferase, instead of the pyridoxamine-pyruvate aminotransferase gene derived from Mesorhizobium loti MAFF303099 used in Reference Example 4. Although these Reference Examples are not examples using pyridoxine oxidase but examples using pyridoxine dehydrogenase, these Reference Examples show that these pyridoxamine-pyruvate aminotransferases can function as the pyridoxamine synthetase in the disclosure.

The disclosure of Japanese Patent Application No. 2017-095572 filed on May 12, 2017, is hereby incorporated by reference in its entirety.

All publications, patent applications, and technical standards mentioned in this specification are herein incorporated by reference to the same extent as if each individual document, patent application, or technical standard was specifically and individually indicated to be incorporated by reference.

1. A recombinant microorganism, comprising:
a gene encoding a pyridoxine oxidase, and a gene encoding a pyridoxamine synthetase having an enzymatic activity of synthesizing pyridoxamine from pyridoxal,
wherein each of the gene encoding the pyridoxine oxidase and the gene encoding the pyridoxamine synthetase is introduced from outside of a bacterial cell, or is endogenous to the bacterial cell and has an enhanced expression.

2. The recombinant microorganism according to claim 1, wherein the pyridoxamine synthetase is a pyridoxamine-pyruvate transaminase, a pyridoxamine-oxaloacetate transaminase, an aspartate transaminase, or a pyridoxamine phosphate transaminase.

3. The recombinant microorganism according to claim 1, wherein the pyridoxine oxidase is represented by enzyme number EC 1.1.3.12.

4. The recombinant microorganism according to claim 1, wherein the gene encoding the pyridoxine oxidase is derived from Microbacterium luteolum.

5. The recombinant microorganism according to claim 1, wherein the gene encoding a pyridoxine oxidase:
(a) has a nucleotide sequence of SEQ ID NO:5;
(b) has a nucleotide sequence that hybridizes with DNA having a nucleotide sequence complementary to the nucleotide sequence of SEQ ID NO:5 under a stringent condition, and that encodes a protein having pyridoxine oxidase activity;
(c) has a nucleotide sequence encoding a protein that has an amino acid sequence of SEQ ID NO:1; or
(d) has a nucleotide sequence encoding a protein that has an amino acid sequence having 80% or more sequence identity with the amino acid sequence of SEQ ID NO:1 and that has pyridoxine oxidase activity.

6. The recombinant microorganism according to claim 1, wherein the pyridoxamine synthetase comprises at least one of the following partial amino acid sequence (c), partial amino acid sequence (d), partial amino acid sequence (e), partial amino acid sequence (f), partial amino acid sequence (g), or partial amino acid sequence (h), and has an enzymatic activity of synthesizing pyridoxamine from pyridoxal:
(c) X₈X₉X₁₀X₁₁X₁₂X₁₃ (SEQ ID NO:39)
wherein X₈ represents L, M, I or V,
X₉ represents H or Q,
X₁₀ represents G, C or A,
X₁₁ represents E or D,
X₁₂ represents P or A, and
X₁₃ represents V, I, L or A;
(d) X₁₄X₁₅TPSGTX₁₆X₁₇ (SEQ ID NO:40)
wherein X₁₄ represents H or S,
X₁₅ represents D or E,
X₁₆ represents I, V, or L, and
X₁₇ represents N or T;
(e) X₁₈DX₁₉VSX₂₀X₂₁ (SEQ ID NO:41)
wherein X₁₈ represents V, I, or A,
X₁₉ represents A, T, or S,
X₂₀ represents S, A, or G, and
X₂₁ represents F, W, or V;
(f) X₂₂X₂₃X₂₄KCX₂₅GX₂₆X₂₇P (SEQ ID NO:42)
wherein X₂₂ represents G or S,
X₂₃ represents P, S, or A,
X₂₄ represents N, G, S, A, or Q,
X₂₅ represents L or M,
X₂₆ represents A, S, C, or G, and
X₂₇ represents P, T, S, or A;
(g) X₂₈X₂₉X₃₀X₃₁SX₃₂GX₃₃X₃₄ (SEQ ID NO:43)
wherein X₂₈ represents G or D,
X₂₉ represents V or I,
X₃₀ represents V, T, A, S, M, I, or L,
X₃₁ represents F, M, L, I, or V,
X₃₂ represents S, G, A, T, I, L, or H,
X₃₃ represents R, M, or Q, and
X₃₄ represents G, R, A, D, H, or K; and
(h) X₃₅X₃₆RX₃₇X₃₈HMGX₃₉X₄₀A (SEQ ID NO:44)
wherein X₃₅ represents L or V,
X₃₆ represents T, I, V, or L,
X₃₇ represents I, V, or L,
X₃₈ represents G or S,
X₃₉ represents P, A, or R, and
X₄₀ represents T, V, or S.

7. The recombinant microorganism according to claim 1, wherein the pyridoxamine synthetase is represented by enzyme number EC 2.6.1.30.

8. The recombinant microorganism according to claim 1, wherein the gene encoding a pyridoxamine synthetase is derived from Mesorhizobium loti.

9. The recombinant microorganism according to claim 1, wherein the gene encoding a pyridoxamine synthetase:
(a) has a nucleotide sequence of any one of SEQ ID NO:6 or SEQ ID NO:25 to SEQ ID NO:31, or
has a region between an 18th nucleotide and a 3′ end in a nucleotide sequence of SEQ ID NO:10, or a region between an 18th nucleotide and a 3′ end in a nucleotide sequence of any one of SEQ ID NO:32 to SEQ ID NO:38;
(b) has a nucleotide sequence that hybridizes with DNA having a nucleotide sequence complementary to a nucleotide sequence of any one of SEQ ID NO:6 or SEQ ID NO:25 to SEQ ID NO:31, or with DNA having a nucleotide sequence complementary to a region between an 18th nucleotide and a 3′ end in a nucleotide sequence of SEQ ID NO:10 or a region between an 18th nucleotide and a 3′ end in a nucleotide sequence of any one of SEQ ID NO:32 to SEQ ID NO:38 under a stringent condition, and that encodes a protein having an enzymatic activity of synthesizing pyridoxamine from pyridoxal;
(c) has a nucleotide sequence encoding a protein that has an amino acid sequence of any one of SEQ ID NO:2 or SEQ ID NO:18 to SEQ ID NO:24; or
(d) has a nucleotide sequence encoding a protein that has an amino acid sequence having 80% or more sequence identity with at least one amino acid sequence selected from the group consisting of SEQ ID NO:2 and SEQ ID NO:18 to SEQ ID NO:24, and that has an enzymatic activity of synthesizing pyridoxamine from pyridoxal.

10. The recombinant microorganism according to claim 1, wherein the pyridoxamine synthetase is represented by enzyme number EC 2.6.1.31 or EC 2.6.1.1.

11. The recombinant microorganism according to claim 1, wherein the gene encoding a pyridoxamine synthetase is derived from Escherichia coli.

12. The recombinant microorganism according to claim 1, wherein the gene encoding the pyridoxamine synthetase:
(a) has a nucleotide sequence of SEQ ID NO:8;
(b) has a nucleotide sequence that hybridizes with DNA having a nucleotide sequence complementary to the nucleotide sequence of SEQ ID NO:8 under a stringent condition, and that encodes a protein having an enzymatic activity of synthesizing pyridoxamine from pyridoxal;
(c) has a nucleotide sequence encoding a protein that has an amino acid sequence of SEQ ID NO:4; or
(d) has a nucleotide sequence encoding a protein that has an amino acid sequence having 80% or more sequence identity with the amino acid sequence of SEQ ID NO:4, and that has an enzymatic activity of synthesizing pyridoxamine from pyridoxal.

13. The recombinant microorganism according to claim 1, further comprising a gene encoding a hydrogen peroxide-degrading enzyme that has an enzymatic activity of generating oxygen from hydrogen peroxide.

14. The recombinant microorganism according to claim 13, wherein the gene encoding the hydrogen peroxide-degrading enzyme is introduced from outside of a bacterial cell, or is endogenous to a bacterial cell and has an enhanced expression.

15. The recombinant microorganism according to claim 13, wherein the hydrogen peroxide-degrading enzyme is represented by enzyme number EC 1.11.1.6.

16. The recombinant microorganism according to claim 13, wherein the gene encoding a hydrogen peroxide-degrading enzyme:
(a) has a nucleotide sequence of SEQ ID NO:7;
(b) hybridizes with DNA having a nucleotide sequence complementary to the nucleotide sequence of SEQ ID NO:7 under a stringent condition, and has an enzymatic activity of generating oxygen from hydrogen peroxide;
(c) has a nucleotide sequence encoding a protein that has an amino acid sequence of SEQ ID NO:3; or
(d) has a nucleotide sequence encoding a protein that has an amino acid sequence having 80% or more sequence identity with the amino acid sequence of SEQ ID NO:3, and that has an enzymatic activity of generating oxygen from hydrogen peroxide.

17. The recombinant microorganism according to claim 1, comprising a recombinant E. coli.

18. A method of producing pyridoxamine or a salt thereof, the method comprising bringing the recombinant microorganism according to claim 1, a culture of the recombinant microorganism, or a treated product of the recombinant microorganism or the culture, into contact with pyridoxine or a salt thereof to produce pyridoxamine or a salt thereof in the presence of oxygen.

19. The production method according to claim 18, wherein the recombinant microorganism, the culture of the recombinant microorganism, or the treated product of the recombinant microorganism or the culture, comprises the pyridoxine oxidase and the pyridoxamine synthetase.

20. The production method according to claim 19, wherein the recombinant microorganism, the culture of the recombinant microorganism, or the treated product of the recombinant microorganism or the culture, further comprises a hydrogen peroxide-degrading enzyme.

21. The production method according to claim 18, wherein the treated product of the recombinant microorganism or the culture is a product treated by treatment comprising one or more selected from the group consisting of heat treatment, cooling treatment, mechanical destruction of a cell, ultrasonic treatment, freeze-thaw treatment, drying treatment, pressurized or reduced pressure treatment, osmotic pressure treatment, cell autolysis, surfactant treatment, enzyme treatment, cell separation treatment, purification treatment, and extraction treatment.

22. The production method according to claim 18, the method comprising either or both of the following (A) and (B):
(A) adding pyridoxine or a salt thereof continuously or in several batches to a solution including the recombinant microorganism, the culture of the recombinant microorganism, or the treated product of the recombinant microorganism or the culture; and
(B) controlling a molar concentration of an amino acid consumed by the pyridoxamine synthetase so as to be 1 or more times a molar concentration of pyridoxine or a salt thereof, in a solution including the recombinant microorganism, the culture of the recombinant microorganism, or the treated product of the recombinant microorganism or the culture.

23. The production method according to claim 22, wherein the amino acid consumed by the pyridoxamine synthetase is L-alanine, D-alanine, L-glutamic acid, or D-glutamic acid.
